(19) United States
(12) Patent Application Publication
(10) Pub. No.: US 2017/0205396 A1
(43) Pub. Date: Jul. 20, 2017

(54) SYSTEMS AND METHODS FOR CULTURING NEPHRON PROGENITOR CELLS

(71) Applicant: Salk Institute for Biological Studies, La Jolla, CA (US)

(72) Inventors: Juan Carlos IZPISUA BELMONTE, La Jolla, CA (US); Zhongwei LI, La Jolla, CA (US); Toshikazu ARAOKA, La Jolla, CA (US); Jun WU, La Jolla, CA (US)

(21) Appl. No.: 15/406,606

(22) Filed: Jan. 13, 2017

Related U.S. Application Data
(60) Provisional application No. 62/279,593, filed on Jan. 15, 2016.

(51) Int. Cl. G01N 33/50; A61K 35/22; C12N 5/071

(52) U.S. Cl. CPC G01N 33/5014; G01N 33/5088; C12N 5/0697; C12N 5/0687; A61K 35/22; G01N 2500/10; C12N 2501/115; C12N 2501/155; C12N 2501/91; C12N 2501/727; C12N 2501/235

(57) ABSTRACT

Provided herein are self-renewable nephron progenitor cell (srNPC) and methods for making and using.

### CROSS-REFERENCE

This application claims the benefit of priority from U.S. Provisional Patent Application No. 62/279,593, filed Jan. 15, 2016, which is herein incorporated by reference in its entirety.

### BACKGROUND

Nephron progenitors, a self-renewing stem cell population in the developing embryo, in some embodiments, give rise to all nephrons, the functional unit of the kidney. In mammals, including humans, in some embodiments, nephron progenitors are evanescent in development and are absent in the adult. Acute or progressive destruction of nephrons due to trauma, disease, or aging eventually lead to end-stage renal disease.

### SUMMARY OF THE INVENTION

Provided herein are nephron progenitor cells and methods of making and using. In some embodiments, there are provided methods of producing a self-renewable nephron progenitor cell (srNPC) comprising: a) purifying a population of Six2+ cells from a mammal; b) culturing the population of Six2+ in a composition comprising NPSR (nephron progenitor self-renewal) media; and c) isolating the floating aggregated cells. In some aspects, the cells are dissociated and passaged every 1 to 14 days. In some aspects, the cells are dissociated and passaged every 4 to 7 days. In some aspects, the population of Six2+ cells is cultured at a density of about 100 to about 20000 cells per well in a 96-well plate, or equivalent density in a smaller or larger plate. In some aspects, the population of Six2+ cells is cultured at a density of about 100 to about 400 cells, about 300 to about 500 cells, about 400 to about 800 cells, about 500 to about 1000 cells, about 1000 to about 2000 cells, about 2000 to about 3000 cells, about 3000 to about 4000, about 4000 to about 5000 cells, about 5000 to about 6000 cells, about 4000 to about 6000 cells, about 6000 to about 7000 cells, about 7000 to about 8000 cells, about 8000 to about 9000 cells, about 9000 to about 10000 cells, about 10000 cells to about 12000 cells, about 12000 to about 14000 cells, about 14000 to about 16000 cells, about 16000 to about 18000 cells, about 18000 to about 20000 cells per well in a 96-well plate, or equivalent density in a smaller or larger plate. In some aspects, the population of Six2+ cells is cultured at a density of about 1000 to about 10000 cells per well in a 96-well plate, or an equivalent density in a smaller or larger plate. In some aspects, the population of Six2+ cells is cultured at a density of about 2500 to about 7500 cells per well in a 96-well plate, or an equivalent density in a smaller or larger plate. In some aspects, the population of Six2+ cells is cultured at a density of about 5000 cells per well in a 96-well plate, or equivalent density in a smaller or larger plate. In some aspects, the population of Six2+ cells is cultured at a density of 5000 cells per well in a 96-well plate, or equivalent density in a smaller or larger plate. In some aspects, the 96-well plate is a U bottom low-attachment plate. In some aspects, the NPSR media comprises a BMP, a FGF, a heparan sulfate, a kinase inhibitor, a cytokine, or combinations thereof. In some aspects, the NPSR media comprises BMP7, FGF2, Heparin, Y27632, LIF, CHIR99021, or combinations thereof.

Also provided herein, in some embodiments are methods of producing a self-renewable nephron progenitor cell (srNPC) comprising: a) culturing a population of cells comprising kidney progenitors in a composition comprising nephron progenitor self-renewal (NPSR) media and b) isolating the floating aggregated cells. In some aspects, the cells are dissociated and passaged every 1 to 14 days. In some aspects, the cells are dissociated and passaged every 4 to 7 days. In some aspects, the population of cells is cultured at a density of about 100 to about 20000 cells per well of a 12-well plate, or an equivalent density in a smaller or larger plate. In some aspects, the population of cells is cultured at a density of about 5000-about 10000 cells per well of a 12-well plate, or an equivalent density in a smaller or larger plate. In some aspects, the population of cells is cultured at a density of about 100 to about 400 cells, about 300 to about 500 cells, about 400 to about 800 cells, about 500 to about 1000 cells, about 1000 to about 2000 cells, about 2000 to about 3000 cells, about 3000 to about 4000, about 4000 to about 5000 cells, about 5000 to about 6000 cells, about 4000 to about 6000 cells, about 6000 to about 7000 cells, about 7000 to about 8000 cells, about 8000 to about 9000 cells, about 9000 to about 10000 cells, about 10000 cells to about 12000 cells, about 12000 to about 14000 cells, about 14000 to about 16000 cells, about 16000 to about 18000 cells, about 18000 to about 20000 cells per well in a 12-well plate, or equivalent density in a smaller or larger plate. In some aspects, the 12-well plate is coated. In some aspects, the 12-well plate is laminin coated. In some aspects, the NPSR media comprises a BMP, a FGF, a heparan sulfate, a kinase inhibitor, a cytokine, or combinations thereof. In some aspects, the NPSR media comprises BMP7, FGF2, Heparin, Y27632, LIF, CHIR99021, or combinations thereof.

Also provided herein, in some embodiments, are methods of producing a self-renewable nephron progenitor cell (srNPC) comprising: a) purifying a population of EpCAM−/NGFR+ cells; b) culturing the population of EpCAM−/NGFR+ cells in a composition comprising nephron progenitor self-renewal (NPSR) media; and c) isolating the floating aggregated cells. In some aspects, the cells are passaged every 1 to 14 days. In some aspects, the cells are passaged every 4 to 7 days. In some aspects, the population of EpCAM−/NGFR+ cells are cultured at a density of about 100 to about 50000 cells per well of a 96-well plate, or an equivalent density in a smaller or larger plate. In some aspects, the population of EpCAM−/NGFR+ cells is cultured at a density of about 100 to about 400 cells, about 300 to about 500 cells, about 400 to about 800 cells, about 500 to about 1000 cells, about 1000 to about 2000 cells, about 2000 to about 3000 cells, about 3000 to about 4000, about 4000 to about 5000 cells, about 5000 to about 6000 cells, about 4000 to about 6000 cells, about 6000 to about 7000 cells, about 7000 to about 8000 cells, about 8000 to about 9000 cells, about 9000 to about 10000 cells, about 10000 cells to about 12000 cells, about 12000 to about 14000 cells, about 14000 to about 16000 cells, about 16000 to about 18000 cells, about 18000 to about 20000 cells, about 20000 to about 24000 cells, about 24000 to about 28000 cells, about 28000 to about 32000 cells, about 30000 to about 35000 cells, about 35000 to about 40000 cells, about 40000 to about 45000 cells, about 45000 to about 50000 cells per well in a 96-well plate, or equivalent density in a smaller or larger plate. In some aspects, the population of EpCAM−/NGFR+ cells is cultured at a density of about 5000 to about 20000 cells per well in a 96-well plate, or an equivalent density in a smaller or larger plate. In some aspects, the population of EpCAM−/NGFR+ cells is cultured at a density of about 7500 to about 12000 cells per well in a 96-well plate, or an equivalent density in a smaller or larger plate. In some aspects the population of EpCAM− NGFR+ cells is cultured at a density of about 10000 cells per well in a 96-well plate, or equivalent density in a smaller or larger plate. In some aspects, the population of EpCAM−/NGFR+ cells are cultured at a density of 10000 cells per well of a 96-well plate, or an equivalent density in a smaller or larger plate. In some aspects, the 96-well plate is a U bottom low attachment plate. In some aspects, the NPSR media comprises a BMP, a FGF, a heparan sulfate, a kinase inhibitor, a cytokine, or combinations thereof. In some aspects, the NPSR media comprises BMP7, FGF2, Heparin, Y27632, LIF, CHIR99021, or combinations thereof. In some aspects, the population of cells is derived from an embryonic kidney, a fetal kidney, an adult kidney, or menstrual fluid. In some aspects, the cells are derived from a mammal. In some aspects, the mammal is mouse, rat, pig, dog, cat, rabbit, cow, horse, camel, goat, or human. In some aspects, the population of cells is Six2+.

In some embodiments, there are provided methods of producing a fate-specified nephron progenitor cell comprising contacting a self-renewable nephron progenitor cell (srNPC) made by a method provided herein with a spinal cord tissue, a kinase inhibitor, a growth factor, or a combination thereof. In some aspects, the srNPC is contacted with a spinal cord tissue, CHIR99021, FGF2, or combinations thereof.

In some embodiments, there is provided, a kit comprising nephron progenitor self-renewal (NPSR) media and instructions for making a srNPC or fate-specified nephron progenitor cells (FS-NPC) according to the methods provided herein.

Also provided herein are methods of treating a subject with a kidney disease comprising administering an effective amount of the self-renewable nephron progenitor cells (srNPC) or fate-specified nephron progenitor cells (FS-NPC) made by the method provided herein. In some aspects, the kidney disease is acute or chronic. In some aspects, the kidney disease is selected from one or more of analgesic nephropathy, diabetic nephropathy, IgA nephropathy, contrast induced nephropathy, lithium induced nephropathy, xanthine oxidase deficiency, polycystic kidney disease, onconephrology, renal insufficiency, renal failure, or uremia. In some aspects, the subject is a mammal. In some aspects, the subject is a human.

In some embodiments, there are provided self-renewable nephron progenitor cells (srNPC) or fate-specified nephron progenitor cells (FS-NPC) made by a method provided herein for use in treating kidney disease.

Also provided herein, in some embodiments, are kits for treating kidney disease in a subject comprising the self-renewable nephron progenitor cells (srNPC) or fate-specified nephron progenitor cells (FS-NPC) made by a method described herein and instructions for use.

Provided herein, in some embodiments, are methods of screening a drug for kidney toxicity comprising: a) creating a nephron organoid created by contacting a self-renewable nephron progenitor cells (srNPC) made by a method provided herein with a composition comprising CHIR99021 and FGF2; b) contacting the nephron organoid with the drug; and c) detecting signs of toxicity. In some aspects, the signs of toxicity comprise changes in glomerulus, proximal tubule, distal tubule, loop of Henle morphology, or cellular apoptosis.

Also provided herein are isolated self-renewable nephron progenitor cells (srNPC), wherein the srNPC is capable of differentiating into nephron or kidney structures in vitro or in vivo and wherein the srNPC is SIX2+. In some aspects, the srNPC is derived from an embryonic or fetal kidney cell. In some aspects, the srNPC is derived from an adult kidney cell. In some aspects, the srNPC is derived from a menstrual fluid. In some aspects, the srNPC is derived from a mammal. In some aspects, the mammal is a mouse, rat, pig, dog, cat, rabbit, cow, horse, camel, goat, or human.

Also provided herein are isolated fate-specified nephron progenitor cells (FS-NPC), wherein the FS-NPC is capable of differentiating into nephron or kidney structures in vitro or in vivo. In some aspects, the FS-NPC is SIX2+, LIM1+, PAX8+, or a combination thereof. In some aspects, the FS-NPC is derived from an embryonic or fetal kidney cell. In some aspects, the FS-NPC is derived from an adult kidney cell. In some aspects, the FS-NPC is derived from a menstrual fluid. In some aspects, the FS-NPC is derived from a mammal. In some aspects, the mammal is a mouse, rat, pig, dog, cat, rabbit, cow, horse, camel, goat, or human.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1A shows a scheme of the experimental protocol for NPSR-based 3D aggregate culture of primary NPCs (srNPCs).

FIG. 1B shows representative time-lapse bright field images showing the morphology and size of srNPC aggregates within one passage cycle. Scale bar, 200 μm.

FIG. 1C shows a growth curve of srNPCs within one passage cycle.

FIG. 1D shows doubling times of srNPCs calculated from indicated passages.

FIG. 1E shows percentage of SIX2+, SALL1+ or CITED+. Data presented as mean±SD.

FIG. 1F shows Western blotting analysis for expression of NPC markers SIX2, PAX2, WT1, mesenchymal markers VIMENTIN and TWIST, and epithelial marker E-cadherin (CDH1) in srNPCs derived from E13.5 embryonic kidney (P60). Mouse embryonic stem cells (mESCs) were used as a control. ACTIN was used as a loading control.

FIG. 1G shows schematic representation of the different developmental time points (E11.5, E13.5, E16.5, P1) chosen for srNPCs derivation. Lower panel summarizes the srNPC derivation efficiency from the different development stages.

FIG. 2A shows a bright field image of the spinal cord induction assay. E13.5-derived srNPCs were co-cultured with E12.5 dorsal spinal cord for 7 days. The arrow indicates the differentiated structures from srNPCs. Dashed line indicates the boundary between spinal cord and differentiated srNPCs. SP, spinal cord. Scale bar, 1 mm.

FIG. 2B shows a higher magnification a bright field image of the spinal cord induction assay as in FIG. 2A. Scale bar, 1 mm.

FIG. 2C shows a schema of the complementary reaggregation assay. E11.5 mouse embryonic kidneys from Six2^{GCE} mouse strain were dissociated into single cells followed by FACS sorting. Six2-GFP− populations were collected and mixed with mCherry-labeled srNPCs. Formed reaggregates were transferred to air-liquid interface for further culture.

FIG. 2D shows bright field images showing morphologies of indicated aggregates cultured in air-liquid interface at different time points. Scale bar, 200 μm.

FIG. 3A shows a schematic representation of the differentiation from srNPCs to nephron organoids through fate-specified NPCs (FS-NPCs). Representative bright field images are shown for each stage.

FIG. 3B shows representative bright field image showing the morphology of part of the nephron organoid differentiated from srNPCs. Scale bar, 200 μm.

FIG. 3C shows qRT-PCR analyses of pertubular aggregates (PTA) and renal vesicles (RV) markers Lhx1 and Pax8, and NPC markers Six2, Wt1, and Osr1 in mESCs (control) (left bar), srNPCs (center bar) and FS-NPCs (right bar). Data are presented as mean±SD.

FIG. 3D shows a schematic representation of the experimental protocol for the culture-dependent purification (CDP) method.

FIG. 3E shows qRT-PCR analysis of Cd2ap gene expression in three srNPCs lines derived from tetO transgenic mice (#1 and #3, homozygotes; #2, wild type). Data are presented as mean±SD.

FIG. 4A shows a cartoon showing the experimental procedure for injecting mCherry-labeled mouse FS-NPCs into the kidney of neonatal mice (P1) and analyzing FS-NPCs contribution to P8 kidney.

FIG. 4B shows a cartoon showing the experimental procedure for grafting mCherry-labeled mouse srNPCs to the coelomic cavity of the HH18 stage chick embryo followed by analysis of cell contribution 7 days after grafting.

FIG. 5A shows creatinine levels in urine and cyst fluid. Urine samples were obtained from three wild type mice (n=3) and cyst fluids were collected from three mice receiving srNPC transplantation (n=3). Data are presented as mean±SD.

FIG. 5B shows mouse survival using Kapan-Meier survival curves. The immunodeficient NSG mice received a subcutaneous injection of cisplatin (20 mg/Kg) 24 hr prior to subcapsule transplantation either with PBS (n=5) or mouse FS-NPCs (n=6).

FIG. 5C shows blood urea nitrogen (BUN) and serum Creatinine (S-Cre) levels in cisplatin induced AKI mice receiving renal subcapsular transplantation of mouse FS-NPCs (n=5) or PBS (n=5). Control mice (n=7) received renal subcapsule transplantation of PBS without cisplatin treatment. Data are presented as mean±SD.

FIG. 5D shows counting of pathological features of histological sections from host kidney transplanted with FS-NPCs (n=3) or PBS (n=4) 4 days after cisplatin administration. Data are presented as mean±SD.

FIG. 6A shows a schematic representation of the purification and derivation of human srNPCs from human fetal kidney.

FIG. 6B shows a growth curve of human srNPCs.

FIG. 6C shows percentage of SIX2+, SALL1+, or WT1+ cells from immunofluorescence analyses of human srNPCs after two-month culture for NPC markers SIX2, SALL1, and WT1. Data presented as mean±SD.

FIG. 6D shows a schematic representation of complementary reaggregation assay for human srNPCs. The procedure is similar to the complementary reaggregation assay described in FIG. 2C, except that a mixture of human srNPCs (80%) and mouse srNPCs (20%) were used.

FIG. 6E shows blood urea nitrogen (BUN) and serum Creatinine (S-Cre) levels in cisplatin-induced acute kidney injury mice that received the renal subcapsular transplantation of either human FS-NPCs (n=3) or saline (n=4). Control mice (n=7) received renal subcapsule transplantation of PBS without cisplatin injection. Data are presented as mean±SD.

FIG. 6F shows quantification of the incidence of several pathological features of histological sections associated with cisplatin-induced nephrotoxicity of host kidney transplanted with human FS-NPCs or saline (control) 4 days post-cisplatin administration. Data are presented as mean±SD.

### DETAILED DESCRIPTION OF THE INVENTION

Disclosed herein are self-renewable nephron progenitor cells (srNPCs) and fate-specified nephron progenitor cells (FS-NPCs) and methods of making and using in therapeutic and research applications.

### Self-Renewable Nephron Progenitor Cells and Fate-Specified Nephron Progenitor Cells

Disclosed herein are self-renewable nephron progenitor cells (srNPCs) and fate-specified nephron progenitor cells (FS-NPCs) that are capable of generating nephron or kidney structures in vitro or in vivo. In some cases, the srNPCs or FS-NPCs are capable of repairing a nephron or kidney structure in vitro or in vivo. In some cases, the srNPCs or FS-NPCs are capable replacing defective nephron or kidney structures in vitro. In some cases, the srNPCs or FS-NPCs are capable of generating nephron or kidney structures in vitro that can be transplanted into a subject in need thereof.

In some cases, srNPCs and FS-NPCs are characterized by markers expressed on the surface of the srNPCs or FS-NPCs. Markers or cell surface markers, in some cases, are biomolecules, such as proteins, carbohydrates, lipids, nucleic acids, or combinations thereof, that are detected by cell surface staining or intracellular staining, such as immunofluorescence, fluorescence-activated cell sorting (FACS), reporter gene assays, or other methods known by those of skill in the art. In some cases, the markers or cell surface markers are detected by antibody staining. In some cases, the markers or cell surface markers are tested by Western blot, ELISA, Northern blot, RT-PCR, quantitative RT-PCR, RNA-seq, or other gene expression quantification method known in the art. In some cases, the markers or cell surface markers are proteins known to be exclusively expressed intracellularly or on the surface of srNPCs or FS-NPCs. In some cases, the markers or cell surface markers comprise one or more of SIX2, ITGA8, PDGFRA, CDH4, NGFR, RET, CDH1, CLDN3, KRT8, PDGFRB, EPCAM, OR VCAM1.

In some cases, srNPCs and FS-NPCs are characterized by global gene expression patterns in the cells. In some cases, global gene expression patterns are determined by RNA-seq, microarray, quantitative RT-PCR, Northern blot, or other gene expression profiling methods known in the art. In some cases, a global gene expression pattern means that some genes are expressed at high levels while other genes are expressed at low levels or not at all. In some cases, Six2, Cited1, Eya1, Wt1, Sal1, Osr1, Gdnf1, Hoxa11, Fgf9, Ret, Gfra1, Gata3, Foxd1, Itga8, or Pdgfra are differentially expressed in srNPCs and FS-NPCs when compared to other cells. In some cases, Six2, Cited1, Eya1, Wt1, Sal1, Osr1, Gdnf1, Hoxa11, Fgf9, or Itga8 are expressed at high levels. In some cases, Ret, Gfra1, Gata3, Foxd1, or Pdgfra are expressed at low levels or not at all. In some cases, a differentially expressed gene is selected from one or more of the genes described in Table 1.

In some cases, the srNPCs and FS-NPCs are cultured in a defined media or nephron progenitor self-renewal (NPSR) media that enables unlimited growth, expansion, or passaging of the cells. In some cases, the defined media allows growth and expansion of the cells for 50 to 10000 passages or more. In some cases, the defined media allows for growth and expansion of the cells for 50-100, 100-200, 200-400, 400-800, 800-1000, 1000-2000, 2000-5000, 5000-10000, or more passages, or any intermediate number of passages thereof.

In some cases, the defined media for culture of the srNPCs and FS-NPCs nephron progenitor self-renewal (NPSR) media comprises a selection of growth factors, cytokines, inhibitors, proteoglycans, or combinations thereof. In some cases, the growth factors comprise a bone morphogenic protein (BMP), a fibroblast growth factor (FGF), a WNT pathway growth factor, or combinations thereof. In some cases, the bone morphogenic protein (BMP) comprises BMP7, BMP2, GDF-5, GDF-8, GDF-3, GDF-11, GMP6, BMP-10, or combinations thereof. In some cases, the bone morphogenic protein (BMP) comprises BMP7. In some cases, the bone morphogenic protein or BMP7 is in the defined media at a concentration that is most effective for the culture of srNPCs and FS-NPCs. In some cases, the concentration of bone morphogenic protein or BMP7 is between 5 ng/ml and 200 ng/ml, for example 5 ng/ml, 6 ng/ml, 7 ng/ml, 8 ng/ml, 9 ng/ml, 10 ng/ml, 11 ng/ml, 12 ng/ml, 13 ng/ml, 14 ng/ml, 15 ng/ml, 20 ng/ml, 25 ng/ml, 50 ng/ml, 75 ng/ml, 100 ng/ml, 125 ng/ml, 150 ng/ml, 175 ng/ml, and 200 ng/ml. In some cases, the concentration of BMP7 used in the defined media is 10 ng/ml. In some cases, the fibroblast growth factor (FGF) comprises FGF2, FGF9, FGF20, FGF1, FGF4, FGF19, FGF21, FGF23, or combinations thereof. In some cases, the fibroblast growth factor (FGF) comprises FGF2. In some cases, the fibroblast growth factor or FGF2 is in the defined media at a concentration that is the most effective for the culture of srNPCs and FS-NPCs. In some cases, the concentration of fibroblast growth factor or FGF2 is between 5 ng/ml and 500 ng/ml, for example 5 ng/ml, 10 ng/ml, 15 ng/ml, 16 ng/ml, 17 ng/ml, 18 ng/ml, 19 ng/ml, 20 ng/ml, 21 ng/ml, 22 ng/ml, 23 ng/ml, 24 ng/ml, 25 ng/ml, 50 ng/ml, 75 ng/ml, 100 ng/ml, 125 ng/ml, 150 ng/ml, 175 ng/ml, and 200 ng/ml, 250 ng/ml, 300 ng/ml, 350 ng/ml, 400 ng/ml, 450 ng/ml, and 500 ng/ml. In some cases, the concentration of FGF2 in the defined media is 20 ng/ml. In some cases, the WNT pathway growth factor comprises Wnt3, Wnt-3a, Wnt7b, Wnt-5a, or combinations thereof. In some cases, the WNT pathway growth factor is in the defined media at a concentration that is the most effective for the culture of srNPCs and FS-NPCs. In some cases, the concentration of WNT pathway growth factor is between 5 ng/ml and 500 ng/ml, for example 5 ng/ml, 10 ng/ml, 15 ng/ml, 20 ng/ml, 25 ng/ml, 50 ng/ml, 75 ng/ml, 100 ng/ml, 125 ng/ml, 150 ng/ml, 175 ng/ml, and 200 ng/ml, 250 ng/ml, 300 ng/ml, 350 ng/ml, 400 ng/ml, 450 ng/ml, and 500 ng/ml. In some cases, the cytokine comprises LIF, IL-6, IL-11, or combinations thereof. In some cases, the cytokine comprises LIF. In some cases, the cytokine or LIF is in the defined media at a concentration that is the most effective for the culture of srNPCs and FS-NPCs. In some cases, the concentration of cytokine or LIF is between 1 ng/ml and 50 ng/ml, for example 1 ng/ml, 2 ng/ml, 3 ng/ml, 4 ng/ml, 5 ng/ml, 10 ng/ml, 15 ng/ml, 20 ng/ml, 25 ng/ml, 30 ng/ml, 40 ng/ml, and 50 ng/ml. In some cases, the concentration of LIF in the defined media is 2 ng/ml. In some cases, the inhibitor comprises a kinase inhibitor such as an inhibitor of p160ROCK, PKC, PKA, MLCK, or combinations thereof. In some cases, the inhibitor comprises Y-27632. In some cases, the p160ROCK inhibitor or Y-27632 is in the defined media at a concentration that is most effective for the culture of srNPCs and FS-NPCs. In some cases, the concentration of p160ROCK inhibitor or Y27632 is between 1 μM and 20 μM, for example 1 μM, 1.25 μM, 1.5 μM, 1.75 μM, 2 μM, 3 μM, 4 μM, 5 μM, 6 μM, 7 μM, 8, μM, 9 μM, 10 μM, 11 μM, 12 μM, 13 μM, 14 μM, 15 μM, 16 μM, 17 μM, 18 μM, 19 μM, and 20 μM. In some cases, the concentration of Y27632 in the defined media is 1 μM. In some cases, the inhibitor comprises a kinase inhibitor such as a glycogen synthase kinase (GSK) inhibitor. In some cases, the GSK inhibitor is a GSK-3, GSK-3α, or GSK-3β inhibitor. In some cases, the GSK-3 inhibitor is CHIR99021. In some cases, the GSK inhibitor or CHIR99021 is in the defined media at a concentration that is most effective for the culture of srNPCs and FS-NPCs. In some cases, the concentration of GSK inhibitor or CHIR99021 is between 0.5 μM and 20 μM, for example 0.5 μM, 0.6 μM, 0.7 μM, 0.8 μM, 0.9 μM, 1 μM, 1.25 μM, 1.5 μM, 1.75 μM, 2 μM, 3 μM, 4 μM, 5 μM, 6 μM, 7 μM, 8, μM, 9 μM, 10 μM, 11 μM, 12 μM, 13 μM, 14 μM, 15 μM, 16 μM, 17 μM, 18 μM, 19 μM, and 20 μM. In some cases, the concentration of CHIR99021 in the defined media is 0.5 μM. In some cases, the proteoglycan is a heparan sulfate. In some cases, the heparan sulfate is heparan sulfate proteoglycan 2, heparin, or combinations thereof. In some cases, the heparan sulfate is heparin. In some cases, the proteoglycan or heparin is in the defined media at a concentration that is most effective for the culture of srNPCs and FS-NPCs. In some cases, the concentration of proteoglycan or heparin is between 100 ng/ml and 10 μg/ml, for example 100 ng/ml, 125 ng/ml 150 ng/ml, 175 ng/ml, 200 ng/ml, 250 ng/ml, 300 ng/ml, 350 ng/ml, 400 ng/ml, 450 ng/ml, 500 ng/ml, 600 ng/ml, 700 ng/ml, 800 ng/ml, 900 ng/ml, 1 μg/ml, 2 μg/ml, 3 μg/ml, 4 μg/ml, 5 μg/ml, 6 μg/ml, 7 μg/ml, 8 μg/ml, 9 μg/ml, and 10 μg/ml. In some cases, the concentration of heparin in the defined media is 100 ng/ml. In some cases, the defined media for culture of the srNPCs and FS-NPCs comprises a composition comprising the defined media and at least one cell, for example a srNPC or a FS-NPC.

In some cases, the srNPCs and FS-NPCs are derived from other cells. In some cases, the other cells are derived from an embryonic or fetal kidney. In some cases, the other cells are derived from neonatal kidney. In some cases, the other cells are derived from adult tissue. In some cases, the other cells are derived from urogenital tissue. In some cases, the other cells are derived from menstrual fluid.

Also disclosed herein are compositions comprising isolated srNPCs or FS-NPCs and a buffer or culture media. In some embodiments, the buffer is a physiologically acceptable buffer. In some embodiments, the buffer is a carrier acceptable for administration of cells to a patient. In some embodiments, the buffer is phosphate buffered saline. In some embodiments, the composition comprises DMSO or glycerol. In some embodiments, the composition is frozen. In some embodiments, the culture media comprises BMP7, FGF2, Heparin, Y27632, LIF, CHIR99021, or combinations thereof.

### Methods of Producing Self-Renewable Nephron Progenitor Cells

Disclosed herein are methods of producing self-renewable nephron progenitor cells (srNPCs) that are capable of generating nephron or kidney structures in vitro or in vivo. In some cases, the srNPCs are produced by culturing cells derived from an embryo, a fetus, or an adult at a specific density, using culture containers with a specific shape, with or without a specific coating, in a defined media containing a mixture comprising growth factors, cytokines, inhibitors, proteoglycans, or combinations thereof.

In some cases, the method of producing a srNPC starts with an embryonic tissue that has been dissociated into individual cells. In some cases, the method of producing a srNPC starts with an intact embryonic tissue. In some cases, the embryonic tissue is an embryonic kidney or other urogenital tissue. In some cases, the method of producing a srNPC starts with a fetal tissue that has been dissociated into individual cells. In some cases, the method of producing a srNPC starts with an intact fetal tissue. In some cases, the fetal tissue is a fetal kidney or other urogenital tissue. In some cases, the method of producing a srNPC starts with a neonatal tissue that has been dissociated into individual cells. In some cases, the method of producing a srNPC starts with an intact neonatal tissue. In some cases, the neonatal tissue is a neonatal kidney or other urogenital tissue. In some cases, the method of producing a srNPC starts with an adult tissue that has been dissociated into individual cells. In some cases, the method of producing a srNPC starts with an intact adult tissue. In some cases, the adult tissue is at least a portion of an adult kidney, menstrual fluid, or other urogenital tissue.

In some cases, the method of producing a srNPC uses tissue from a mammal. In some cases, the mammal is human, mouse, rat, rabbit, guinea pig, dog, cat, cow, pig, goat, horse, monkey, non-human primate, or other mammal. In some cases, the method of producing a srNPC uses tissue from multiple animals or a chimera.

In some cases, the method of producing a srNPC starts with cells that have been selected according to the presence or absence of one or more specific cell surface markers. In some cases, the cell surface molecule comprises SIX2, EpCAM, NGFR, ITGA8, PDGFRA, or combination thereof. In some, cases the cell surface molecule comprises SIX2, EpCAM, NGFR, or combination thereof.

Disclosed herein are defined media for culturing srNPC nephron progenitor self-renewal cells (NPSR) containing a mixture of growth factors, cytokines, inhibitors, proteoglycans, or combinations thereof. In some cases, the defined culture media comprises growth factors. In some cases, the growth factors comprise a bone morphogenic protein (BMP), a fibroblast growth factor (FGF), a WNT pathway growth factor, or combinations thereof. In some cases, the bone morphogenic protein (BMP) comprises BMP7, BMP2, GDF-5, GDF-8, GDF-3, GDF-11, GMP6, BMP-10, or combinations thereof. In some cases, the bone morphogenic protein (BMP) comprises BMP7. In some cases, the fibroblast growth factor (FGF) comprises FGF2, FGF9, FGF20, FGF1, FGF4, FGF19, FGF21, FGF23, or combinations thereof. In some cases, the fibroblast growth factor (FGF) comprises FGF2. In some cases, the WNT pathway growth factor comprises Wnt3, Wnt-3a, Wnt7b, Wnt-5a, or combinations thereof. In some cases, the defined culture media comprises a cytokine. In some cases, the cytokine comprises LIF, IL-6, IL-11, or combinations thereof. In some cases, the cytokine comprises LIF. In some cases, the defined culture media comprises an inhibitor. In some cases, the inhibitor comprises a kinase inhibitor such as an inhibitor of p160ROCK, PKC, PKA, MLCK, or combinations thereof. In some cases, the kinase inhibitor comprises Y-27632. In some cases, the inhibitor comprises a kinase inhibitor such as a glycogen synthase kinase (GSK) inhibitor. In some cases, the GSK inhibitor is a GSK-3, GSK-3α, or GSK-3β inhibitor. In some cases, the GSK-3 inhibitor comprises CHIR99021. In some cases, the defined culture media comprises a proteoglycan. In some cases, the proteoglycan comprises a heparan sulfate. In some cases, the heparan sulfate comprises heparan sulfate proteoglycan 2, heparin, or combinations thereof. In some cases, the heparan sulfate comprises heparin. In some cases, the defined culture media or nephron progenitor self-renewal (NPSR) media comprises BMP7, FGF2, LIF, Y-27632, CHIR99021, heparin, or combinations thereof. In some cases, the defined culture media or nephron progenitor self-renewal (NPSR) media consists of BMP7, FGF2, LIF, Y-27632, CHIR99021, and heparin. In some cases, the components of the growth factor are derived from a mammal. In some cases, the mammal is human, mouse, rat, rabbit, guinea pig, dog, cat, cow, pig, goat, horse, monkey, or other mammal.

In some cases, the method of producing srNPC uses a specified culture dish. In some cases, the culture dish has a U shaped bottom. In some cases, the culture dish has a flat bottom. In some cases, the culture dish is coated with a specific coating that affects cell adherence to the dish. In some cases, the culture dish is coated with matrigel, fibronectin, vitronectin, osteopontin, poly-D-lysine, extracellular matrix, synthetic peptide, Cell-Tak, collagen, laminin, or ultra-low attachment coating. In some cases, the culture dish is uncoated. In some cases, the culture dish is a low adherence dish. In some cases, the culture dish is coated with laminin. In some cases, the culture dish has variable numbers of wells per dish, generally resulting in variable culture volume per well. In some cases, the culture dish has 6 wells, 12 wells, 24, wells, 48 wells, 96 wells, 384 wells, or 1536 wells per dish. In some cases, the culture dish is a 12-well dish. In some cases, the culture dish is a 12-well dish coated with laminin. In some cases, the culture dish is a 96-well dish. In some cases, the culture dish is a 96 U shaped well dish with a low attachment coating. In some cases, the culture dish is a 35 mm, dish, a 60 mm dish, 100 mm dish, a 150 mm dish, or a smaller or larger culture dish known by a person in the art. In some cases, the culture dish is a flask, such as a 25 cm² flask, a 75 cm² flask, a 175 cm² flask, a 300 cm² flask, or a smaller or larger culture flask known by a person in the art.

In some cases, method of producing srNPCs involves culturing the starting material (e.g., cells or tissues from embryonic, fetal, neonatal or adult sources) at a specific density of cells per size of a culture dish. In some cases, the cells are cultured at a density of 100-200, 200-500, 500-1000, 1000-5000, 5000-10000, 10000-50000, 500000-100000 cells per well of a 96-well dish, or equivalent density in a smaller or larger culture dish. In some cases, the cells are cultured at a density of 5000 cells per well of a 96-well dish. In some cases, the cells are cultured at a density of 10000 cells per well of a 96-well dish. In some cases, the cells are cultured at a density of 5000-10000 cells per well of a 12-well dish.

In some cases, the method of producing srNPCs involves collecting the cells that have aggregated and float in the culture dish (i.e., aggregated, floating cells) after culturing the starting material. In some cases, the aggregated, floating cells comprise an aggregate that is about 1 mm in diameter. In some cases, the aggregate is 0.1-5 mm in diameter. In some cases, the aggregated, floating cells are collected 1-10 days after culturing the starting material. For example, floating cells are collected, 1, 2, 3, 4, 5, 6, 7, 8, 9, 10, or more days after culturing the starting material. In some cases, the aggregated, floating cells are collected 4-6 days after culturing the starting material. In some cases, the aggregated, floating cells are collected 5-7 days after culturing the starting material. In some cases, the aggregated floating cells are SIX2+.

### Methods of Producing Fate-Specified Nephron Progenitor Cells

Disclosed herein are methods of producing fate-specified nephron progenitor cells (FS-NPCs). As described herein, FS-NPCs are committed srNPCs that are capable of differentiating to form nephron organoids. In some cases, the method of producing FS-NPCs comprises contacting srNPCs with one or more factors convert a srNPC to a FS-NPC. In some cases, the FS-NPCs are produced by contacting a srNPC with a spinal tissue, such as a portion of a spinal cord, cells from a spinal cord, or a spinal cord extract. In some cases, the spinal tissue is derived from the same source as the srNPCs, for example a mouse srNPC and a mouse spinal tissue. In some cases, the spinal tissue is from a different source as the srNPCs, for example, a human srNPC and a mouse, or other mammalian spinal tissue capable of converting the srNPC to the FS-NPC. In some cases, the FS-NPCs are produced by contacting a srNPC with a culture media containing one or more growth factors, inhibitors, or combinations thereof. In some cases, the growth factor is a fibroblast growth factor (FGF) such as a FGF2, FGF9, FGF20, FGF1, FGF4, FGF19, FGF21, FGF23, or combinations thereof. In some cases, the inhibitor is a glycogen synthase kinase (GSK) inhibitor. In some cases, the GSK inhibitor is a GSK-3, GSK-3α, or GSK-3β inhibitor. In some cases, the GSK-3 inhibitor comprises CHIR99021. In some cases, the FS-NPCs are produced by contacting a srNPC with a mixture comprising FGF2, CHIR99021 or a combination thereof. In some cases, the FS-NPCs are produced by contacting a srNPC with a mixture consisting or consisting essentially of FGF2 and CHIR99021.

### Methods of Treatment

Disclosed herein are methods of treating a subject in need thereof comprising administration of a srNPC or FS-NPC described herein that have been created using methods described herein in an amount sufficient to treat the kidney disease. In some cases, the srNPC or FS-NPC is derived from a specific individual, such as the individual in need of treatment or an individual having a specific genetic makeup, thereby allowing the methods of treatment to be tailored to each individual. In some cases, the subject in need of treatment is experiencing acute or chronic kidney disease. In some cases, treating the kidney disease cures the kidney disease. In some cases, treating the kidney disease reduces symptoms or complications caused by the kidney disease. In some cases, treating the kidney disease improves renal function. In some cases, treating the kidney disease delays, reduces, or eliminates the need for a kidney transplant. In some cases, treating the kidney disease improves the prognosis or quality of life of the subject.

Disclosed herein are methods of treating a subject with kidney disease comprising administration of a srNPC or FS-NPC in an amount sufficient to treat the kidney disease. In some cases, the kidney disease is acute. In some cases, the kidney disease is chronic. In some cases, the kidney disease comprises nephritis, analgesic nephropathy, diabetic nephropathy, IgA nephropathy, iodinated contrast media induced nephropathy, lithium induced nephropathy, xanthine oxidase deficiency, polycystic kidney disease, or onconephropathy. In some cases, the kidney disease has led to renal insufficiency, renal failure, and/or uremia.

In some cases, kidney disease leads to renal failure, kidney failure, or renal insufficiency. In some cases, renal failure occurs when the kidneys fail to adequately filter waste products from the blood. In some cases, renal failure is reversible (i.e., acute). In some cases, renal failure is irreversible (i.e., chronic). In some cases, renal failure is determined by a decrease in glomerular filtration rate (e.g., the rate at which blood is filtered in the glomeruli of the kidney). In some cases, renal failure is detected by a decrease or absence of urine or an increase in waste products (e.g., creatinine or urea) in the blood. In some cases, renal failure is detected by blood or serum proteins in the urine. In some cases, renal failure is detected by increased fluid in the body, increased acid levels, increased potassium levels, decreased calcium levels, increased phosphate levels, or anemia. Additional symptoms of kidney failure include but are not limited to vomiting, diarrhea, dehydration, nausea, weight loss, nocturnal urination, change in frequency of urination, blood in urine, pressure or difficulty in urinating, change in volume of urination, itching, bone damage, nonunion of broken bones, muscle cramps, abnormal heart rhythm, muscle paralysis, swelling, shortness of breath, pain, fatigue, memory problems, dizziness, loss of appetite, difficulty sleeping, or darkening of the skin.

In some cases, the srNPCs or FS-NPCs are administered to the subject in need thereof in an amount sufficient to treat the kidney disease. In some cases, the srNPC or FS-NPC are administered via routes of administration known in the art. Routes of administration include but are not limited to cutaneously, intraarterially, intradermally, intraductally, intradermally, intralesionally, intraluminally, intramuscularly, intraperitoneally, intratubularly, intravenously, parenterally, subcutaneously, or other method known by one of skill in the art. The amount of srNPC or FS-NPC administered is known by one of skill in the art and may depend on the size of the subject and the severity of the disease to be treated. In some cases, 1000-1000000 or more srNPC or FS-NPC are administered. In some cases, 1000-2000, 2000-5000, 5000-10000, 10000-30000, 30000-100000, 100000-500000, 500000-1000000, or more srNPC or FS-NPC are administered. In some cases, the srNPC or FS-NPC are administered daily, weekly, bi-weekly, monthly, every three months, every six months, or yearly. In some cases, a single administration of srNPC or FS-NPC is required for treatment of the subject. In some cases, the srNPC or FS-NPC are administered in combination with another treatment for kidney disease such as dialysis. In some cases, the srNPC or FS-NPC are administered in combination with a drug that treats or prevents rejection of the srNPC or FS-NPC by the immune system of the subject.

Methods of treatment disclosed herein comprise treating a subject with kidney disease. In some cases, the subject is a mammal such as a human, a dog, a cat, a rabbit, a mouse, a guinea pig, a horse, a cow, a pig, a goat, a monkey, a non-human primate, or other mammal in need of treatment of kidney disease.

### Methods of Drug Toxicity Screening

Disclosed herein are methods of screening a drug for toxicity comprising contacting the drug to a srNPC or FS-NPC and detecting signs of toxicity in the srNPC or FS-NPC. In some cases, signs of toxicity include but are not limited to changes in glomerulus morphology, cellular apoptosis, or combinations thereof. In some cases, changes in glomerulus morphology include but are not limited to podocyte injury such as focal glomerular segmental sclerosis, capsular adhesion, crescent formation, and foot process effacement. In some cases, cellular apoptosis or toxicity is detected by methods including but is not limited to gamma-H2AX staining or caspase staining, including caspase-3 staining.

Also disclosed herein are methods of determining the susceptibility of an individual to drug toxicity to one or more drugs comprising preparing a srNPC or FS-NPC from tissues derived from the individual, contacting one or more drugs to the srNPC or FS-NPC, and detecting signs of toxicity in the srNPC or FS-NPC. In some cases, signs of toxicity include but are not limited to changes in glomerulus morphology, cellular apoptosis, or combinations thereof. In some cases, changes in glomerulus morphology include but are not limited to podocyte injury such as focal glomerular segmental sclerosis, capsular adhesion, crescent formation, and foot process effacement. In some cases, cellular apoptosis or toxicity is detected by methods including but not limited to gamma-H2AX staining or caspase staining, including caspase-3 staining.

### Discussion of Experimental Results

Disclosed herein is a robust strategy for the isolation, cultivation and differentiation of early NPCs in physiological 3D culture conditions. srNPCs cultured in a chemically defined NPSR medium contain a highly pure NPC-like population displaying full developmental potential in vivo. Through a differentiation trajectory that closely resembles in vivo nephrogenesis, srNPCs can generate renal organoids in a rapid and efficient manner. An immediate next derivative of srNPCs, FS-NPCs, exhibit niche-independent differentiation features rendering them amenable for adult kidney repair where de novo nephrogenesis is lost. The successful derivation of human srNPCs opens new avenues for studying human kidney development, renal disease modeling, and cell-replacement therapies.

### Aggregate Culture Facilitates Self-Renewal of Mammalian NPCs

In situations where monolayer cultures are not able to reproduce proper in vivo microenvironments, 3D aggregate cultures may constitute an alternative option for in vitro differentiation protocols. A few prime examples include the induction of primordial germ cell-like cells (PGC-LCs) from mouse and human PSCs as well as kidney metanephric mesenchyme differentiation from PSCs. In trying to mimic in vivo tissue architecture, 3D cultures have also been used recently for the generation of organoids. Disclosed herein is a novel 3D aggregate culture that facilitates long-term self-renewal of mouse and human nephron progenitors. Culturing primary NPCs in monolayer conditions results in adherent NPCs that grow slowly and undergo a gradual upregulation in E-cadherin expression, a MET marker indicative of further differentiation. Eventually 2D cultured NPCs lose their mesenchymal identity and nephrogenic potential. Other limitations associated with monolayer NPEM culture include: 1) the limited utility for expanding NPCs from developmental stages prior to E13.5 and after P1. 2) NPCs cultured in NPEM are prone to spontaneous differentiation. 3) In vivo developmental potential of NPEM cultured NPCs are not demonstrated. Advantages of 3D aggregate culture include: 1) derivation of NPC lines from wide kidney developmental stages (E11.5 to P1); 2) an increase in derivation efficiency, up to 100%; 3) stable and long term maintenance of NPC lines in culture with no signs of differentiation; 4) the development and successful application of stringent in vivo NPC functional tests.

In some cases, 3D culture methodologies such as bioreactors can be exploited for large-scale cell production. The culture method presented herein holds the potential for development of scalable NPC culture technologies towards the implementation in the kidney field of new drug toxicity screening assays and cell-transplantation therapies where large amount of cells of molecular and functional purity is mandatory. As with mesenchymal stem cells (MSCs), distinct cellular morphologies and mechanophysical properties were observed between 3D and 2D cultures and these differences not only contributed to altered gene expression but also differentiation propensity.

### A Chemically Defined Robust NPSR Culture Condition

Although necessary, 3D culture alone is not sufficient for the long term self-renewal of isolated NPCs. Through screening for a selected list of growth factors and small molecules, a minimal cocktail of factors has been determined that, when combined with 3D aggregate culture (NPSR medium), are sufficient for propagating functional NPCs in vitro. Specific modulators of several signaling pathways including BMP, FGF, and WNT, play key roles in NPCs self-renewal, survival as well as differentiation are included in NPSR medium. The loss of Six2-GFP signal after exposure to a given concentration of CHIR led us to uncover a CHIR concentration dependent sensitivity of NPCs. Additionally, the culture conditions herein shed new light on the role LIF plays in maintaining NPC identity in addition to its well-known function in regulating MET transition and inducing nephrogenesis. It should be noted that withdrawal of any component in the NPSR medium compromised the self-renewal, potency, or survival of srNPCs. Thus the chemically defined NPSR culture herein provides a minimal synthetic niche environment for the robust propagation of pure functional NPCs. This not only serves as the basis for gaining mechanistic insights into the signaling networks underlying NPCs self-renewal and stemness, but also constitutes a starting point for understanding the genetic and epigenetic events regulating NPC differentiation.

srNPCs Resemble Early NPCs in Development

Mammalian NPCs exist within a time window stretching from E11.5 to P3 in vivo. RNA-seq analysis of NPCs from different developmental stages revealed distinct gene expression profiles shared between early (E11.5-E13.5) and late (E16.5 and P1) NPCs. These molecular discrepancies may be caused by either stage-specific cell-intrinsic cues or different extrinsic niche environmental signals that differ in early versus late developing kidneys. The NPSR culture herein is able to converge NPCs from all examined stages (E11.5, E12.5, E13.5, E16.5 and P1) into a common self-renewal state with global gene expression closely resembling that of early NPCs. This likely can be explained by two possibilities: NPCs from late developmental stages may contain heterogeneous populations and the NPSR culture conditions may selectively promote a specific population of cells that resemble early NPCs; an alternative explanation is that late stage NPCs are plastic and the NPSR culture facilitates reversion of late NPCs to an early stage in vitro.

srNPCs Exhibit Full Developmental Potential and Adult Kidney Repair Capabilities

Functionally speaking, srNPCs could give rise to derivatives and structures representative of an intact nephron. This was demonstrated herein by using a well-established in vitro spinal cord induction assay and ex vivo transplantation. Another functional criterion would be an in vivo transplantation assay where both host organism and grafted cells are developmentally synchronized in time. Considering that srNPCs resemble early NPCs (E11.5-E13.5), and although large animals such as pig or goat could be envisioned in the near term, currently it is technically challenging to perform in utero injections into a timely matched mouse embryonic niche. Injection of srNPCs to neonatal kidney cortex didn't lead to their differentiation, highlighting a niche-dependent property of srNPCs. Thus, and to demonstrate the range of different cell types that srNPCs could become, a well-established developmental potential assay that utilizes the chick embryo as a host was used. Direct injection of srNPCs into the coelomic cavity of a developing chick embryo provides a permissive environment conducive to generate an entire nephron in vivo, thus, unequivocally demonstrating the full developmental potential of mammalian srNPCs.

An immediate next derivative of srNPCs, FS-NPCs, was able to spontaneously differentiate into all nephron structures, both in vitro and in vivo in the post-embryonic kidney cortex. The niche-independent features of FS-NPCs could be further extended into the adult kidney where embryonic de novo nephrogenesis signals are no longer present. Additionally, FS-NPCs could induce ectopic mini-kidneys containing urine-like fluids upon transplantation to NSG mice. Together these observations raised the intriguing possibility of using FS-NPCs for adult kidney repair. To this end, the regenerative potential of FS-NPCs was tested in an acute kidney injury model. Upon transplantation to the renal subcapsule, FS-NPCs could generate different portions of the nephron and more importantly, significantly improved renal function as evidenced by normalization of different kidney specific biochemical parameters including lowered blood BUN and serum creatinine values. And as a result, the survival of cisplatin-treated NSG mice was extended.

While all stem cells and progenitors based kidney research remain mainly experimental, the stability, homogeneity, and full-functionality conferred by the 3D culture here reported may facilitate srNPCs early venturing into pre-clinical and clinical testing. The specific nature and stage of renal disease will eventually determine the amenability of FS-NPCs for any putative therapeutic intervention. While the results herein highlight the usefulness of FS-NPCs in an acute kidney injury model, future endeavors to identify early diagnostic markers for chronic kidney conditions may facilitate new cellular interventions rooted in the use of pluripotent stem cell derivatives, embryonic progenitors, and adult stem cells.

srNPC-Based Disease Modeling and Drug Screening Platforms

The methodologies herein introduced represent a first proof of concept for the derivation of srNPC lines from both wild type as well as transgenic mouse strains. In some cases, the CDP method could be easily adapted to hundreds of transgenic mice lines already generated and available worldwide. When combined with state-of-art nuclease-based gene editing technologies such as the CRISPR-Cas9 technology, this platform could be further expanded and enhanced by genetically manipulating srNPCs, including generation of newer kidney specific knock-out and knock-in diseased model lines as well as gene-corrected lines. Additionally, the efficient and rapid differentiation of srNPCs into 3D nephron organoids resembling normal kidney morphogenesis may lead to the prospective development of a high-throughput compound screening platform to address issues related to kidney drug toxicity, efficacy, and safety. Key major obstacles that have constrained the rapid advance of both fundamental biology as well as translational kidney research include the limited availability of primary NPCs, the time and labor-intensive generation of transgenic animal models, and the lack of robust 3D renal cultures. Observations disclosed herein may help to address some of these challenges, and in so doing, promote the conceptualization and development of the different platforms here envisioned with the goal of enhancing current knowledge of mammalian and especially human kidney morphogenesis, modeling of kidney-related human diseases, diagnostics, and eventually cell-replacement therapies.

### Definitions

A “nephron progenitor cell” or a “NPC” used interchangeably herein, comprises a self-renewing stem cell population capable of giving rise to nephrons, the functional unit of the kidney.

A “self-renewable nephron progenitor cell” or a “srNPC”, used interchangeably herein, comprises a nephron progenitor cell derived from an embryonic, fetal, neonatal, or adult tissue by methods described herein and retains the capability of unlimited or nearly unlimited cell expansion. The srNPC is also capable of in vitro or in vivo differentiation into nephron or kidney structures.

A “fate-specified NPC” or a “FS-NPC”, used interchangeably herein, comprises a committed srNPC with the ability to form nephron organoids in vivo or in vitro. In some embodiments, the FS-NPC is derived from a srNPC by methods described herein.

“Nephron progenitor self-renewal media”, “NPSR”, or “defined media”, used interchangeably herein comprises a defined culture media for use in the methods of producing srNPC described herein. In some embodiments, NPSR comprises a mixture of growth factors, cytokines, inhibitors, proteoglycans, or combinations thereof. In some embodiments, NPSR comprises one or more of BMP7, FGF2, Heparin, Y27632, LIF, and CHIR99021.

“LIF” refers to leukemia inhibitory factor protein, is an interleukin 6 class cytokine. In some embodiments, LIF is a mammalian protein. In some embodiments, LIF is a human protein. In some embodiments, LIF is a mouse protein.

“Kidney disease”, “renal insufficiency”, “renal failure”, or “uremia”, used interchangeably herein, comprises acute or chronic dysfunction of one or more kidneys in a subject. Kidney disease includes but is not limited to nephritis, analgesic nephropathy, diabetic nephropathy, IgA nephropathy, iodinated contrast media induced nephropathy, lithium induced nephropathy, xanthine oxidase deficiency, polycystic kidney disease, or onconephropathy.

“About” as used herein in reference to a number refers to that number plus or minus 10%.

### EXAMPLES

The following examples are given for the purpose of illustrating various embodiments of the invention and are not meant to limit the present invention in any fashion. The present examples, along with the methods described herein are presently representative of preferred embodiments, are exemplary, and are not intended as limitations on the scope of the invention. Changes therein and other uses which are encompassed within the spirit of the invention as defined by the scope of the claims will occur to those skilled in the art.

### Example 1: Derivation and Long-Term Culture of Murine srNPCs

NPCs in mouse first appear around embryonic day 11.5 (E11.5) and mark the onset of nephrogenesis. In mice, NPCs exhaust shortly after birth, around postnatal day 3 (P3), after their last wave of differentiation into nephrons. Lineage tracing experiments have established SIX2 as a specific NPC marker. Available mouse strain with GFP cassette knocked in the Six2 locus (Six2^{GCE}) facilitates purification of Six2-GFP+ population from embryonic or neonatal mouse kidneys by fluorescence activated cell sorting (FACS). To screen for conditions conducive to in vitro expansion of NPCs Six2-GFP+ cells were isolated from E13.5 embryonic kidneys of Six2^{GCE} mice. Considering that in vivo NPCs are tightly packed together, it was hypothesized that the cell-cell contact might play a role in their survival and proliferation. Thus, to better preserve the native microenvironment, sorted Six2-GFP+ NPCs were transferred into U-bottom low-attachment 96-well plates to form 3D aggregates. In base medium supplemented with serum alone NPC aggregates could form. GFP signal was observed within the first 12 hours but decreased upon further culture and eventually lost in 48 hours, indicating that Six2 expression in NPCs could not be maintained in the absence of proper extrinsic signals recapitulating the NPC in vivo niche.

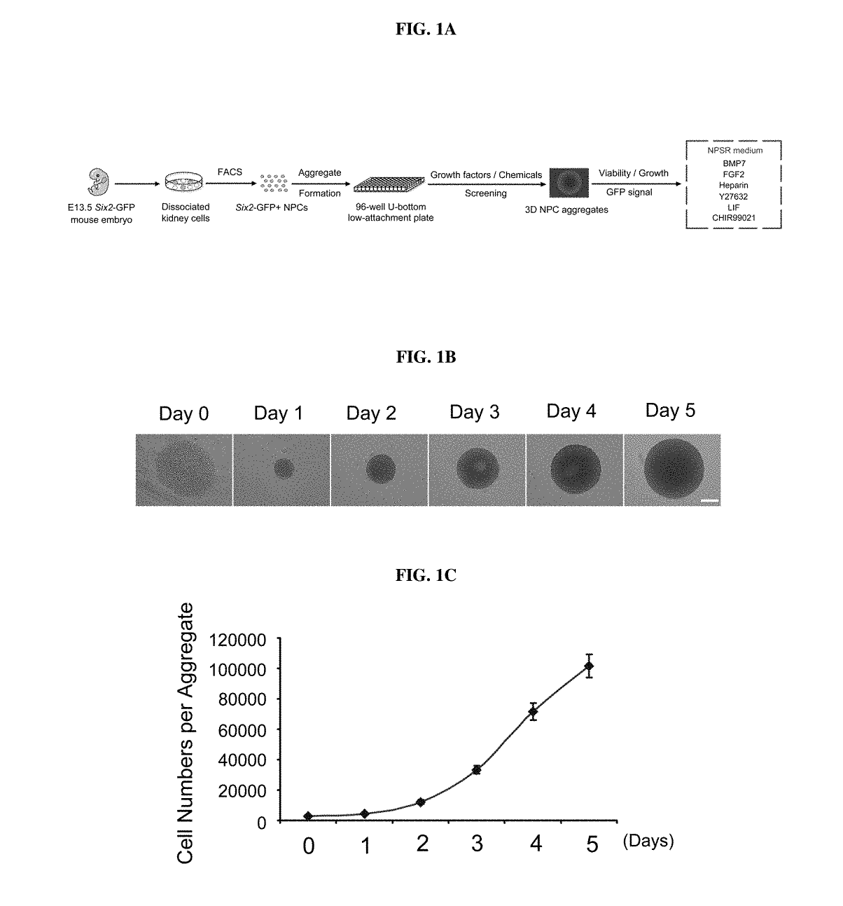

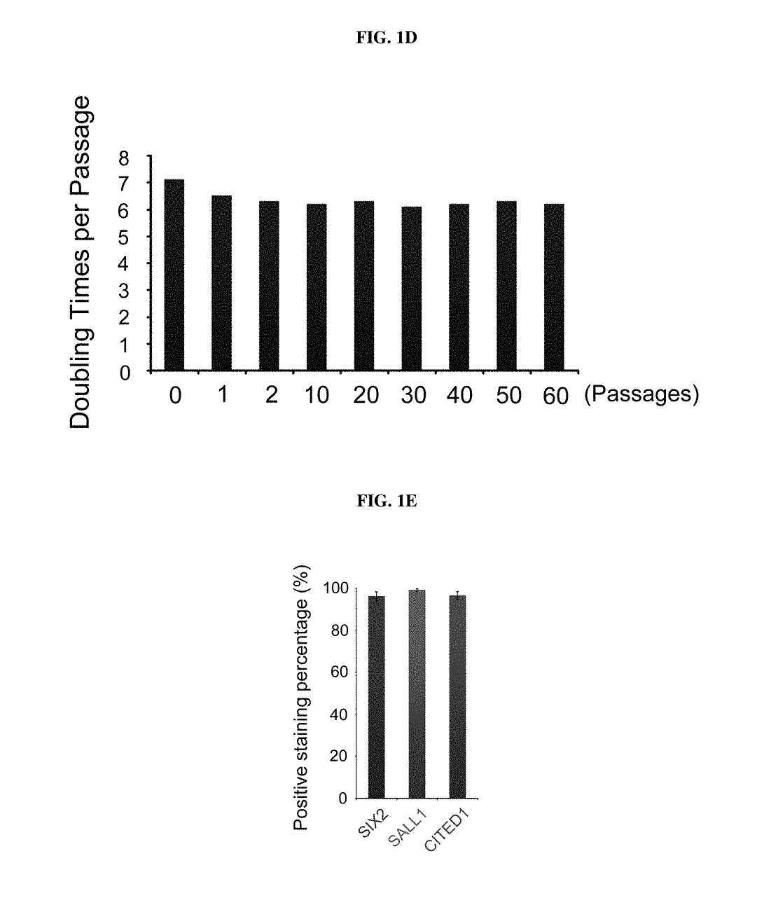

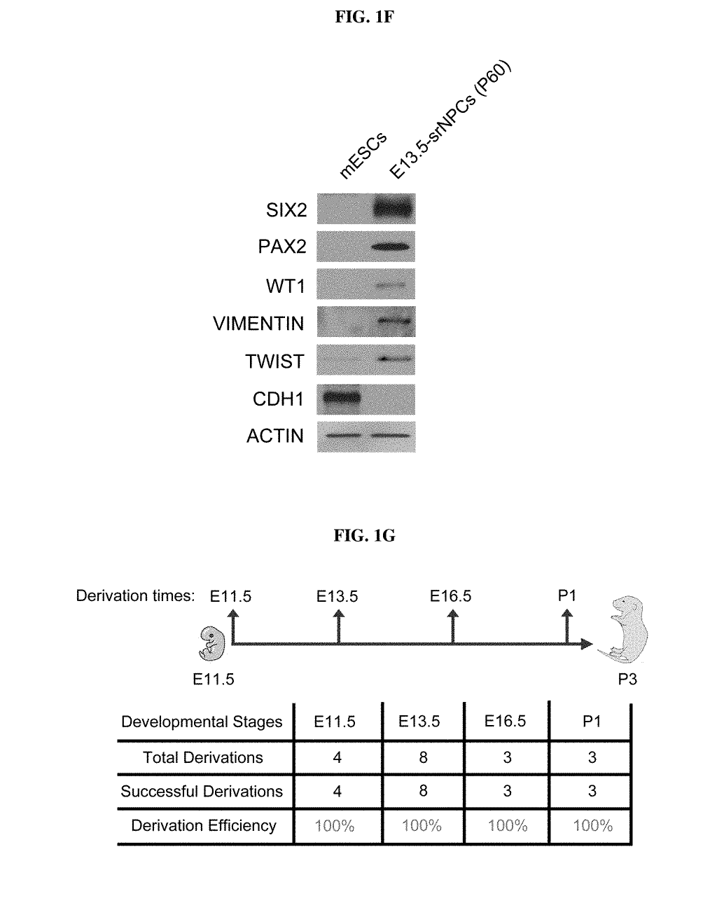

The optimal condition supportive of NPC self-renewal in vitro was determined by screening growth factors and chemical inhibitors for known signaling pathways implicated in regulating NPC survival, self-renewal, and differentiation in vivo as well as other signaling pathways that were not previously reported. Cell viability, growth rate, and GFP signal were considered among other criteria. Through successive testing a novel culture condition was established containing a cocktail of BMP7, FGF2, Heparin, Y27632, LIF, and CHIR99021 that promotes long-term in vitro expansion of Six2-GFP+ NPCs with high purity (it was named NPC self-renewal medium, or NPSR medium) (FIG. 1A). Of note is that isolated Six2+ NPCs have been propagated in this culture for more than one year and more than 70 passages. The established NPC lines were designated as self-renewable NPCs (srNPCs) due to their unlimited proliferative potential. srNPCs grow fast under this culture condition. Starting with 3,000 cells, after 5 days, the aggregate can grow to around 1 mm in diameter containing around 100,000 cells (FIG. 1B and FIG. 1C). srNPC aggregates maintain stable growth rate after long term passages with 6 to 7 doublings per passage (FIG. 1D). Importantly, after 60 passages, the GFP+ cell percentage in srNPCs was quantified and found that 98.44% of the srNPCs remained GFP+, with the GFP signal intensities similar to that of freshly isolated primary NPCs. Moreover, similar results were obtained when SIX2 and other NPC marker gene expressions were quantified by immunostaining: SIX2+ (˜96.2%), SALL1+ (˜99.1%), CITED1+ (˜96.1%) (FIG. 1E). These results indicate that highly pure srNPCs with homogenous expression of NPC marker genes can be maintained long-term in the novel 3D culture conditions. The expression of TWIST and VIMENTIN, but not E-CADHERIN in srNPCs further confirms their mesenchymal identity (FIG. 1F). Next it was investigated whether all components are needed for sustaining robust expansion of srNPC. To this end each of the six components were subtracted from NPSR medium and evaluated the effects on srNPCs. It was found that all six components were indispensible for optimal propagation of srNPCs in vitro. Removal of each component for a period as short as 4 days resulted in slower cell growth (withdrawal of FGF2, Heparin, or Y27632), loss of GFP signal (withdrawal of LIF), or both (withdrawal of BMP7 or CHIR99021).

It was determined whether NPSR culture could also support the derivation of srNPCs from other developmental stages harboring Six2+ populations. To this end, additional time points were selected including E11.5, E16.5 and P1. Remarkably, srNPCs could be successfully derived from all stages with 100% efficiency. (FIG. 1G) Interestingly, a slower growth rate was observed in the first three passages of P1-derived srNPCs. Upon further passages, P1-srNPCs acquired similar growth kinetics and were indistinguishable from srNPCs derived from earlier developmental stages. Similarly to E13.5-derived srNPCs, all E11.5, E16.5 and P1-derived srNPCs showed highly homogeneous GFP signal and NPC marker gene expression.

RNA-seq analysis was performed of srNPCs and their cognate in vivo NPCs from different developmental stages. For controls, Six2-GFP− and Six2-GFP+ cells were sorted from E12.5 Six2^{GCE} mouse embryonic kidneys and subjected to RNA sequencing. 1218 differentially expressed genes were found between Six2-GFP− and Six2-GFP+ cells which were subsequently used as NPC signature genes for further analysis (Table 1). Principal component analysis (PCA) of the NPC signature genes revealed that primary NPCs are separated into two discrete groups according to their developmental stages: E11.5, E12.5 and E13.5 were clustered together indicative of an early NPC identity while E16.5 and P1 were found in the late NPCs group. Interestingly, regardless of timing of derivation, all srNPCs are clustered together in a group much closer to the early rather than late NPCs on both PC1 and PC3 axes. Compared to primary NPCs, srNPCs express virtually all the known NPC marker genes at similar levels, while UB and stromal progenitor cells specific genes are kept at similar low or even lower levels. Also, two well-established NPC surface markers ITGA8+PDGFRA− were maintained in the srNPCs. These data indicate that NPSR culture conditions corralled Six2-GFP+ cells from different developmental stages into self-renewal in a cellular state manifesting key molecular features characteristic of primary early-stage NPCs.

### Example 2: srNPCs Retain Full Nephrogenic Potential

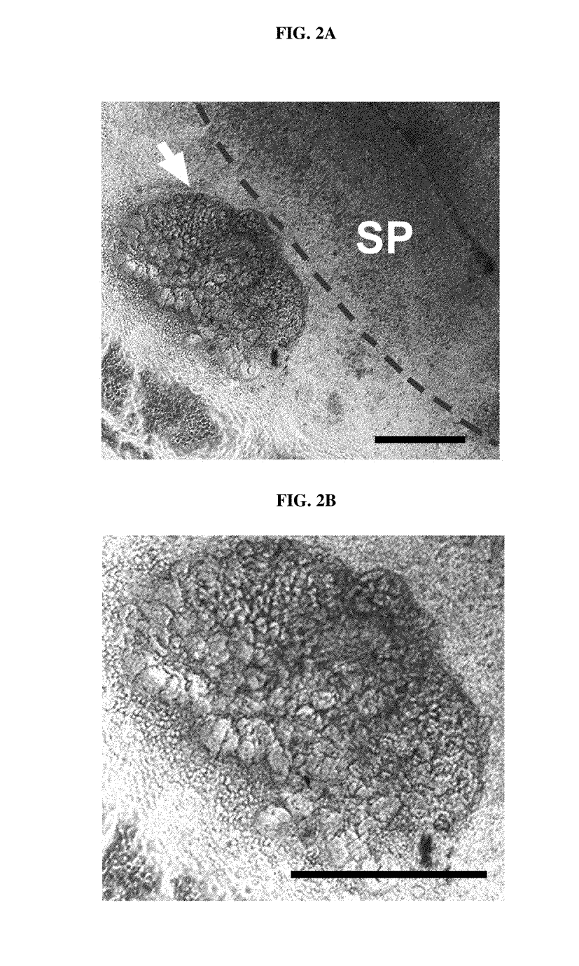

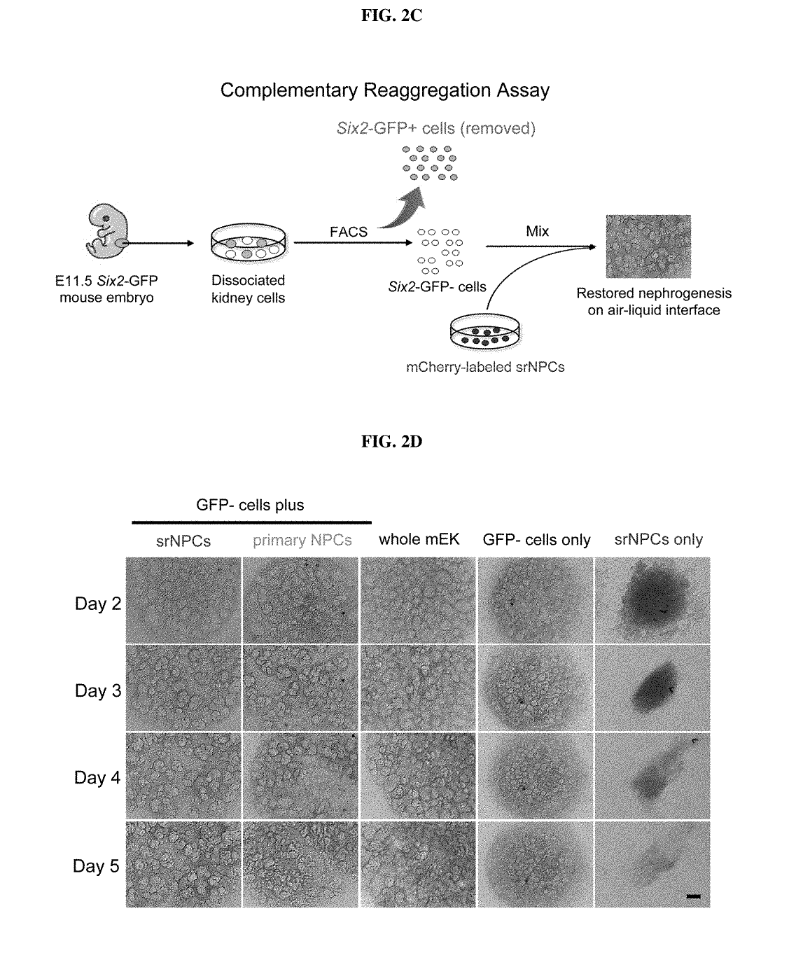

An embryonic dorsal spinal cord induction assay was used to test the nephrogenic potential of long-term cultured srNPCs. Long-term cultured E13.5-derived srNPCs were co-cultured with embryonic dorsal spinal cord in an air-liquid interface. Many tubular structures were observed to form on day 3 and their number dramatically increased from day 3 to day 7 (FIG. 2A and FIG. 2B). On day 7, the generated tubular structures were examined by immunocytochemistry. Positive staining was observed for glomerulus markers Podocalyxin (PODXL), Synaptopodin (SYNPO), and Wilms Tumor 1 (WT1); proximal tubule markers Lotus Tetragonolobus Lectin (LTL) and Aquaporin 1 (AQP1); distal tubule markers E-Cadherin (CDH1), Pax2, and Dolichos Biflorus Agglutinin (DBA), suggesting proper differentiation of srNPCs to all three major segments of nephrons. Similarly, srNPCs derived from E11.5, E16.5 and P1 stages exhibited similar differentiation potential upon induction. These data indicate that, provided with appropriate inductive signals, srNPCs could differentiate into nephron structures in vitro.

During normal kidney development, reciprocal interactions between ureteric bud (UB) tips and NPCs underlie kidney morphogenesis. To test if the srNPCs could interact with UB and commit to nephron formation, a complementary reaggregation assay was employed by mixing srNPCs with only UB fractions of embryonic kidney. To this end, E11.5 embryonic kidney was dissociated from Six2^{GCE} mice and the Six2-GFP+ NPCs population depleted by FACS. Remaining Six2-GFP− populations (mostly composed of UB cells) were mixed with mCherry-labeled srNPCs to form aggregates and then transferred to air-liquid interface to test if nephrogenesis could occur ex vivo (FIG. 2C). As positive controls, Six2-GFP− cells were mixed with primary Six2-GFP+ NPCs, or dissociated unfractionated E11.5 embryonic kidney cells were used. As shown in FIG. 2D, time-lapse imaging revealed a dynamic nephrogenic process following mixing primary Six2-GFP− cells with mCherry-labeled srNPCs, which was indistinguishable from what was observed with two positive controls. As expected, Six2-GFP− cells alone only formed tubules while massive cell death was observed in the srNPCs alone group. UB-MM interactions are marked by the formation of a condensed cap structure known as cap mesenchyme, which is composed exclusively of nephron progenitors, surrounding each UB tip. In day 2 reaggregates, most if not all mCherry-labeled srNPCs were found to be located to the cap structures surrounding UB tips Immunofluorescence analysis confirmed SIX2 expression in mCherry+ cells in all cap structures examined. Furthermore, following a 7-day course of morphogenesis, aggregates at day 7 were marked by the presence of numerous mCherry+ glomerulus structures as evidenced by positive PODXL and WT1 staining Collectively, these data indicate that srNPCs are responsive to inductive signals emanating from primary UB cells and can initiate a cascade of organogenic events conducive to the generation of nephrons in an ex vivo setting.

### Example 3: Robust and Efficient Generation of Nephron Organoids from srNPCs

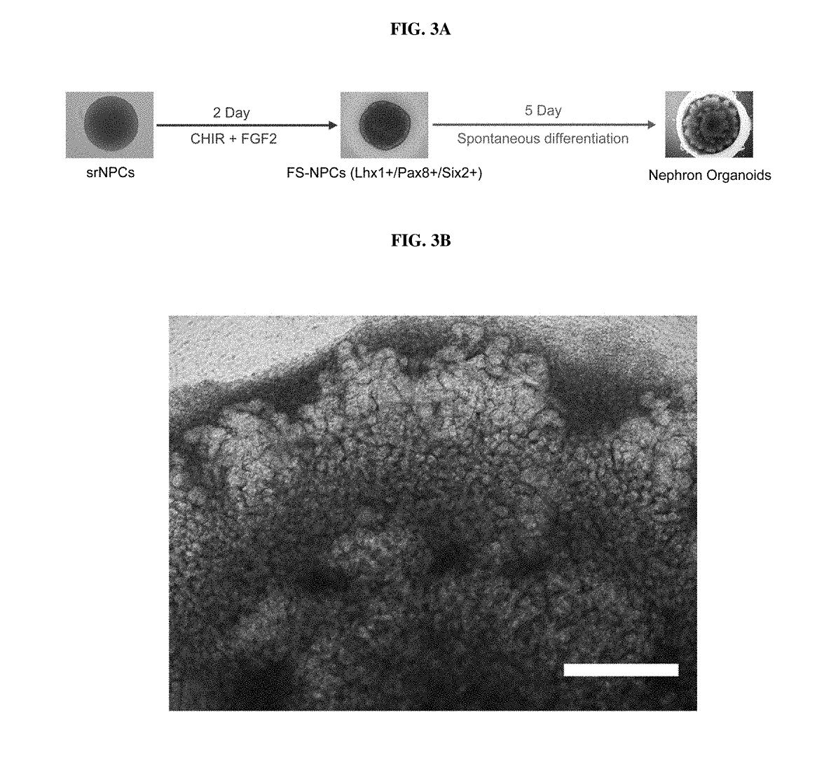

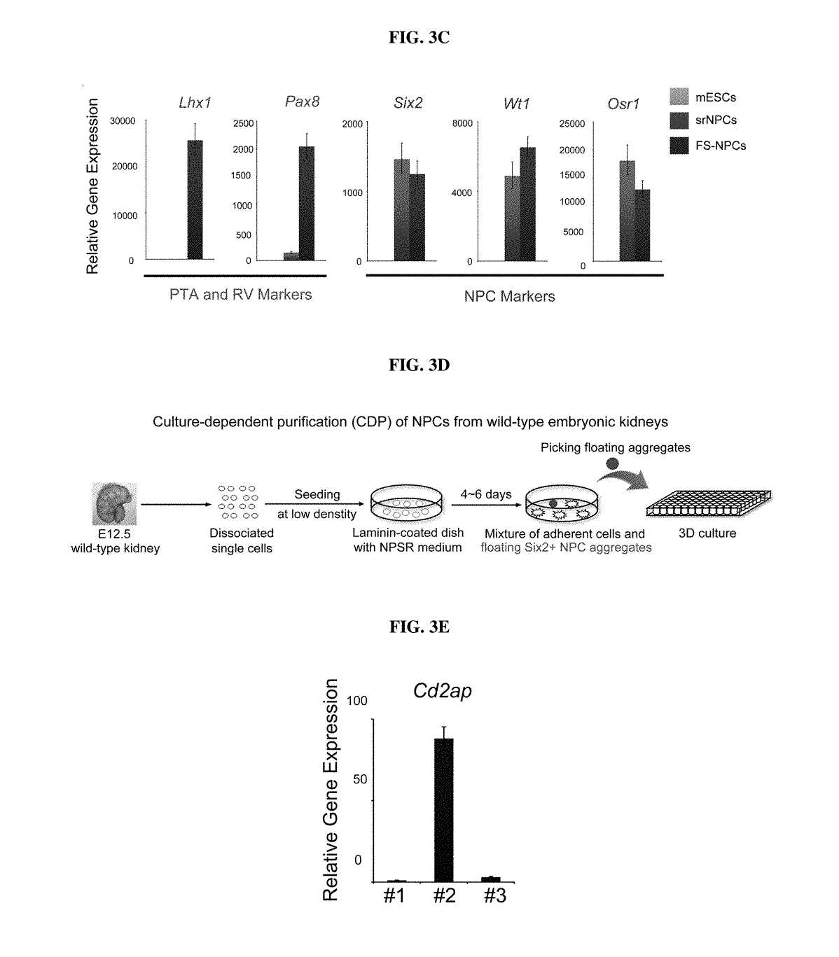

Given kidney's primary function in plasma filtration and metabolic homeostasis, drug-induced nephrotoxicity is common and constitutes a major consideration during the development of new therapeutic drugs. Currently, drug toxicity screening is largely performed using monolayer cultured immortalized kidney cells. Cells in monolayer culture, however, lack an intact 3D nephron structure and are thus limited for accurate prediction of a drug's effect on kidney function. Another layer of complexity during drug screening using immortalized cell lines relates to genetic abnormalities. In the spinal cord induction assay it was found that a minimal two-day induction was sufficient for srNPC to commit to nephrogenesis as evidenced by autonomous nephron organoids formation after separation from the spinal cord. A screen was done to determine conditions that allow efficient organoid generation from srNPCs. It was found that stimulating srNPCs with CHIR99021 and FGF2 for two days could faithfully reproduce the nephrogenesis commitment induced by the spinal cord (FIG. 3A). Committed srNPCs were designated as fate-specified NPCs or FS-NPCs. FS-NPCs could further differentiate autonomously and form nephron organoids marked by numerous tubular formations (FIG. 3B). Whole-mount staining of obtained nephron organoids identified numerous PODXL+/WT1+ glomeruli, LTL+ proximal tubules and CDH1+ distal tubules. The transition from srNPCs via FS-NPCs to nephron organoids was rapid (as short as 7 days), highly efficient and composed of all major nephron segments representing the glomeruli and proximal and distal tubules.

In vivo, NPCs first differentiate to pretubular aggregates (PTA) and renal vesicles (RV) before further maturation to form the segmented renal tubules. To determine which stage FS-NPCs likely correspond to, the expression of common PTA and RV markers was checked. Quantitative PCR (qPCR) analysis indicated that PTA and RV markers Lhx1 and Pax8 were strongly upregulated while NPC markers including Six2, Wt1, and Osr1 were maintained at similar levels to srNPCs (FIG. 3C). Immunofluorescence studies confirmed protein expression of both PTA/RV markers (LHX1 and PAX8) and NPC markers (SIX2 and SALL1) in FS-NPCs. These results suggest that FS-NPCs are closer to the PTA than to the RV stage. To further investigate to what degree the nephron organoid formation process mimics a normal nephrogenesis process after transitioning to the PTA-like stage (FS-NPC), from day 2 to day 7 a DAPT was used to inhibit Notch signaling, which has been shown to be necessary for proximal tubules and glomerular podocytes formation. Upon DAPT treatment, both proximal tubules and glomerulus differentiation were inhibited. This further shows that srNPC-based nephron organoid formation can recapitulate the processes taking place during in vivo nephrogenesis.

The utility of srNPC-derived organoids for renal toxicity testing was determined. To this end, day 7 organoids were treated with lipopolysaccharide (LPS), which can elicit injuries in the glomerulus. After two-day exposure to LPS, nephron organoids displayed specific damages in the glomerulus as evidenced by disrupted glomerulus morphology and active Caspase 3 (CASP3) staining for apoptotic cells. Combined, these results highlight the robustness and efficiency of nephron organoid formation from srNPCs in a manner recapitulating normal nephrogenesis in vivo and the suitability of these in vitro generated organoids for renal toxicity testing.

### Example 4: srNPCs are Amenable to Gene-Editing and Disease Modeling

Over the last two decades, hundreds of mouse strains harboring gene modifications useful for studying kidney development or modeling human genetic kidney diseases have been generated. Efficient srNPC derivation and organoid formation methodologies disclosed herein could be further expanded into existing genetic models for a better understanding of kidney morphogenesis as well as disease progression. Towards achieving these goals, a srNPC derivation independent from Six2 reporters was developed. A culture-dependent purification (CDP) method was developed for the derivation of srNPC lines from mouse embryonic kidneys without the need of marker selection (FIG. 3D). It was found that after dissociating and plating Six2^{GCE} whole embryonic kidneys cells onto laminin-coated, but not gelatin or matrigel-coated plates, some cells displayed less adherence to the culture dishes which led to their detachment after 4 day culture. These floating aggregates were all GFP+ cells, suggesting differential adherence allowed selective aggregation of primary NPCs. Using the ICR strain as an example, and like in the case of Six2^{GCE}, after 4 days, formation of floating aggregates was observed that after expansion led to successful srNPC derivation. ICR-srNPCs homogenously expressed NPC markers SIX2 and SALL1, indicating the high selectivity of the CDP method. Similarly to the Six2-GFP srNPC lines, upon spinal cord co-culture, ICR-srNPCs also were able to generate renal tubular structures, suggesting their intact nephrogenic potential.

CD2AP is an important interacting protein for NEPHRIN and PODOCIN and is necessary for proper glomerulus functions and mutations in CD2AP are associated with sporadic nephrotic syndrome and focal segmental glomerulosclerosis (FSGS). Cd2ap knockout mice die within 6 weeks after birth due to compromised kidney functions. Interestingly, homozygotes of a transgenic mouse strain (Tg(tetO-GFP,-lacZ)G3Rsp, referred to as tetO for short) with transgene insertion proximal to the 5′-end of Cd2ap gene locus also die within 6 weeks. It was hypothesized that the transgenic insertion may impair the transcription of endogenous Cd2ap, thus mimicking the phenotypes of Cd2ap knockout mice. To prove this, three srNPC lines from the tetO transgenic strain were derived. Genotyping indicated that #2 is wild type while #1 and #3 are both homozygotes for the transgene. Nephron organoids were generated from the derived srNPC lines. Consistent with the hypothesis, Cd2ap was expressed in wild type (#2) while barely detected in gene-disrupted (#1 and #3) organoids (FIG. 3E). Similar to what have been reported for Cd2ap deficient mice, normal glomerulus formation was observed in nephron organoids derived from the Cd2ap gene-disrupted srNPCs. As CD2AP is critical for podocyte function, it was expected that disruption of Cd2ap would render podocyte more sensitive to LPS-induced injury. Indeed, after nephron organoids were treated with a low dosage of LPS for two days, immunostaining with antibody against active Caspase 3 clearly indicated that Cd2ap-deficient srNPC-derived glomerli accumulated more apoptotic cells than the wild type. These observations constitute a first proof of concept of the application of efficient srNPC derivation followed by fast organoid formation from genetically modified mouse strains for studying kidney organogenesis and modeling diseases.

Cells with unlimited proliferative potential are technically advantageous for gene editing. Nephrin (Nphs1) loss-of-function mutations lead to congenital nephrotic syndromes (CNS) in humans, and knockout of Nephrin in mice phenocopies the disease features of neonatal death with proteinuria. To test if srNPCs are amenable to genetic modifications the CRISPR-Cas9 system was used to knockout Nephrin in srNPCs. Stable srNPC clones were obtained after successful delivery of gene targeting constructs and drug selection. The Nephrin-KO srNPCs were used subsequently to generate organoids. In contrast to control srNPCs where a number of glomeruli with Nephrin expression were observed, gene-targeted glomeruli cells in derived organoids displayed no Nephrin expression. Together, these results highlight the possibilities that the combination of gene-editing technologies and efficient srNPC nephron organoids generation may offer for in vitro modeling of genetic kidney diseases.

### Example 5: In Vivo Developmental Potential

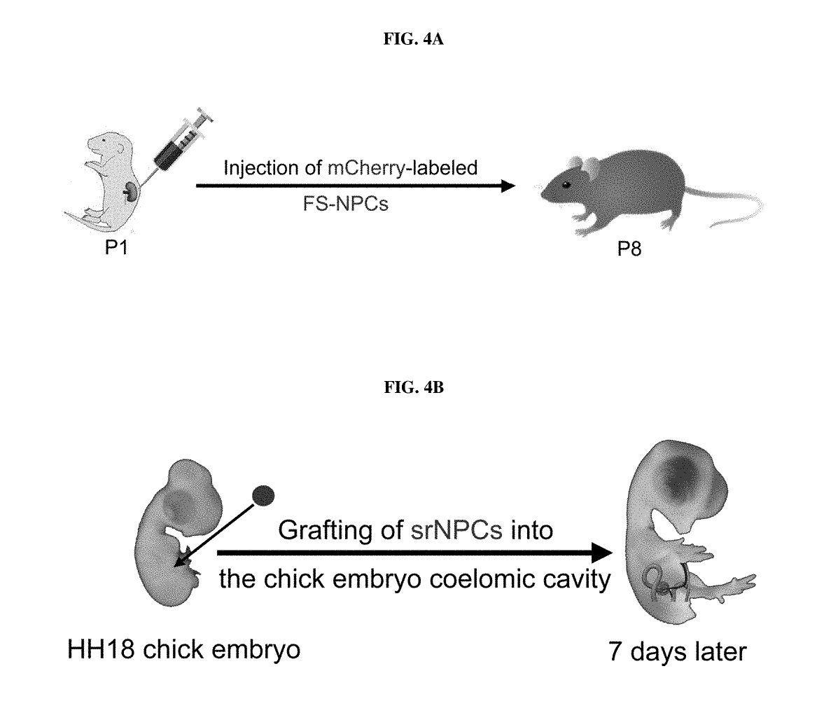

As an additional functional test, authentic srNPCs are expected to be incorporated into kidney organogenesis in vivo. Technically speaking, in utero delivery of srNPCs to their in vivo niche in mouse embryonic kidneys is extremely challenging. Since the nephrogenic niches may still persist a few days after birth, it was tested whether srNPCs could contribute to new nephron formation in neonatal kidneys. srNPCs were transplanted under the subcapsule of neonatal P0-P1 kidneys. However, contribution of srNPCs was not detected in this context. This may have been the result of failure of exposing srNPCs to the proper niche signals normally provided by UB tips within the embryonic nephrogenic zone. Nephrogenic zones in neonatal kidney are occupied by endogenous SIX2+ cells which may have prevented transplanted srNPC from accessing the UB tips. Alternatively, it could be that an aged niche (P0-P1) lacks proper inductive signals for young NPCs (E11.5-E13.5) or srNPCs to commit to nephrogenesis. In contrast to srNPCs, nephrogenesis may be already underway in FS-NPCs and therefore they might be relieved of niche signal dependency for differentiation in vivo. To test this, FS-NPCs were transplanted inside the neonatal kidney cortex at P0-P1 stages (FIG. 4A). Seven days after transplantation the mCherry-labeled FS-NPCs were observed to generate numerous tubular structures within the kidney and immunofluorescence analysis indicated that transplanted FS-NPCs generated entire LTL+ proximal tubular structures and also engrafted and formed chimeric proximal tubules and vascularized chimeric glomeruli with host cells. These data demonstrate that srNPC-derived FS-NPCs can generate all segments of nephrons in neonatal kidneys.

To independently test whether srNPCs were able to generate nephrons in vivo if exposed to signals present earlier in development, the developing chick embryo, a well-established and more accessible system for testing progenitor developmental potentials, was utilized. Small clumps of mCherry-labeled srNPC aggregates were grafted into the lateral plate mesoderm of stage HH18 chick embryos (FIG. 4B). Seven days after transplantation, the chick embryo was dissected and an intact tubular structure labeled by mCherry+ signal was found. Whole-mount staining further indicated that the mCherry-labeled srNPC aggregate gave rise to tubular structures in vivo which contained PODOXL+/WT1+ glomerulus, LTL+ proximal tubules and CDH1+ distal tubules. Importantly, these structures were spatially organized in a pattern mimicking an in vivo generated nephron (glomerulus, to proximal and to distal tubules). These results strongly support the in vivo nephrogenic potential of long-term cultured srNPCs.

### Example 6: srNPCs Generates Mini-Kidneys Producing Urine and Improve Kidney Function in an Acute Kidney Injury Model

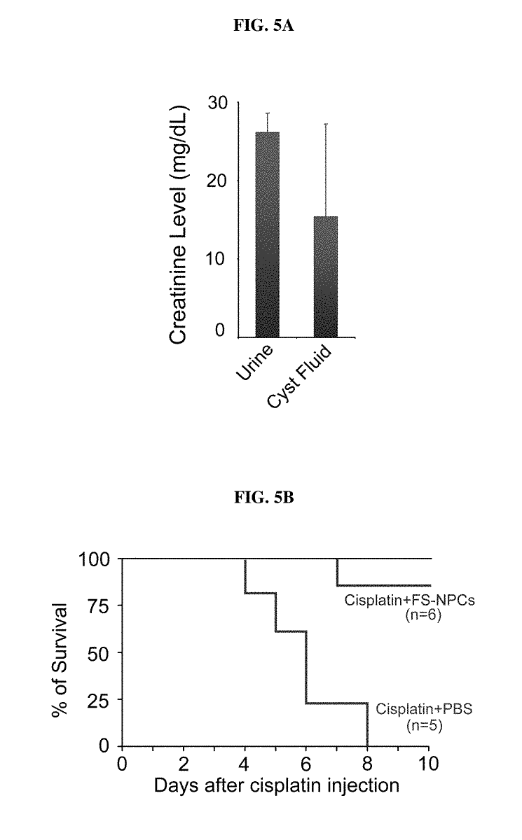

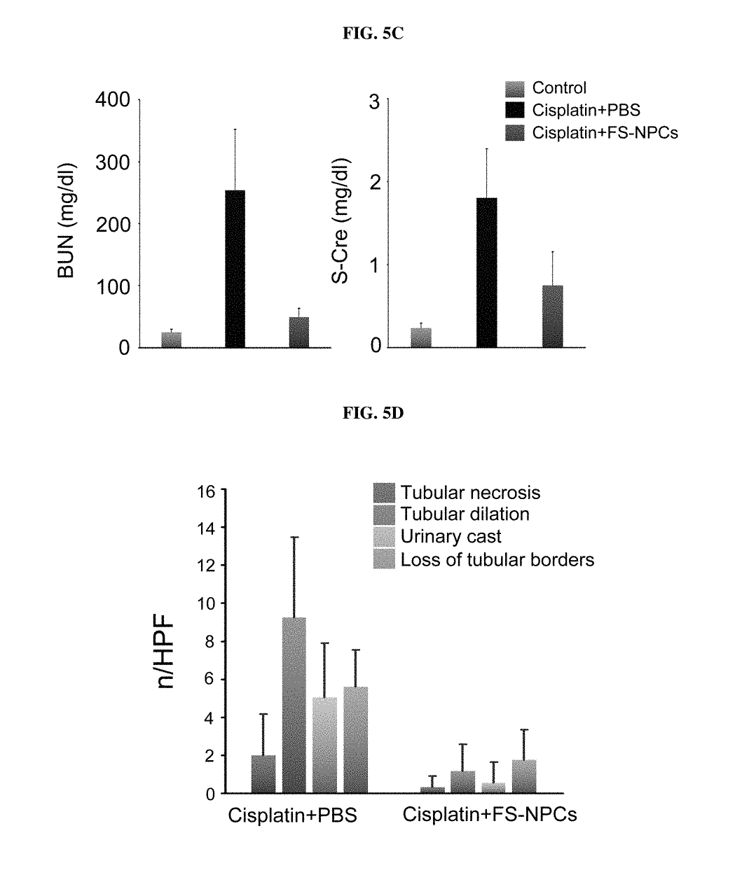

The robust in vivo nephrogenic potential of srNPCs allowed us to test whether a functional ectopic mini-kidney with urine excretion capability could be generated. mCherry-labeled srNPCs were transplanted together with spinal cord into the omentum of immunodeficient NSG mice. Two weeks later, we observed the formation of numerous cysts filled with fluid surrounded by mCherry+ cells. Host blood vessels were observed to infiltrate the mCherry+ structures Immunofluorescence analysis not only confirmed the lining of cysts by mCherry+ cells, but also revealed that srNPC could give rise to numerous LTL+ proximal tubules and DBA+ distal tubules as well as PODXL+ and WT1+ glomerulus structures. Importantly, it was observed that PODXL+ glomeruli were connected to LTL+ proximal tubules and many CD31+/mCherry− endothelial cells integrated into the WT1+/mCherry+ glomerulus, suggesting that blood vessels from the host tissue connected to the srNPC-derived glomeruli. To investigate whether the cyst fluid originated from the host circulatory system, fluorescence-conjugated low molecular weight Dextran was injected through tail vein. Two hours after injection, clear Dextran accumulation was detected in the cyst. Typical urine metabolites such as creatinine was also found in the cyst fluid, further confirming that the srNPCs can generate mini-kidneys with urine-like filtration ability in vivo (FIG. 5A).

It has been demonstrated that embryonic NPCs can improve kidney functions upon acute kidney injury. Despite their potential, and due to their scarcity, it will be difficult to use primary human fetal NPCs in clinical applications. It was determined whether srNPCs could be harnessed for restoring kidney function upon acute kidney injury. Consistent with the finding that adult kidney environment is not permissible for initiating nephron neogenesis, undifferentiated srNPCs were not able to differentiate upon transplantation into the subcapsule of adult NSG mice. Nephrogenic competent FS-NPCs. FS-NPCs were transplanted into renal subcapsule following cisplatin treatment. It was observed that mice receiving FS-NPC transplants survived longer compared to control mice (FIG. 5B). All 5 mice in the control group receiving cisplatin died within 8 days. In contrast, only 1 out of 6 mice died in the experimental group after FS-NPC transplantation. Importantly, all surviving mice showed improved renal function as measured by several biochemical parameters including significantly reduced blood urea nitrogen (BUN) and serum creatinine (S-Cre) levels (FIG. 5C). Histological analyses further revealed significant lowered levels of tubular dilation and necrosis, reduced numbers of urinary cast, and reduced occurrence of loss of tubular borders in the mice receiving FS-NPC compared to control mice receiving PBS (FIG. 5D). Moreover, transplanted FS-NPCs properly differentiated into nephron structures surrounded by CD31+ host endothelial cells. Taken together, these results show that, contrary to srNPCs, niche-independent FS-NPCs can undergo successful engraftment to adult kidney and provide a first proof-of-concept for the utilization of NPCs towards restoring kidney function upon acute injury.

### Example 7: Derivation and Long-Term Culture of Functional Human srNPCs

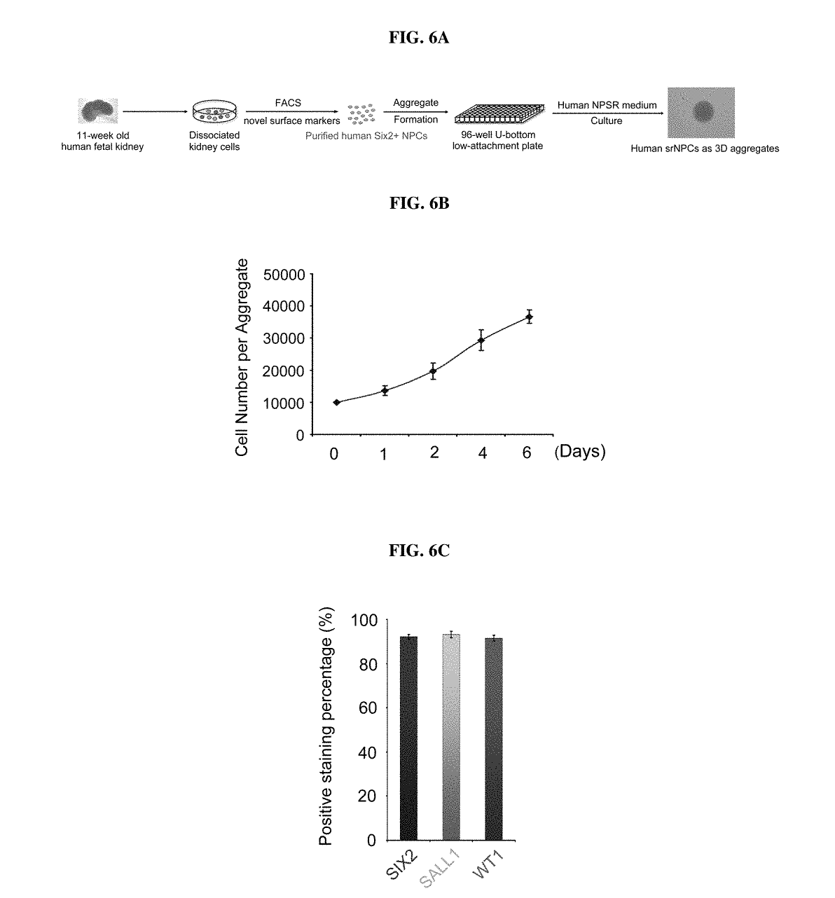

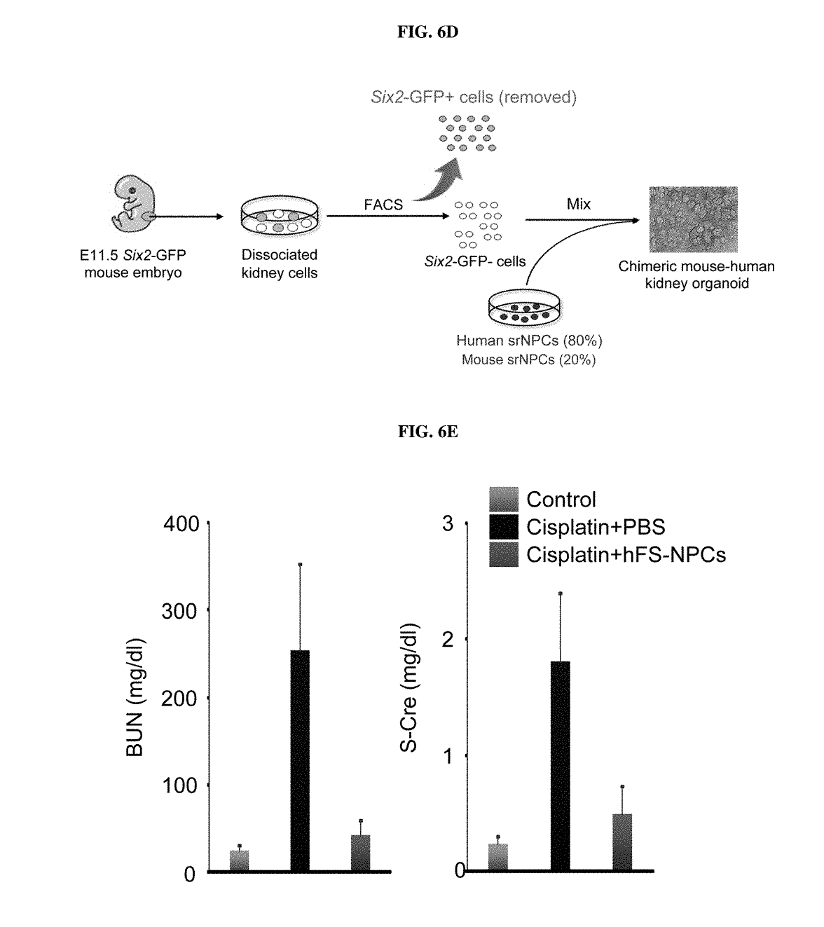

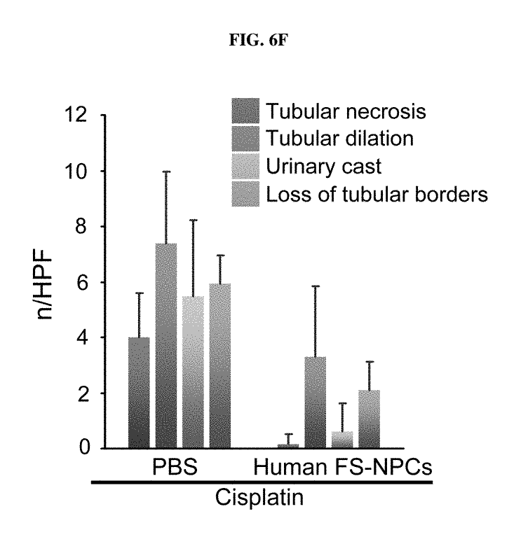

Human nephrogenesis starts around 5 weeks and ends by 36 weeks of gestation age. To investigate whether a similar strategy could be applied for the derivation of human srNPCs, aggregates were generated using unfractionated human fetal kidney cells from 11 weeks of gestation and cultured in human NPSR medium (human LIF was used instead of mouse LIF). After one week, however, all cultured human SIX2+ population was lost and the aggregates showed massive tubulogenesis. The complex microenvironment from the mixed human kidney cell populations in the aggregates may have interfered with human NPC self-renewal. To selectively enrich human NPC population, the mouse CDP method was tested. Human NPCs did not give rise to aggregates after being plated onto laminin-coated dishes, suggesting human NPCs have distinct adherent properties from mouse NPCs. Surface markers were selected that were highly enriched in primary mouse NPCs based on the RNA-seq analysis disclosed herein and combinatory testing of these surface markers was performed towards purifying human NPC fractions. It was found that EpCAM can selectively exclude a large proportion of SIX2− populations while NGFR specifically selects for SIX2+ cells. A combination of EpCAM−/NGFR+ could significantly enrich the Six2+ population from the primary human fetal kidney cells. Purified human cells could expand as 3D aggregates in human NPSR medium (FIG. 6A). Although it was observed that human srNPCs grow more slowly than mouse srNPCs (FIG. 6B), they could self-renew long term in NPSR culture with homogeneous expression of NPC protein markers including SIX2, SALL1, and WT1 (FIG. 6C), highlighting the purity of the expanded human srNPCs.

To test the functionality of long term cultured human srNPCs, an ex vivo complementary reaggregation assay was devised where human srNPCs were aggregated with mouse primary UB cells. However, nephrogeneisis was not observed in aggregated human srNPCs. This failure to undergo nephrogenesis suggests that the key molecules in mice involved in MM-UB interaction may not cross-react with those in humans. Intriguingly, however, a mixture was found containing 20% mouse srNPCs and 80% human srNPCs in the complementary reaggregation assay helped human srNPCs to generate glomerulus-like structures and allowed for successful formation of mouse-human chimeric glomeruli and distal tubules (FIG. 6D). These results suggest that signals from mouse srNPCs, or their derivatives, are effective in triggering human srNPCs to differentiate in this inter-species context. To investigate the therapeutic value of human srNPCs, human FS-NPCs were generated by exposing human srNPCs to CHIR and FGF treatment. Human FS-NPCs were transplanted into the renal subcapsule of cisplatin-treated mouse kidney. Similar to mouse srNPCs, a clear improvement in renal functions was observed as evidenced by significant decrease of BUN and serum creatinine levels (S-Cre) (FIG. 6E). Moreover, histological analyses revealed that all the pathological features underlying cisplatin nephrotoxicity such as tubular dilation and necrosis, presence of urinary cast, and loss of tubular borders were significantly attenuated in mice receiving human FS-NPCs transplants (FIG. 6F). These results demonstrate the therapeutic value of human srNPCs for clinical applications.

### Example 8: Experimental Procedures

Mouse and Human srNPC Derivation and Culture

Six2^{GCE} mice were purchased from The Jackson Laboratory. Mouse embryonic kidneys and neonatal kidneys were manually dissected and dissociated to single cells. Six2-GFP+ cells were purified by FACS sorting. 5,000 sorted GFP+ cells were seeded into one well of 96-well U-bottom low attachment plate in mouse NPSR medium containing BMP7, FGF2, Heparin, Y27632, mouse LIF, and CHIR99021. Medium was changed every two days. For passaging, srNPC aggregates were dissociated into single cells and passaged at ratios of 1:20 to 1:40 when they reached a diameter of around 1 mm (typically 4 to 6 days). For mouse strains other than Six2^{GCE}, culture-dependent purification (CDP) method was used to purify and derive srNPC lines. In brief, E12.5 mouse embryonic kidneys were dissociated into single cells and 5,000 to 10,000 cells are plated into one well of Laminin-coated 12-well plate in NPSR medium. Additional fresh medium was added after 2 days without removing the old medium. 4 to 6 days after plating, round floating aggregates were collected, dissociated and seeded into 96-well U-bottom low attachment plate in NPSR medium. Human NPCs were purified by FACS using Fluor-conjugated cell surface antibodies against human EpCAM and NGFR. 10,000 cells were seeded into one well of 96-well U-bottom low attachment plate in human NPSR medium containing BMP7, FGF2, Heparin, Y27632, human LIF, and CHIR99021. Medium was changed every two days and the aggregates were dissociated and split at 1:3 every 5 to 7 days. Information for antibodies can be found in Table 3.

### Mouse Nephron Organoid Generation and Chemical Treatment

srNPC aggregates were placed onto transwell inserts at the air-liquid interface with culture medium at the bottom containing high concentration of CHIR99021 and FGF2 for the first 2 days to generate fate-specified NPCs (FS-NPCs). The culture medium was then switched to basal medium containing 5% knockout serum replacement (KSR) for another 5 days to generate nephron organoids. DAPT at final concentration of 5 μM was used to specifically block glomerulus and proximal tubule differentiation from day 2 to day? (FS-NPCs to nephron organoids period). LPS was used at final concentration of 20 μg/ml to treat nephron organoids for 2 days to induce glomerulus damage. Low concentration of LPS (10 μg/ml) was used to treat tetO srNPC lines for two days before staining to examine cell apoptosis.

Lentiviral Labeling of mCherry and CRISPR-Cas9 Mediated Nphs1 Gene Knockout in srNPCs

CMV-driven mCherry expressing lentivirus was generated as described previously. 0.5 μg/ml of Puromycin was used to select for mCherry-expressing srNPCs after 3 days of infection. srNPCs with homogeneous expression of mCherry were obtained after 2 passages. For the lentiviral based CRISPR vector, the Cas9 vector used was modified from the lentiCRISPR vector (Addgene, plasmid #49535) with the replacement of original EFS to CMV promoter in order to enhance the expression of Cas9 in target cells, and cloning individual gRNAs into the vector. The gRNA sequence targeting mouse Nphs1 is gatggaaaaatggagagcct (SEQ ID NO: 1). The production, purification and titration of lenti-CRISPR virus are described in Liao et al., 2015 (Liao, H.-K., Gu, Y., Diaz, A., Marlett, J., Takahashi, Y., Li, M., Suzuki, K., Xu, R., Hishida, T., Chang, C.-J., et al. (2015). Use of the CRISPR/Cas9 system as an intracellular defense against HIV-1 infection in human cells. Nature Communications 6, 6413). 0.5 μg/ml of Puromycin was used to select for CRISPR-Cas9 integrated srNPCs.

### Spinal Cord Induction Assay

E11.5˜E13.5 mouse spinal cords were manually dissected and cut into small pieces. srNPC aggregates were placed in direct contact with the dorsal side of the spinal cords on air-liquid interface of transwell inserts. Culture medium containing 10% FBS was added to the bottom chamber. To test the optimal timing for isolation of FS-NPCs, srNPCs were dissected and separated from the spinal cord between day 1 to day 4 and left on air-liquid interface for spontaneous differentiation with culture medium containing 10% FBS added to the bottom chamber.

### Complementary Reaggregation Assay

E11.5 mouse embryonic kidneys from F1 embryos crossed between heterozygotes Six2^{GCE} mouse strain and wildtype B6 mice were dissected and Six2^{GCE} heterozygous kidneys were picked up under epifluorescence microscope based on GFP expression in the metanephric mesenchyme region. The Six2^{GCE} heterozygous kidneys were dissociated to single cells and sorted by FACS to collect Six2-GFP− populations. Mouse mCherry-labeled srNPCs were dissociated to single cells and reaggregated with Six2-GFP− cells at the ratio of 1:4 to 1:5, mimicking the ratio of Six2+:Six2− ratio in vivo. The reaggregates were formed overnight in 96-well U-bottom low attachment plate in culture medium containing 10% FBS and 10 μM Y27632. The next day, reaggregates were transferred to air-liquid interface with culture medium containing 10% FBS for ex vivo nephrogenesis. For human-mouse chimeric complementary reaggregation assay, 80% human srNPCs and 20% mouse srNPCs were mixed together with Six2-GFP− cells to form aggregates.

### Chick Embryo Grafting

Small sized mouse mCherry-labeled srNPC aggregates (diameter around 300 μm) were carefully placed into the lateral plate mesoderm of stage HH18 chick embryos. 7 days after grafting, the chick embryos were analyzed and mCherry+ tubular structures were identified and dissected under an epifluorescence microscope. Whole mount immunostaining was performed to characterize the identity of the tubular structures formed in vivo.

### Omentum Transplantation

2-day srNPCs and spinal cord co-culture was transplanted to the omentum of NSG mice (male and female, 8 to 12 weeks old) anesthetized by Isoflurane as reported in Yokoo et al., 2006 (Yokoo, T., Fukui, A., Ohashi, T., Miyazaki, Y., Utsunomiya, Y., Kawamura, T., Hosoya, T., Okabe, M., and Kobayashi, E. (2006). Xenobiotic kidney organogenesis from human mesenchymal stem cells using a growing rodent embryo. Jasn 17, 1026-1034). After 2-3 weeks mice were sacrificed and transplants were isolated and analyzed. 200 uL of 5 mg/ml Lucifer Yellow-labeled Dextran (10,000 MW) was injected as an intravenous bolus 2 hr before the mice were sacrificed and examined for presence of Lucifer Yellow-labeled Dextran in the cyst fluid as described in Hackl et al., 2013. Both cyst fluid and urine were collected and assayed for Creatinine levels in the same way as blood serum.

### Neonatal Renal Cortex Transplantation

For neonatal mice, newborn C57BL/6 mice (P0.5) were placed on an ice pack for up to 15 minutes to induce hypothermic anesthesia and received a 5-10 mm paramedian incision for exposure of the kidney. 3 aggregates (approximately 1.0×10⁵ cells/aggregate) of srNPCs or FS-NPCs were injected into the renal cortex area of neonates using a 29-gauge Hamilton syringe. 7 days later, the mice were sacrificed and the kidney tissues were collected for analysis.

### Cisplatin-Induced Acute Kidney Injury Mouse Model

NOD/SCID IL-2R^{null} (NSG) mice (male and female, 8 to 12 weeks old) received a subcutaneous injection of 20.0 mg/Kg cisplatin (Tocris Bioscience, Ellisville, Mo.). After 24 h, mice were transplanted with mouse or human FS-NPCs (20 aggregates, about 2 million cells), or PBS (Control) into left side renal subcapsule. Kidney tissues and blood serum samples were collected 4 days after transplantation. Mice receiving FS-NPCs transplantation survived longer and the kidney tissue and blood serum samples were collected 10 days after transplantation. Blood serum was assayed for blood urea nitrogen (BUN) and serum creatinine (S-Cre) levels using commercially available assay kits (QuantiChrom Urea Assay Kit and QuaintChrom Creatinine Assay Kit; BioAssay Systems, Hayward, Calif.) as renal function parameters. For renal subcapsule transplantation, the left kidney of NSG mice (male and female, 8 to 12 weeks old) anesthetized by Isoflurane was lifted and a pocket was created under the subcapsule using a 24-gauge catheter after puncture of needle. FS-NPC aggregates or PBS were then delivered into the pocket of renal subcapsule through the 24-gauge catheter. Collected kidney samples were fixed in 4% Paraformaldehyde (PFA), quickly frozen in ethanol, and embedded in OCT compound after PBS wash. Cryostat sections (10 μm) were stained with hematoxylin and eosin (H&E) and periodic acid-Schiff's reagent (PAS). Tubular necrosis, urinary casts, tubular dilation, and tubular borders were assessed in non-overlapping fields (high power field, HPF) as described in Imberti et al., 2015 (Imberti, B., Tomasoni, S., Ciampi, O., Pezzotta, A., Derosas, M., Xinaris, C., Rizzo, P., Papadimou, E., Novelli, R., Benigni, A., et al. (2015). Renal progenitors derived from human iPSCs engraft and restore function in a mouse model of acute kidney injury. Scientific Reports 5, 8826).

Whole Mount Immunostaining, RNA Purification, qRT-PCR and Immunoblotting

Whole mount immunostaining was performed as described in Xia et al., 2013 (Xia, Y., Nivet, E., Sancho-Martinez, I., Gallegos, T., Suzuki, K., Okamura, D., Wu, M.-Z., Dubova, I., Esteban, C. R., Montserrat, N., et al. (2013). Directed differentiation of human pluripotent cells to ureteric bud kidney progenitor-like cells. Nature Cell Biology 15, 1507-1515). RNA purification, qRT-PCR and immunoblotting were performed as described in Li et al., 2012 (Li, Z., Fei, T., Zhang, J., Zhu, G., Wang, L., Lu, D., Chi, X., Teng, Y., Hou, N., Yang, X., et al. (2012). BMP4 Signaling Acts via dual-specificity phosphatase 9 to control ERK activity in mouse embryonic stem cells. Cell Stem Cell 10, 171-182) and Wu et al., 2015 (Wu, J., Okamura, D., Li, M., Suzuki, K., Luo, C., Ma, L., He, Y., Li, Z., Benner, C., Tamura, I., et al. (2015). An alternative pluripotent state confers interspecies chimaeric competency. Nature 521, 316-321). qRT-PCR primers are listed in Table 2 and antibody information can be found in Table 3.

### RNA-seq and Principal Component Analysis

Primary Six2-GFP+ cells from E11.5, E12.5, E13.5, E16.5 and P1 were isolated from Six2^{GCE} mice and srNPCs from E11.5, E13.5, E16.5 and P1 (P20) were used for RNA-seq. To generate rpkm values from raw data, single-end 50 bp reads were mapped to the UCSC mouse transcriptome (mm9) by STAR (STAR-STAR_2.4.0f1, --outSAMstrandField intronMotif --outFilterMultimapNmax 1 --runThreadN 5), allowing for up to 10 mismatches (which is the default by STAR). Only the reads aligned uniquely to one genomic location were retained for subsequent analysis. Expression levels of all genes were estimated by Cufflink (cufflinks v2.2.1,-p 6 -G $gtf_file --max-bundle-frags 1000000000) using only the reads with exact matches. The gene expression levels of the NPC-signature genes (Table 1) were firstly transformed as logarithm scales. The program “prcomp”, a built-in program for principal component analysis in R packages, was then employed with default parameters. The variance percentage of each principal component was evaluated and the top 3 components found accounted for 84.1% of the total variance, where PC1 accounted for 46.42%, PC2 23.87% and PC3 13.81%. Those three PCs are therefore selected as candidate principal components in the further analysis. Another program “scatterplot3d” in the R packages was used to plot the 3D view of PCA, and “ggplot2” was used in 2D view of PCA.

### Example 9: In Vitro Drug Toxicity Testing

Drug candidates for treatment of cancer are tested for toxicity in vitro using nephron organoids prepared in accordance with methods herein. Nephron organoids are cultured in media comprising CHIR99021 and FGF2. Candidate drugs are then added to the culture media at concentrations ranging from 0.01× to 100×, where x is the effective concentration of the candidate drug in cultured cells. Nephron organoids are observed by microscopy and culture media is sampled at 1 hour, 4 hours, 8 hours, 24 hours, 36 hours, 48 hours, and daily thereafter for signs and markers of cell death or necrosis.

Alternatively, an individual patient is pre-screened for kidney toxicity response to a drug to treat cancer. Nephron organoids are prepared using cells from the patient using methods herein. Patient-specific nephron organoids are cultured in media comprising CHIR99021 and FGF2. Candidate drugs are then added to the culture media at concentrations ranging from 0.01× to 100×, where x is the effective concentration of the candidate drug in cultured cells. Nephron organoids are observed by microscopy and culture media is sampled at 1 hour, 4 hours, 8 hours, 24 hours, 36 hours, 48 hours, and daily thereafter for signs and markers of cell death or necrosis.

TABLE 1
Differentially Expressed NPC Genes

| Gene | Pri.E12.5 | Pri. | Pri. | Pri. | Pri. | Pri. | Pri. | Cul. | Cul. | Cul. | Cul. | |
|---|---|---|---|---|---|---|---|---|---|---|---|---|
| Symbol | Neg | E11.5 | E12.5r1 | E12.5r2 | E13.5 | E16.5 | P1 | E11.5 | E13.5 | E16.5 | P1 | LFC |
| Scarna17 | 586.94 | 0 | 0 | 0 | 0 | 0 | 0 | 0 | 0 | 595.64 | 0 | −9.20 |
| Snord91a | 507.05 | 0 | 0 | 0 | 0 | 0 | 349.12 | 0 | 0 | 0 | 0 | −8.99 |
| Mir6988 | 292.1 | 0 | 0 | 0 | 0 | 0 | 0 | 0 | 0 | 0 | 0 | −8.20 |
| Scarna2 | 221.89 | 0 | 0 | 0 | 0 | 0 | 0 | 0 | 0 | 0 | 0 | −7.80 |
| Mir7090 | 195.65 | 0 | 0 | 0 | 0 | 0 | 0 | 0 | 0 | 0 | 0 | −7.62 |
| Mir6907 | 158.6 | 0 | 0 | 0 | 0 | 0 | 0 | 128.84 | 0 | 0 | 0 | −7.32 |
| Mir99a | 139.12 | 0 | 0 | 0 | 0 | 0 | 0 | 0 | 0 | 0 | 0 | −7.13 |
| Mir6904 | 113.63 | 0 | 0 | 0 | 0 | 0 | 0 | 184.62 | 326.27 | 0 | 0 | −6.84 |
| Mir7026 | 113.63 | 0 | 0 | 0 | 322.01 | 0 | 0 | 0 | 326.27 | 0 | 0 | −6.84 |
| Hba-a1 | 472.34 | 0 | 6.03 | 2.02 | 9.37 | 278.41 | 48.11 | 0 | 0 | 0 | 0.7 | −6.56 |
| Mir199a-1 | 86.34 | 0 | 0 | 0 | 0 | 0 | 0 | 0 | 0 | 0 | 0 | −6.45 |
| Hbb-y | 3844.95 | 0 | 53.37 | 35.1 | 25.25 | 0 | 0 | 0 | 0 | 0 | 0 | −6.41 |
| Hba-x | 1559.19 | 0 | 27.55 | 15.24 | 4.12 | 0 | 0 | 0 | 0 | 0.21 | 0 | −6.12 |
| Mir7068 | 62.46 | 0 | 0 | 0 | 0 | 0 | 0 | 0 | 0 | 190.14 | 0 | −5.99 |
| Hbb-b1 | 76.31 | 0.17 | 0.63 | 0.65 | 9.08 | 411.2 | 108.2 | 0 | 0 | 0.19 | 0.63 | −5.56 |
| Mir212 | 43.89 | 0 | 0 | 0 | 0 | 0 | 0 | 0 | 0 | 0 | 0 | −5.49 |
| Mir7091 | 43.86 | 0 | 0 | 0 | 0 | 0 | 0 | 71.27 | 125.95 | 0 | 0 | −5.49 |
| Mir24-2 | 42.92 | 0 | 0 | 0 | 0 | 0 | 44.33 | 0 | 30.81 | 0 | 0 | −5.46 |
| Hbb-bh1 | 310.65 | 0 | 7.05 | 5.32 | 1.34 | 0 | 0 | 0 | 0 | 0 | 0 | −5.44 |
| Hbb-bt | 126.02 | 0 | 3.15 | 0.98 | 4.37 | 123.79 | 28.95 | 0 | 0 | 0 | 0 | −5.37 |
| Mir6981 | 39.7 | 0 | 0 | 0 | 0 | 0 | 0 | 0 | 0 | 0 | 0 | −5.35 |
| Mir450b | 36.24 | 0 | 0 | 0 | 0 | 0 | 0 | 0 | 0 | 0 | 0 | −5.22 |
| Mir6239 | 34.88 | 0 | 0 | 0 | 0 | 0 | 0 | 0 | 0 | 0 | 0 | −5.17 |
| Mir196b | 30.34 | 0 | 0 | 0 | 171.96 | 0 | 83.56 | 49.3 | 87.12 | 184.73 | 150.74 | −4.97 |
| Mir26b | 30.34 | 0 | 0 | 0 | 0 | 0 | 0 | 0 | 0 | 0 | 0 | −4.97 |
| Mir425 | 30.34 | 0 | 0 | 0 | 0 | 0 | 41.78 | 0 | 0 | 0 | 0 | −4.97 |
| Mir3061 | 21.95 | 0 | 0 | 0 | 62.19 | 0 | 0 | 35.66 | 0 | 0 | 0 | −4.52 |
| Rprm | 159.1 | 0.73 | 10.07 | 1.92 | 4.14 | 9.64 | 5.74 | 0.04 | 0.19 | 0 | 0.33 | −4.52 |
| Calb1 | 155.67 | 0.43 | 10.85 | 1.76 | 1.52 | 13.79 | 147.51 | 0.02 | 0 | 0 | 0 | −4.42 |
| S100a8 | 22.26 | 0 | 0.32 | 0 | 0 | 4.34 | 61.3 | 0.24 | 0 | 0 | 0 | −4.33 |
| Cldn3 | 54.8 | 0.51 | 3.75 | 0.18 | 3.74 | 11.23 | 12.15 | 0.17 | 0.3 | 0.71 | 0.77 | −4.23 |
| Crlf1 | 64.04 | 0.43 | 4.26 | 0.73 | 1.45 | 3.61 | 3.4 | 1.08 | 1.36 | 1.97 | 2.45 | −4.22 |
| Cldn4 | 66.29 | 0.38 | 4.69 | 0.55 | 5.65 | 70.01 | 36.61 | 0.3 | 0.44 | 1.92 | 2.62 | −4.22 |
| Cldn7 | 72.95 | 1.47 | 5.39 | 1.58 | 7.14 | 54.17 | 27.58 | 0.74 | 2.7 | 2.17 | 1.21 | −4.04 |
| Mir135a-1 | 15.16 | 0 | 0 | 0 | 0 | 0 | 0 | 0 | 0 | 0 | 0 | −4.01 |
| Wnt11 | 53.46 | 0.51 | 3.71 | 1.15 | 1.75 | 13.09 | 8.13 | 0.74 | 0.36 | 0.42 | 0.23 | −3.99 |
| Wfdc15b | 32.71 | 0.45 | 1.76 | 0.57 | 1.61 | 37.19 | 166.49 | 0.13 | 0 | 0 | 0 | −3.96 |
| Cldn6 | 120.54 | 3.24 | 11.24 | 2.45 | 10.43 | 39.52 | 17.35 | 0.51 | 0.91 | 0.96 | 0.42 | −3.95 |
| Tbx2 | 61.54 | 1.17 | 5.66 | 1.26 | 3.77 | 26.11 | 12.6 | 28.05 | 24.47 | 9.59 | 26.07 | −3.81 |
| S100a9 | 13.91 | 0.48 | 0.19 | 0 | 0 | 7.03 | 49.6 | 0 | 0 | 0 | 0 | −3.77 |
| Ret | 22.62 | 0.1 | 1.43 | 0.17 | 0.35 | 1.62 | 1.27 | 0.09 | 0.14 | 0.08 | 0.42 | −3.71 |
| Mir3102 | 12.11 | 0 | 0 | 0 | 0 | 0 | 0 | 19.68 | 0 | 0 | 0 | −3.71 |
| Gata3 | 26.85 | 0.03 | 1.79 | 0.49 | 0.64 | 10.12 | 5.59 | 0.12 | 1.12 | 0.08 | 0.72 | −3.70 |
| Mir27a | 189.81 | 0 | 0 | 27.42 | 0 | 0 | 74.68 | 176.23 | 0 | 412.76 | 404.18 | −3.70 |
| Mal2 | 28.67 | 0 | 2.12 | 0.56 | 0.48 | 9.6 | 18.23 | 0.02 | 0.03 | 0.13 | 0 | −3.66 |
| Mir3062 | 11.63 | 0 | 0 | 0 | 32.95 | 0 | 0 | 0 | 33.39 | 0 | 0 | −3.66 |
| Plac8 | 68.31 | 2.34 | 7.33 | 1.8 | 11.04 | 88.02 | 112.62 | 3.98 | 4 | 1.02 | 7.75 | −3.64 |
| Esrp1 | 18.6 | 0.08 | 1.01 | 0.23 | 0.52 | 5.19 | 2.34 | 0 | 0 | 0 | 0 | −3.60 |
| Pou3f3os | 26.26 | 0 | 2.27 | 0.24 | 1.59 | 9.77 | 32.42 | 0 | 0 | 0 | 0 | −3.60 |
| Mir6374 | 10.73 | 0 | 0 | 0 | 0 | 0 | 0 | 0 | 0 | 0 | 0 | −3.55 |
| Mir433 | 10.71 | 0 | 0 | 0 | 0 | 0 | 0 | 0 | 0 | 0 | 0 | −3.55 |
| Cdh1 | 18.71 | 0.02 | 1.18 | 0.21 | 1.44 | 25.67 | 34.52 | 0.02 | 0.37 | 0.57 | 0.23 | −3.54 |
| Col1a1 | 175.21 | 5.91 | 20.57 | 7.98 | 18.61 | 139.7 | 40.01 | 5.63 | 8.37 | 2.56 | 3.06 | −3.53 |
| Hic1 | 77.75 | 3.59 | 9.71 | 2.26 | 8.5 | 29.07 | 14.67 | 1.64 | 0.36 | 0.44 | 0.36 | −3.49 |
| Mir6338 | 9.92 | 0 | 0 | 0 | 0 | 0 | 0 | 0 | 0 | 0 | 0 | −3.45 |
| Emx2 | 23.72 | 0.35 | 2.37 | 0.2 | 1.91 | 3.88 | 5.45 | 0.02 | 0.66 | 2.16 | 2.16 | −3.44 |
| Cdh16 | 20.36 | 0.17 | 1.69 | 0.28 | 5.54 | 112.46 | 140.57 | 0.03 | 0 | 0.03 | 0 | −3.43 |
| Krt23 | 19.11 | 0 | 1.43 | 0.33 | 1.16 | 6.79 | 6.84 | 0 | 0.06 | 0.94 | 0.31 | −3.42 |
| Tacstd2 | 18.46 | 0.1 | 1.34 | 0.3 | 3.43 | 51.06 | 38.53 | 0.03 | 0.1 | 0.75 | 1.13 | −3.42 |
| Col3a1 | 183.32 | 5.77 | 21.18 | 11.31 | 14.81 | 163.12 | 112.01 | 23.78 | 2.36 | 1.98 | 1.24 | −3.42 |
| Kif12 | 16.14 | 0 | 1.2 | 0.05 | 3.23 | 23.9 | 13.34 | 0 | 0 | 0 | 0 | −3.40 |
| Cd248 | 57.61 | 2.6 | 7.27 | 2.33 | 4.55 | 23.51 | 6.72 | 10.73 | 14.52 | 1.24 | 2.54 | −3.34 |
| Tbx3 | 17.29 | 0.03 | 1.3 | 0.32 | 0.58 | 6.62 | 4.58 | 0 | 0.04 | 0.11 | 0 | −3.34 |
| Ap1m2 | 20.89 | 0.3 | 2.16 | 0.27 | 1.37 | 13.42 | 18.83 | 0.03 | 0.05 | 0.27 | 0.62 | −3.30 |
| Pdzk1ip1 | 10.91 | 0 | 0.29 | 0.13 | 2.12 | 209.28 | 128.99 | 0 | 0 | 0 | 0 | −3.30 |
| Mir6340 | 8.76 | 0 | 0 | 0 | 0 | 0 | 0 | 0.01 | 0 | 0 | 0 | −3.29 |
| Mir496b | 8.54 | 0 | 0 | 0 | 0 | 0 | 0 | 0 | 0 | 0 | 0 | −3.25 |
| Crb3 | 18.85 | 0.4 | 1.69 | 0.52 | 2.2 | 13.35 | 15.54 | 0 | 0.08 | 0.26 | 0.14 | −3.24 |
| 1700011H14Rik | 16.3 | 0.08 | 1.36 | 0.32 | 0.81 | 14.43 | 38.63 | 0 | 0.08 | 0 | 0 | −3.23 |
| Krt7 | 11.68 | 0.22 | 0.56 | 0.14 | 2.75 | 135.06 | 84.91 | 0 | 0.23 | 0 | 0.1 | −3.23 |
| Hnf1b | 16.65 | 0.21 | 1.61 | 0.16 | 3.32 | 15.12 | 14.74 | 0 | 0 | 0 | 0.36 | −3.23 |
| Cldn8 | 16.18 | 0 | 1.52 | 0.17 | 1.01 | 11.32 | 26.94 | 0 | 0 | 0 | 0 | −3.22 |
| Tmem59l | 35.71 | 3.05 | 3.77 | 2.14 | 1.76 | 1.1 | 0.56 | 0.1 | 0.12 | 0.2 | 0.53 | −3.21 |
| S100a6 | 31.28 | 1.79 | 4.17 | 0.81 | 2.56 | 276.79 | 120.64 | 92.39 | 104.16 | 79.18 | 46.51 | −3.21 |
| Pdgfra | 31.92 | 0.51 | 3.16 | 1.96 | 1.92 | 13.37 | 14.38 | 0.15 | 0.2 | 0.16 | 0.13 | −3.21 |
| Aldh1a3 | 13.99 | 0.12 | 1.11 | 0.14 | 0.46 | 5.81 | 4.51 | 5.21 | 4.11 | 9.35 | 8.78 | −3.21 |
| Col1a2 | 143.22 | 6.81 | 19.04 | 10.56 | 13.33 | 137.08 | 109.42 | 3.98 | 4.92 | 0.39 | 1.08 | −3.19 |
| Crabp1 | 506.67 | 26.43 | 76.74 | 32.79 | 45.44 | 25.09 | 4.98 | 52.55 | 23.15 | 19.11 | 18.44 | −3.19 |
| Vsnl1 | 11.57 | 0.09 | 0.69 | 0.08 | 0.13 | 0.09 | 0 | 0 | 0 | 0 | 0 | −3.18 |
| Rab25 | 14.88 | 0.27 | 1.08 | 0.42 | 0.91 | 22.83 | 18.47 | 0.47 | 1.19 | 1.17 | 0.32 | −3.18 |
| Krt8 | 32.59 | 2.01 | 3.73 | 1.68 | 3.49 | 81.5 | 28.78 | 0.19 | 0.34 | 0.63 | 0.85 | −3.18 |
| Pdgfrb | 32.08 | 1.37 | 3.83 | 1.51 | 2.67 | 14.79 | 14.08 | 5.35 | 3.82 | 1.62 | 1.33 | −3.17 |
| Mir6418 | 7.94 | 0 | 0 | 0 | 45 | 0 | 0 | 0 | 0 | 24.17 | 0 | −3.16 |
| Epcam | 101.45 | 7.07 | 13.04 | 7.91 | 10.41 | 50.1 | 70.57 | 14.24 | 22.08 | 53.51 | 47.66 | −3.16 |
| Snora15 | 7.66 | 0 | 0 | 0 | 0 | 0 | 0 | 0 | 0 | 0 | 0 | −3.11 |
| Thy1 | 20.24 | 1 | 1.89 | 1.05 | 3.8 | 34.51 | 16.69 | 0.2 | 0.26 | 0.05 | 0.27 | −3.10 |
| Cthrc1 | 32.62 | 0.64 | 4.22 | 1.73 | 3.4 | 8.44 | 6.88 | 3.29 | 3.28 | 8.52 | 4.82 | −3.08 |
| S1pr3 | 33.8 | 1.4 | 3.99 | 2.24 | 2.4 | 11.56 | 10.55 | 11.24 | 9.87 | 9.05 | 8.46 | −3.08 |
| Spint1 | 16.26 | 0.96 | 1.6 | 0.54 | 2.48 | 30.01 | 25.39 | 1.31 | 3.38 | 10 | 6.38 | −3.06 |
| St14 | 11.28 | 0.34 | 0.76 | 0.19 | 1 | 10.84 | 11.32 | 1.03 | 3.78 | 4.7 | 5.21 | −3.06 |
| Tfap2a | 12.14 | 0.09 | 0.95 | 0.25 | 0.2 | 0.95 | 1.63 | 0.02 | 0 | 0 | 0.05 | −3.04 |
| Stmn2 | 47.49 | 1.99 | 6.86 | 3.13 | 2.51 | 4.63 | 0.8 | 26.43 | 13.33 | 3.14 | 2.72 | −3.02 |
| Wnt6 | 10.51 | 0.37 | 0.8 | 0.09 | 0.62 | 0.72 | 0.67 | 0.07 | 0.08 | 0.13 | 0.07 | −2.99 |
| Car2 | 15.53 | 0.73 | 1.5 | 0.68 | 0.29 | 7.07 | 34.52 | 0.45 | 0.74 | 0.37 | 0.43 | −2.98 |
| Nfib | 33.12 | 0.38 | 4.05 | 2.69 | 1.34 | 4.92 | 5.25 | 4.95 | 2.19 | 0.2 | 1.36 | −2.96 |
| Tmem119 | 44.18 | 2.74 | 6.48 | 3.13 | 4.23 | 15.5 | 10.43 | 5.13 | 6 | 2 | 2.62 | −2.96 |
| Aldh1a1 | 9.49 | 0 | 0.6 | 0.11 | 0.08 | 0.37 | 0.91 | 0.02 | 0 | 0 | 0 | −2.95 |
| Alas2 | 9.5 | 0.18 | 0.5 | 0.23 | 0.36 | 4.34 | 1.49 | 0.08 | 0.45 | 0.14 | 0.24 | −2.94 |
| Arg2 | 13.67 | 0.19 | 1.38 | 0.44 | 0.83 | 4.41 | 3.87 | 0.29 | 0.52 | 0.48 | 0.56 | −2.94 |
| Rarg | 32.71 | 1.4 | 4.99 | 1.8 | 3.29 | 22.43 | 7.61 | 9.67 | 9.38 | 11.15 | 7.42 | −2.94 |
| Ldb2 | 50.81 | 1.92 | 7.97 | 3.6 | 2.44 | 20.67 | 22.39 | 0.69 | 0.41 | 0 | 0 | −2.93 |
| Sncg | 44.93 | 2.32 | 7.51 | 2.52 | 1.62 | 9.85 | 0.93 | 15.09 | 14 | 1.58 | 3.61 | −2.93 |
| Twist1 | 65.39 | 1.56 | 8.55 | 6.94 | 1.96 | 3.91 | 4.5 | 0 | 0 | 0 | 0 | −2.92 |
| Pou3f3 | 11.59 | 0.21 | 1.08 | 0.24 | 1.06 | 4.65 | 9.5 | 0 | 0 | 0 | 0 | −2.92 |
| Fev | 9.5 | 0 | 0.7 | 0.07 | 0 | 0.23 | 0 | 0 | 0 | 0 | 0 | −2.92 |
| Islr | 81.62 | 8.93 | 12.97 | 7.36 | 16.97 | 51.69 | 35.9 | 9.44 | 8.04 | 1.82 | 1.3 | −2.89 |
| Mal | 9.08 | 0 | 0.63 | 0.1 | 0.74 | 46.36 | 81.04 | 0.02 | 0 | 0.03 | 0.17 | −2.88 |
| Asb4 | 49.35 | 1.62 | 7.14 | 4.5 | 0.86 | 2.99 | 2.57 | 35.06 | 22.91 | 0.11 | 1.06 | −2.88 |
| Cxcl14 | 16.14 | 1.04 | 1.82 | 0.85 | 1.53 | 8.21 | 13.9 | 0.03 | 0.39 | 0.82 | 1.76 | −2.88 |
| Cfh | 24.34 | 0.48 | 3.28 | 1.63 | 1.36 | 20.32 | 49.23 | 0.02 | 0 | 0 | 0.03 | −2.87 |
| Bgn | 49.24 | 1.62 | 9.39 | 2.38 | 5.86 | 92.71 | 74.83 | 23.29 | 22.11 | 17.14 | 21.75 | −2.87 |
| Hsd3b6 | 6.79 | 0 | 0.13 | 0.02 | 0.05 | 1.77 | 1.32 | 0 | 0 | 0 | 0 | −2.86 |
| Pid1 | 24.2 | 0.65 | 2.95 | 2.06 | 0.82 | 4.92 | 8.69 | 0.85 | 0.16 | 0 | 0 | −2.85 |
| 2610203C20Rik | 35.32 | 1.16 | 5.89 | 2.28 | 1.93 | 5.57 | 10.05 | 8.74 | 3.84 | 0.95 | 1.09 | −2.84 |
| Wnt9b | 8.94 | 0.02 | 0.71 | 0.08 | 0.56 | 3.81 | 2.11 | 0 | 0 | 0.02 | 0.03 | −2.83 |
| Ebf3 | 27.9 | 0.48 | 4.18 | 2.04 | 1.96 | 2.48 | 0.45 | 4.68 | 3.17 | 0.71 | 2.38 | −2.81 |
| Mme | 15.77 | 0.38 | 1.72 | 1.08 | 0.77 | 1.75 | 12.13 | 0.61 | 0.31 | 0.03 | 0 | −2.80 |
| Prg2 | 5.96 | 0 | 0 | 0 | 0 | 0 | 0.12 | 0.35 | 0.25 | 0.13 | 0 | −2.80 |
| Arhgef19 | 10.02 | 0.46 | 0.82 | 0.39 | 0.73 | 4.59 | 1.95 | 1.51 | 1.53 | 2.17 | 1.45 | −2.78 |
| Gm12238 | 5.85 | 16.37 | 0 | 0 | 16.57 | 31.7 | 0 | 0 | 16.78 | 0 | 29.04 | −2.78 |
| Ecm1 | 68.81 | 6.44 | 12.57 | 6.05 | 11.97 | 80.02 | 77.54 | 6.67 | 13.52 | 20 | 13.9 | −2.76 |
| Mecom | 31.67 | 0.69 | 4.64 | 3.18 | 1.71 | 4.15 | 13.17 | 2.67 | 0.13 | 0.47 | 0.48 | −2.73 |
| Anxa2 | 378.74 | 38.3 | 76.16 | 36.77 | 57.67 | 380.6 | 175.57 | 700.12 | 818.33 | 973.43 | 703.17 | −2.72 |
| Fibin | 26.24 | 0.82 | 3.05 | 3.27 | 1.42 | 6.65 | 21.63 | 0.5 | 0.89 | 0.22 | 0.07 | −2.71 |
| Tbx18 | 16.54 | 0.38 | 1.87 | 1.49 | 0.23 | 0.6 | 0.35 | 0 | 0.04 | 0 | 0.15 | −2.71 |
| Rarres2 | 28.4 | 3.04 | 4.44 | 2.63 | 10.83 | 62.39 | 41.45 | 11 | 18.62 | 9.52 | 10.15 | −2.70 |
| Bspry | 8.74 | 0.2 | 0.7 | 0.31 | 0.81 | 8.16 | 5 | 0.03 | 0.2 | 0.16 | 0.18 | −2.69 |
| Gata2 | 19.23 | 0.66 | 2.75 | 1.51 | 1.12 | 14.09 | 4.09 | 2.52 | 4.01 | 3.98 | 6.5 | −2.69 |
| Col6a2 | 19.11 | 1.87 | 3.14 | 1.12 | 5.02 | 41.3 | 30.17 | 4.99 | 5.47 | 2.58 | 2.78 | −2.68 |
| Pdzrn3 | 31.54 | 1.71 | 5.46 | 2.74 | 2.01 | 4.42 | 5.09 | 4.3 | 1.27 | 0.51 | 0.59 | −2.67 |
| Foxd1 | 149.75 | 12.82 | 25.28 | 20.1 | 17.51 | 19.36 | 14.71 | 0.12 | 0.24 | 0.37 | 0.97 | −2.67 |
| Alx1 | 10.7 | 0.17 | 1.13 | 0.58 | 0.41 | 7.34 | 6.43 | 0 | 0 | 0.04 | 0 | −2.66 |
| Col6a1 | 19.63 | 2.12 | 2.9 | 1.68 | 4.3 | 37.75 | 40.71 | 11.08 | 13.08 | 9.05 | 7.36 | −2.65 |
| Agtr2 | 11.42 | 0.03 | 1.06 | 0.91 | 1.02 | 7.3 | 11.74 | 0 | 0 | 0 | 0 | −2.65 |
| A4galt | 10.52 | 0.93 | 1.06 | 0.63 | 1.52 | 5.83 | 3.74 | 5.68 | 3.29 | 2.34 | 3.03 | −2.64 |
| Tmem54 | 9 | 0.09 | 1.08 | 0.13 | 0.52 | 4.11 | 0.17 | 0.15 | 0.18 | 0.19 | 0.3 | −2.64 |
| Pcolce | 86.62 | 17.7 | 16.73 | 9.59 | 26.47 | 91.56 | 89.66 | 199.4 | 339.83 | 371.74 | 342.66 | −2.63 |
| Vcam1 | 14.7 | 0.36 | 2 | 1.1 | 0.63 | 5.44 | 15.35 | 0.06 | 0.42 | 0 | 0.26 | −2.62 |
| F2rl1 | 7.05 | 0.09 | 0.53 | 0.09 | 0.52 | 3.23 | 5.65 | 0.61 | 0.96 | 0.65 | 0.8 | −2.62 |
| Lpar1 | 32.7 | 1.77 | 5.28 | 3.74 | 1.99 | 5.49 | 5.9 | 3.1 | 3.18 | 1.87 | 3 | −2.61 |
| Kitl | 12.51 | 0.03 | 1.73 | 0.69 | 0.21 | 1.22 | 1.85 | 2.11 | 1.19 | 0.4 | 0.44 | −2.61 |
| Sept4 | 15.41 | 0.97 | 2.43 | 0.94 | 2.5 | 10.15 | 46.85 | 0.43 | 0.78 | 1.54 | 1.24 | −2.61 |
| Gata6 | 17.56 | 0.31 | 2.42 | 1.71 | 0.55 | 2.98 | 3.74 | 1.47 | 1.05 | 2.18 | 1.59 | −2.60 |
| Fap | 11.66 | 0.25 | 1.47 | 0.72 | 0.62 | 2.73 | 4.12 | 0.18 | 0.09 | 0 | 0 | −2.60 |
| 5930403L14Rik | 8.09 | 0.15 | 0.7 | 0.31 | 0.31 | 1.39 | 5.5 | 0 | 0 | 0 | 0 | −2.59 |
| Matn2 | 14.99 | 0.56 | 1.83 | 1.47 | 1.52 | 7.06 | 7.57 | 4 | 4.04 | 1.31 | 3.53 | −2.59 |
| Rhoj | 21.02 | 0.68 | 4.32 | 1.03 | 1.77 | 13.56 | 11.37 | 0.48 | 0.91 | 1.59 | 1.2 | −2.58 |
| Mir8119 | 4.99 | 0 | 0 | 0 | 0 | 0 | 0 | 0 | 0 | 0 | 0 | −2.58 |
| Rgs5 | 20.56 | 1.05 | 2.68 | 2.54 | 0.77 | 5.51 | 13.19 | 32.51 | 19.02 | 19.28 | 21.46 | −2.58 |
| Rxfp2 | 11.57 | 0.12 | 1.66 | 0.6 | 1.1 | 2.36 | 0.23 | 0.07 | 0 | 0 | 0 | −2.56 |
| Brinp1 | 13.68 | 0.41 | 1.69 | 1.29 | 0.59 | 1.09 | 0.93 | 0.03 | 0 | 0.13 | 0 | −2.56 |
| Sema6a | 25.14 | 1.77 | 4.25 | 2.66 | 2.47 | 3.84 | 2.53 | 9.3 | 8.15 | 1.68 | 2.61 | −2.55 |
| Car3 | 20.2 | 1.85 | 2.59 | 2.68 | 2.87 | 3.81 | 16.8 | 0 | 1.43 | 1.83 | 2.57 | −2.54 |
| Smoc2 | 10.3 | 0.18 | 1.49 | 0.39 | 4.28 | 30.79 | 7.27 | 0 | 0.18 | 0.73 | 0.41 | −2.54 |
| Mapk13 | 32.63 | 5.31 | 6.2 | 3.39 | 6.23 | 14.43 | 10.61 | 2.14 | 2.72 | 6.69 | 2.64 | −2.54 |
| Ddr2 | 10.24 | 0.12 | 1.29 | 0.62 | 0.59 | 3.59 | 3.76 | 0.09 | 0.07 | 0.01 | 0.02 | −2.52 |
| Ntn1 | 25.84 | 4.09 | 4.95 | 2.4 | 8.69 | 19.34 | 5.97 | 0.31 | 0.5 | 0.78 | 0.91 | −2.52 |
| BC100530 | 4.74 | 0 | 0 | 0 | 0 | 0.94 | 7.95 | 0 | 0 | 0 | 0 | −2.52 |
| Slc4a1 | 6.89 | 0.57 | 0.43 | 0.32 | 0.43 | 0.65 | 0.98 | 0.01 | 0 | 0.02 | 0 | −2.52 |
| Postn | 17.71 | 1.1 | 2.84 | 1.72 | 4.62 | 28.66 | 14.01 | 20.47 | 8.13 | 0.44 | 1 | −2.51 |
| Kdf1 | 8.4 | 0.55 | 0.97 | 0.34 | 0.71 | 4.75 | 4.16 | 1.25 | 4.21 | 1.74 | 2.13 | −2.51 |
| Col16a1 | 11.73 | 1.1 | 1.8 | 0.7 | 2.66 | 19.45 | 9.86 | 3.83 | 4.85 | 6.29 | 3.7 | −2.50 |
| Stfa1 | 4.6 | 0 | 0 | 0 | 0 | 1.04 | 18.32 | 0 | 0.37 | 0 | 0 | −2.49 |
| Krt18 | 78.99 | 11.1 | 14.74 | 12.16 | 14.02 | 191.66 | 69.98 | 7.67 | 17.43 | 17.93 | 17.42 | −2.47 |
| 4930412O13Rik | 6.79 | 0 | 0.72 | 0.1 | 0.2 | 0.87 | 0.84 | 0 | 0.33 | 0.11 | 0.5 | −2.47 |
| Daam2 | 12.6 | 0.96 | 1.96 | 1 | 1.76 | 4.43 | 4.76 | 1.97 | 3.04 | 1.06 | 1.77 | −2.46 |
| Col5a2 | 17.12 | 0.79 | 2.99 | 1.63 | 2.1 | 12.16 | 13.12 | 0.01 | 0.02 | 0.05 | 0.04 | −2.45 |
| Olfml3 | 62.19 | 10.27 | 14.39 | 6.78 | 15.36 | 78.67 | 35.79 | 5.09 | 7.5 | 2.61 | 3.82 | −2.45 |
| Creb3l1 | 14.87 | 1.52 | 2.44 | 1.39 | 3.01 | 15.85 | 7.54 | 5.71 | 6.66 | 4.72 | 2.83 | −2.44 |
| Ifitm3 | 42.69 | 3.28 | 9.53 | 4.74 | 10.31 | 160.26 | 254.77 | 288.38 | 261.81 | 481.54 | 376.7 | −2.43 |
| Grb7 | 41.87 | 4.34 | 8.6 | 5.41 | 5.13 | 16.27 | 12.01 | 1.08 | 1.61 | 5.29 | 8.07 | −2.42 |
| Egfl6 | 11.57 | 0.28 | 1.86 | 0.84 | 1.09 | 1.94 | 25.97 | 0 | 0 | 0.03 | 0 | −2.42 |
| Tmed6 | 7.11 | 0 | 1.04 | 0 | 0.24 | 2.82 | 11.33 | 0 | 0.12 | 0.13 | 0.41 | −2.42 |
| Sgpp2 | 7.33 | 0.61 | 0.81 | 0.35 | 0.83 | 4.27 | 7.06 | 0.99 | 1.51 | 1.21 | 1.57 | −2.40 |
| Lamc2 | 13.56 | 1.16 | 2.22 | 1.31 | 1.93 | 11.12 | 13.65 | 21.66 | 17.64 | 48.21 | 22.07 | −2.40 |
| Arhgef16 | 8.61 | 0.73 | 1.01 | 0.66 | 1.36 | 4.79 | 5.71 | 0.48 | 1.13 | 1.06 | 1.14 | −2.39 |
| Akap12 | 23.6 | 1.88 | 4.57 | 2.85 | 4.9 | 19.52 | 14.89 | 5.5 | 4.43 | 4.74 | 5.02 | −2.38 |
| Figf | 11.57 | 0.92 | 2.1 | 0.73 | 1.67 | 14.53 | 10.63 | 0.8 | 1.13 | 0.65 | 0.65 | −2.38 |
| Wdr86 | 30.07 | 6.11 | 6.2 | 3.75 | 8.54 | 19.16 | 8.87 | 11 | 11.54 | 4.75 | 3.87 | −2.38 |
| Lgals1 | 724.02 | 145.32 | 175.7 | 102.7 | 214.41 | 574.23 | 256.47 | 851.24 | 483.45 | 612.07 | 342.79 | −2.37 |
| Adamts18 | 7.34 | 0.28 | 0.89 | 0.35 | 0.18 | 0.16 | 0.22 | 0.02 | 0.02 | 0 | 0.03 | −2.36 |
| Nfia | 8.56 | 0.05 | 1.1 | 0.62 | 0.33 | 2.89 | 4.49 | 0.03 | 0.06 | 0.41 | 0.26 | −2.36 |
| Rnf43 | 5.84 | 0.06 | 0.61 | 0.09 | 0.21 | 2.14 | 1.19 | 0.02 | 0.02 | 0.06 | 0.03 | −2.34 |
| H2-DMb2 | 5.22 | 0.13 | 0.41 | 0.05 | 0.27 | 1.4 | 0.97 | 0 | 0 | 0.14 | 0.23 | −2.34 |
| Twist2 | 16.62 | 0.97 | 3.07 | 1.9 | 1.61 | 5.57 | 1.12 | 5.54 | 9.8 | 4.22 | 6.64 | −2.34 |
| Fli1 | 13.9 | 0.11 | 2.94 | 0.96 | 0.32 | 3.33 | 7.59 | 0.02 | 0 | 0 | 0 | −2.34 |
| Gria4 | 7.81 | 0.19 | 0.95 | 0.54 | 0.77 | 2.97 | 6 | 0 | 0 | 0 | 0 | −2.34 |
| Bai2 | 9.32 | 1.12 | 1.44 | 0.65 | 1.33 | 5.14 | 1.07 | 2.48 | 3.07 | 4.21 | 2.82 | −2.34 |
| Tfap2b | 4.95 | 0 | 0.34 | 0.02 | 0.45 | 1.17 | 8.13 | 0.01 | 0 | 0 | 0 | −2.33 |
| Cyp7b1 | 9.85 | 0.74 | 1.43 | 0.88 | 1.27 | 5.3 | 6.14 | 0 | 0 | 0 | 0 | −2.33 |
| Loxl1 | 18.68 | 4.21 | 3.88 | 1.95 | 5.28 | 13.09 | 24.82 | 4.6 | 5.35 | 10.81 | 9.21 | −2.33 |
| Fxyd1 | 32.03 | 8.39 | 6.15 | 5.08 | 14.22 | 43.51 | 31.78 | 31.67 | 32.52 | 7.79 | 6.04 | −2.32 |
| Tgm5 | 7.88 | 0.2 | 1.08 | 0.48 | 0.77 | 2.46 | 0 | 0 | 0 | 0 | 0 | −2.32 |
| Col5a1 | 34.86 | 5.57 | 7.79 | 4.59 | 6 | 18.34 | 10.03 | 6.44 | 5.68 | 4.82 | 4.29 | −2.32 |
| Slc16a2 | 8.71 | 0.45 | 1.28 | 0.62 | 0.83 | 4.47 | 6.31 | 3.38 | 1.09 | 0.89 | 0.62 | −2.32 |
| Arap1 | 38.89 | 5.71 | 9.35 | 4.68 | 10.5 | 37.89 | 21.97 | 5.3 | 7.03 | 7.98 | 7.1 | −2.32 |
| Gm5483 | 3.93 | 0 | 0 | 0 | 0 | 1.07 | 18.93 | 0 | 0 | 0 | 0 | −2.30 |
| Mir6386 | 36.34 | 0 | 13.18 | 0 | 0 | 0 | 0 | 0 | 34.78 | 0 | 0 | −2.30 |
| Dhrs3 | 140.92 | 18.98 | 36.98 | 18.74 | 21.22 | 95.26 | 61.38 | 1.05 | 0.41 | 0.22 | 0.18 | −2.30 |
| Bcl11a | 7.71 | 0.12 | 0.95 | 0.6 | 0.06 | 0.48 | 0.38 | 0.04 | 0 | 0.26 | 0 | −2.29 |
| Synpo | 7.64 | 0.18 | 1.06 | 0.47 | 0.37 | 2.18 | 1.78 | 2.94 | 2.33 | 3.11 | 1.87 | −2.29 |
| Tgfb1i1 | 20.62 | 1.52 | 4.53 | 2.31 | 2.86 | 19.09 | 7.27 | 2.7 | 3.91 | 2.52 | 1.84 | −2.29 |
| Gchfr | 10.38 | 1.26 | 1.82 | 0.85 | 1.1 | 20.27 | 3.73 | 1.88 | 0.19 | 0.98 | 0.64 | −2.29 |
| Ocln | 5.15 | 0.08 | 0.43 | 0.1 | 0.39 | 3.38 | 6.53 | 0.01 | 0.03 | 0.08 | 0 | −2.28 |
| Rgl1 | 29.44 | 3.42 | 6.61 | 3.96 | 4.41 | 16.45 | 12.18 | 7.73 | 8.82 | 6.41 | 7.39 | −2.28 |
| Col9a2 | 8.24 | 0.97 | 1.16 | 0.66 | 1.83 | 6.4 | 1.59 | 0.51 | 1.33 | 2.59 | 0.91 | −2.27 |
| Kcnj2 | 7.48 | 0.04 | 0.91 | 0.6 | 0.12 | 0.51 | 0.4 | 0.2 | 0.06 | 0 | 0.03 | −2.27 |
| Cdc42ep1 | 21.91 | 1.61 | 6.12 | 1.45 | 2.62 | 24.79 | 9.56 | 5.77 | 11.61 | 16.59 | 13.39 | −2.26 |
| Zfpm2 | 10.04 | 0.24 | 1.48 | 1.17 | 0.26 | 0.81 | 1.6 | 0.07 | 0 | 0.03 | 0 | −2.25 |
| Ovol2 | 4.45 | 0 | 0.3 | 0 | 0.46 | 0.52 | 0.6 | 0.09 | 0 | 0 | 0 | −2.24 |
| Grhl2 | 4.62 | 0.02 | 0.33 | 0.05 | 0.13 | 1.76 | 1.97 | 0.08 | 0.05 | 0.05 | 0.03 | −2.24 |
| Gstt3 | 10.36 | 1.33 | 1.77 | 1.05 | 1.58 | 13.85 | 17.82 | 1.01 | 0.28 | 0.7 | 0.73 | −2.24 |
| Mia | 15.16 | 3.59 | 2.13 | 2.75 | 4.92 | 7.77 | 1.66 | 1.84 | 2.38 | 2.76 | 1.12 | −2.23 |
| C1qtnf2 | 6.64 | 0 | 1.01 | 0.28 | 1.31 | 14.9 | 6.37 | 0 | 0.08 | 0 | 0.14 | −2.22 |
| Scarf2 | 33.64 | 8.03 | 8.42 | 4.6 | 11.86 | 25.43 | 22.54 | 17.26 | 22.12 | 9.4 | 14.06 | −2.21 |
| Fbln2 | 9.86 | 1.37 | 1.78 | 0.93 | 2.52 | 5.95 | 7.86 | 3.44 | 3.85 | 2.81 | 3.76 | −2.21 |
| Cxcl13 | 6.26 | 0.08 | 0.72 | 0.43 | 0.24 | 0.85 | 0.63 | 0.42 | 0 | 0 | 0.14 | −2.20 |
| Cited4 | 12.61 | 1.24 | 2.74 | 1.18 | 1.18 | 4.58 | 4.3 | 0.04 | 0 | 0.4 | 0.77 | −2.20 |
| Prr15l | 5.16 | 0 | 0.54 | 0.14 | 0.27 | 9.88 | 15.67 | 0.04 | 0.14 | 0.07 | 0.12 | −2.20 |
| Vav3 | 7.27 | 0.25 | 1.32 | 0.28 | 0.36 | 3.43 | 8.15 | 3.96 | 2.4 | 1.83 | 1.79 | −2.20 |
| Eln | 8.5 | 0.77 | 1.73 | 0.41 | 1.63 | 16.14 | 25.14 | 0.56 | 0.42 | 0.05 | 0.23 | −2.20 |
| Stard10 | 36.63 | 8.72 | 8.64 | 5.82 | 10.55 | 42.08 | 33.71 | 7.75 | 12.03 | 20.64 | 18.22 | −2.19 |
| Ebf1 | 7.69 | 0.14 | 1.18 | 0.63 | 0.4 | 1.44 | 3.41 | 1.98 | 0.66 | 0.03 | 0.06 | −2.19 |
| Gap43 | 22.5 | 1.82 | 6.52 | 1.79 | 2.19 | 10.23 | 1.44 | 0.97 | 0.86 | 0.23 | 0.5 | −2.19 |
| Hbb-bs | 3.55 | 0.01 | 0 | 0 | 0.01 | 0.01 | 0 | 0 | 0 | 0.01 | 0.01 | −2.19 |
| Rnf128 | 7.1 | 0.12 | 0.85 | 0.74 | 0.19 | 4.74 | 26.5 | 5.87 | 3.99 | 3.7 | 1.63 | −2.17 |
| Eddm3b | 4.92 | 0.07 | 0.58 | 0.05 | 0.38 | 0.79 | 3.21 | 0 | 0.3 | 0.16 | 0 | −2.17 |
| Mdfi | 68.22 | 14.31 | 18.32 | 10.5 | 14.77 | 28.14 | 9.79 | 81.83 | 76.91 | 85.92 | 69.26 | −2.17 |
| Acta2 | 13.98 | 1.17 | 2.23 | 2.45 | 1.48 | 109.68 | 72.33 | 0.45 | 0.63 | 0.28 | 0.34 | −2.17 |
| Gli1 | 23.05 | 6.79 | 5.95 | 2.78 | 15.21 | 21.13 | 7.15 | 0 | 0.09 | 0.02 | 0.16 | −2.16 |
| Rab20 | 6.82 | 0.3 | 0.69 | 0.8 | 0.45 | 10.32 | 15.51 | 9.28 | 5.92 | 2.9 | 3.94 | −2.15 |
| Piezo2 | 7.83 | 0.32 | 1.4 | 0.55 | 0.67 | 3.04 | 2.18 | 0.64 | 0.49 | 0.13 | 0.25 | −2.15 |
| Cobll1 | 8.43 | 0.68 | 1.18 | 1.06 | 0.74 | 2.26 | 6.93 | 3.93 | 2.7 | 1.24 | 1.01 | −2.15 |
| Col12a1 | 5.26 | 0.15 | 0.54 | 0.28 | 0.55 | 5.38 | 7.62 | 0.82 | 0.7 | 0.51 | 0.24 | −2.15 |
| Fbln5 | 7.51 | 0.3 | 1.09 | 0.75 | 0.44 | 11.97 | 23.23 | 0.14 | 0.19 | 0.01 | 0.07 | −2.15 |
| Igfbp7 | 6.97 | 0.92 | 1.24 | 0.37 | 5.28 | 158.31 | 384.01 | 1.51 | 1.9 | 4.41 | 4.16 | −2.14 |
| Tcf15 | 15.49 | 0.84 | 4.63 | 0.84 | 1.82 | 6.95 | 2.8 | 1.71 | 0.97 | 1.03 | 1.12 | −2.14 |
| Cdh3 | 44.33 | 12.55 | 10.99 | 7.64 | 12.73 | 15.39 | 8.84 | 11.04 | 12.67 | 7.92 | 13.04 | −2.14 |
| Cyp2s1 | 5.25 | 0.48 | 0.54 | 0.31 | 0.71 | 9.19 | 14.36 | 2.67 | 4.69 | 2.76 | 1.97 | −2.13 |
| Igfbp2 | 213.25 | 75.5 | 60.87 | 34.83 | 107.52 | 143.05 | 71.03 | 133.13 | 104.11 | 88.62 | 86.44 | −2.13 |
| Dnm3os | 8.26 | 0.18 | 0.96 | 1.27 | 0.16 | 0.27 | 1.44 | 16.66 | 8.67 | 0.5 | 0.82 | −2.13 |
| Plagl1 | 90 | 1.62 | 23.71 | 15.91 | 2.99 | 5.86 | 27.43 | 37.04 | 6.18 | 1.52 | 1.97 | −2.13 |
| Col23a1 | 11.65 | 2.3 | 2.13 | 1.66 | 2.62 | 9.63 | 1.61 | 0.77 | 1.05 | 0.76 | 1.02 | −2.13 |
| Angpt1 | 7.47 | 0.06 | 1.15 | 0.74 | 0.19 | 1.08 | 3.7 | 0.67 | 0.42 | 0.04 | 0.23 | −2.12 |
| 2610528J11Rik | 3.81 | 0 | 0.17 | 0.04 | 0.55 | 7.11 | 20 | 0 | 0 | 0 | 0 | −2.12 |
| Dcdc2a | 4.37 | 0.13 | 0.34 | 0.14 | 0.36 | 2.05 | 7.14 | 0.02 | 0.08 | 0.04 | 0.04 | −2.11 |
| Arhgap24 | 11.79 | 1.3 | 2.49 | 1.43 | 2.42 | 9.45 | 21.5 | 2.17 | 1.55 | 2.04 | 1.07 | −2.11 |
| Pmp22 | 16.54 | 0.62 | 4.26 | 1.86 | 2.48 | 29.36 | 27.48 | 0.92 | 1.28 | 4.81 | 6.05 | −2.11 |
| Dact1 | 51.33 | 5.86 | 11.34 | 10.97 | 6.56 | 11.63 | 11.65 | 10.58 | 5.57 | 2.06 | 4.35 | −2.11 |
| Cyr61 | 927.94 | 60.67 | 223.37 | 208.8 | 101.29 | 202.23 | 339.41 | 21.21 | 10.53 | 138.91 | 170.27 | −2.10 |
| Fam212a | 36.93 | 6.8 | 11.31 | 4.43 | 7.16 | 21.59 | 10.87 | 8.93 | 3.12 | 0.49 | 1.75 | −2.10 |
| Tagln2 | 112.3 | 13.68 | 40.41 | 10.68 | 24.26 | 164.57 | 106.57 | 63.34 | 82.82 | 76.83 | 78.13 | −2.09 |
| Cela1 | 7.32 | 1.28 | 1.16 | 0.74 | 1.03 | 1.89 | 6.53 | 3.6 | 2.97 | 7.21 | 6.49 | −2.09 |
| Scube1 | 19.74 | 4.69 | 4.9 | 2.83 | 5.23 | 3.27 | 1.32 | 0.55 | 3.31 | 1.74 | 1.9 | −2.09 |
| Gipc2 | 6.46 | 0.43 | 0.9 | 0.6 | 0.59 | 6.8 | 11.07 | 0.67 | 0.59 | 0.87 | 1.16 | −2.09 |
| Amd1 | 3.29 | 0.03 | 0.01 | 0.01 | 0.03 | 0.03 | 0.01 | 0.02 | 0.03 | 0.03 | 0.05 | −2.09 |
| Itm2a | 18.1 | 1.93 | 4.28 | 2.72 | 2.39 | 24.2 | 16.86 | 0.56 | 0.33 | 0.35 | 0.1 | −2.09 |
| Ptx3 | 8.1 | 0.4 | 1.42 | 0.87 | 0.27 | 5.45 | 1.89 | 0.83 | 0.69 | 0.34 | 0.87 | −2.08 |
| Pxdc1 | 9.73 | 0.41 | 2.06 | 1.01 | 1.21 | 5.35 | 6.67 | 10.94 | 20.1 | 14.6 | 15.02 | −2.08 |
| Egfr | 4.86 | 0.07 | 0.54 | 0.25 | 0.38 | 1.73 | 1.6 | 1.08 | 0.78 | 0.13 | 0.11 | −2.07 |
| Ano4 | 4.73 | 0.35 | 0.46 | 0.27 | 0.28 | 1.84 | 0.41 | 0 | 0 | 0 | 0 | −2.07 |
| Elf3 | 7.89 | 0.97 | 1.31 | 0.95 | 1.74 | 22.34 | 13.41 | 18.3 | 9.21 | 10.43 | 10.36 | −2.06 |
| BC064078 | 4.48 | 0.12 | 0.47 | 0.17 | 0 | 3.62 | 12.46 | 0 | 0 | 0 | 0 | −2.05 |
| Trpv4 | 5.04 | 0.1 | 0.72 | 0.19 | 0.69 | 7.62 | 5.19 | 4.58 | 5.91 | 15.02 | 9.15 | −2.05 |
| Ntm | 6.42 | 0.53 | 1.06 | 0.52 | 1.26 | 2.18 | 0.78 | 4.39 | 1.91 | 0.31 | 0.42 | −2.05 |
| L1cam | 4.65 | 0.12 | 0.61 | 0.12 | 0.61 | 5.6 | 4.74 | 0.46 | 0.68 | 0.67 | 0.59 | −2.05 |
| Hs3st3b1 | 5.87 | 0.19 | 0.71 | 0.61 | 0.74 | 1.87 | 2.21 | 1.94 | 0.88 | 4.49 | 3.01 | −2.05 |
| Myl9 | 102.36 | 22.78 | 27.65 | 20.39 | 32.77 | 245.97 | 128.73 | 2.13 | 5.73 | 1.9 | 2.94 | −2.05 |
| Fn1 | 107.96 | 24.54 | 28.42 | 22.37 | 27.08 | 56.01 | 33.2 | 86.81 | 98.55 | 40.65 | 55.03 | −2.05 |
| Il33 | 13.01 | 0.95 | 2.77 | 2.03 | 2.17 | 15.05 | 42.85 | 53.45 | 19.49 | 69.79 | 36.29 | −2.04 |
| Anxa6 | 98.04 | 26.93 | 30.69 | 15.46 | 42.4 | 123.37 | 65.76 | 67.88 | 61.32 | 46.24 | 34.47 | −2.04 |
| Gucy1a3 | 7.61 | 0.29 | 1.27 | 0.92 | 0.68 | 8.98 | 31.34 | 0.79 | 0.37 | 0.02 | 0.12 | −2.04 |
| Gstt1 | 5.99 | 0.37 | 0.86 | 0.57 | 1.03 | 6.24 | 22.96 | 0.37 | 0.57 | 0.7 | 1.31 | −2.03 |
| Fgfr4 | 6.39 | 1.49 | 1.04 | 0.59 | 1.82 | 4.04 | 4.66 | 3.77 | 3.96 | 4.51 | 4.12 | −2.03 |
| Tox | 5.95 | 0.32 | 0.87 | 0.55 | 0.18 | 0.51 | 0.54 | 0.02 | 0 | 0 | 0 | −2.03 |
| Col27a1 | 6.62 | 0.8 | 1.22 | 0.53 | 1.59 | 4.17 | 4.05 | 0.74 | 0.36 | 0.82 | 0.6 | −2.02 |
| Gpm6b | 16.81 | 0.84 | 3.4 | 3.38 | 1.43 | 2.32 | 5.89 | 1.04 | 0.61 | 0.31 | 0.3 | −2.02 |
| Foxp2 | 7.51 | 0.16 | 0.98 | 1.22 | 0.24 | 0.49 | 0.47 | 0 | 0 | 0 | 0.02 | −2.02 |
| Cdc42ep5 | 23.08 | 5.93 | 7 | 2.9 | 7.41 | 33.88 | 17.78 | 3.24 | 4.29 | 2.91 | 6.39 | −2.02 |
| Vstm5 | 5.62 | 0.52 | 0.89 | 0.39 | 0.78 | 2.46 | 2.75 | 2.5 | 3 | 1.82 | 1.45 | −2.01 |
| Lrrc17 | 10.72 | 3.06 | 2.33 | 1.48 | 3.21 | 19.18 | 26.13 | 2.76 | 3.77 | 0.71 | 0.69 | −2.01 |
| Dpysl3 | 17.04 | 1.8 | 4.51 | 2.44 | 2.59 | 7.93 | 7.74 | 6.86 | 10.43 | 5.9 | 9.01 | −2.01 |
| Stfa3 | 3.03 | 0 | 0 | 0 | 0 | 0.71 | 10.87 | 0 | 0 | 0 | 1.96 | −2.01 |
| Tgfb2 | 13.38 | 1.24 | 3.17 | 1.97 | 1.58 | 1.82 | 3.11 | 3.88 | 2.8 | 2.27 | 1.69 | −2.01 |
| Epha7 | 15.57 | 1.34 | 3.03 | 3.2 | 1.83 | 2.62 | 2.29 | 0.26 | 0.07 | 0.06 | 0 | −2.01 |
| Tceal5 | 5.06 | 0.19 | 0.7 | 0.31 | 0.19 | 1.01 | 1.72 | 0.33 | 0.29 | 0.21 | 0.17 | −2.01 |
| Apoc1 | 20.76 | 1.68 | 6.79 | 2.06 | 1.36 | 19.21 | 77.01 | 0.98 | 2.41 | 0.37 | 0 | −2.00 |
| Mmp2 | 127.39 | 29.05 | 36.34 | 25.7 | 29.62 | 68.79 | 87.03 | 60.49 | 78.22 | 94.25 | 86.97 | −2.00 |
| Pygl | 13.49 | 1.99 | 3.2 | 2.04 | 2.43 | 11.78 | 20.55 | 21.56 | 19.54 | 16.1 | 15.09 | −2.00 |
| Prdm16 | 3.89 | 0.04 | 0.37 | 0.08 | 0.17 | 1.05 | 1.34 | 0.09 | 0.1 | 0.02 | 0.03 | −2.00 |
| Gca | 4.78 | 0.15 | 0.45 | 0.45 | 0.33 | 1.37 | 5.87 | 0.01 | 0 | 0 | 0 | −2.00 |
| Sfrp1 | 41.04 | 7.22 | 10.59 | 8.6 | 9.28 | 25.12 | 52.1 | 13.59 | 11.65 | 5.87 | 7.32 | −1.99 |
| Sphk1 | 4.66 | 0.23 | 0.42 | 0.44 | 0.58 | 2.76 | 3.93 | 0.5 | 1.18 | 1.81 | 1.23 | −1.98 |
| Prss8 | 13.75 | 2.22 | 3.57 | 1.89 | 2.49 | 22.48 | 27.07 | 1.02 | 1.19 | 1.71 | 0.49 | −1.98 |
| Pawr | 15.86 | 1.8 | 3.58 | 2.97 | 2.34 | 5.26 | 10.13 | 6.94 | 4.12 | 2.57 | 3.44 | −1.98 |
| Fam84b | 6.43 | 0.42 | 0.89 | 0.89 | 0.61 | 1.42 | 4.81 | 3.21 | 2.36 | 0.83 | 1.22 | −1.97 |
| Aldh1a7 | 3.75 | 0.12 | 0.27 | 0.15 | 0.04 | 2.2 | 1.2 | 0.05 | 0.34 | 0.45 | 0.29 | −1.97 |
| Pde1a | 4.43 | 0.13 | 0.41 | 0.36 | 0.58 | 1.98 | 6.33 | 0.36 | 0.42 | 0.04 | 0.74 | −1.97 |
| Prss23 | 7.74 | 0.48 | 1.48 | 0.99 | 1.05 | 4.84 | 12.31 | 0.04 | 0.05 | 0 | 0.14 | −1.97 |
| ligp1 | 9.33 | 0.36 | 1.41 | 1.88 | 0.43 | 0.71 | 4.08 | 0.7 | 1.7 | 0.85 | 0.45 | −1.97 |
| Arhgap8 | 7 | 1.34 | 1.36 | 0.74 | 1.77 | 4.46 | 6.11 | 0.51 | 1.83 | 1.01 | 1.76 | −1.96 |
| Fstl1 | 193.48 | 40.61 | 51.11 | 47.22 | 55.74 | 161.07 | 211.84 | 84.57 | 81.14 | 45.04 | 40.39 | −1.95 |
| Amigo2 | 19.87 | 3.32 | 4.64 | 4.13 | 3.12 | 5.58 | 5.93 | 1.23 | 1.12 | 1.3 | 1.39 | −1.95 |
| St6galnac2 | 10.29 | 2.27 | 2.36 | 1.47 | 2.08 | 8.33 | 6.58 | 1.87 | 2.11 | 6.25 | 3.87 | −1.95 |
| Col6a3 | 4.48 | 0.29 | 0.53 | 0.3 | 0.84 | 7.05 | 6.47 | 1.48 | 0.46 | 0.27 | 0.39 | −1.95 |
| Mpzl2 | 6.68 | 0.46 | 1.12 | 0.86 | 0.36 | 4.69 | 6.99 | 2.28 | 2.33 | 2.02 | 3.35 | −1.95 |
| Hapln1 | 7.08 | 0 | 1.81 | 0.38 | 0.11 | 1.62 | 0.58 | 0 | 0 | 0 | 0 | −1.95 |
| Glipr2 | 36.27 | 4.78 | 9.65 | 7.69 | 7.74 | 26.86 | 17.44 | 75.41 | 66.89 | 32.63 | 40.58 | −1.95 |
| Nsg2 | 4.89 | 0.24 | 0.87 | 0.2 | 0.72 | 2.78 | 6.59 | 0.07 | 0.15 | 0.13 | 0.07 | −1.94 |
| Gng11 | 56.74 | 5.34 | 21.61 | 6.49 | 9.58 | 61.48 | 37.96 | 7.4 | 3.73 | 0.4 | 0.86 | −1.94 |
| Nid1 | 26.08 | 3.21 | 7.59 | 4.53 | 4.11 | 25.01 | 49.76 | 22.72 | 24.92 | 14.71 | 12.89 | −1.94 |
| Lum | 5.02 | 0 | 0.67 | 0.47 | 0.46 | 9.33 | 2.42 | 0.14 | 0.04 | 0.04 | 0.07 | −1.94 |
| Kel | 2.83 | 0 | 0 | 0 | 0.03 | 0.13 | 0.69 | 0.02 | 0 | 0 | 0 | −1.94 |
| Dcn | 10.9 | 1.35 | 2.36 | 1.88 | 2.44 | 47.86 | 28.7 | 12.52 | 7.08 | 2.05 | 0.86 | −1.93 |
| 9230102O04Rik | 3.67 | 0 | 0.45 | 0 | 0.44 | 3.36 | 5.83 | 0 | 0 | 0 | 0 | −1.93 |
| Ptpn3 | 5.93 | 0.62 | 0.91 | 0.73 | 0.78 | 1.62 | 3.01 | 6.65 | 6.1 | 6.75 | 5.59 | −1.93 |
| Kit | 12.05 | 1.21 | 2.83 | 2.04 | 1.42 | 4.81 | 3.73 | 1.91 | 1.82 | 0.64 | 1.62 | −1.93 |
| Coro2b | 16.95 | 3.4 | 5.33 | 2.17 | 4.86 | 13.48 | 4.33 | 6.14 | 6.15 | 3.09 | 4.08 | −1.92 |
| Npnt | 29.3 | 6.55 | 6.55 | 7.5 | 5 | 23.34 | 30.16 | 0.43 | 0.48 | 1.11 | 1.35 | −1.92 |
| Sim1 | 3.09 | 0 | 0.15 | 0.02 | 0.13 | 0.24 | 1.14 | 0 | 0 | 0 | 0 | −1.91 |
| Adcy4 | 7.42 | 0.41 | 2.09 | 0.38 | 1.05 | 6.61 | 2.44 | 4.31 | 2.37 | 1.02 | 1.15 | −1.91 |
| Rftn2 | 5.89 | 0.13 | 1.25 | 0.41 | 0.34 | 3.25 | 1.82 | 0.35 | 0.16 | 0.07 | 0.08 | −1.91 |
| Rxra | 19.86 | 5.01 | 5.7 | 3.39 | 6.41 | 13.68 | 8.52 | 6.96 | 9.94 | 12.85 | 11.71 | −1.91 |
| 9030625G05Rik | 3.15 | 0 | 0.21 | 0 | 0 | 0.88 | 1.79 | 0.11 | 0.09 | 0 | 0 | −1.91 |
| Lhx1os | 12.98 | 1.64 | 4.4 | 1.05 | 4.99 | 9.55 | 19.22 | 0 | 0 | 0 | 0.65 | −1.91 |
| Sept5 | 37.13 | 10.65 | 11.42 | 6.97 | 11.95 | 20.55 | 10.26 | 75.43 | 146.42 | 75.73 | 117.73 | −1.90 |
| Fbxl7 | 15.71 | 2.19 | 3.93 | 3.01 | 2.8 | 4.8 | 3.52 | 0.03 | 0.04 | 0.43 | 0.03 | −1.90 |
| Mgll | 5.66 | 0.32 | 1.3 | 0.27 | 0.58 | 5.32 | 5.28 | 0.4 | 0.34 | 1.3 | 0.98 | −1.90 |
| 0610040J01Rik | 11.89 | 1.15 | 2.74 | 2.18 | 2.66 | 10.08 | 15.99 | 1.55 | 2.13 | 6.46 | 4.33 | −1.90 |
| Fzd1 | 45.66 | 7.32 | 10.68 | 12.39 | 6.03 | 16.81 | 19.69 | 13.24 | 14.71 | 8.62 | 15.17 | −1.90 |
| 2900092D14Rik | 3.87 | 0 | 0.55 | 0.07 | 0.12 | 0.56 | 4.02 | 0 | 0 | 0 | 0 | −1.89 |
| Mpo | 2.73 | 0 | 0.01 | 0 | 0 | 0 | 0 | 0 | 0 | 0 | 0 | −1.89 |
| Emcn | 47.14 | 0.73 | 23.06 | 0.91 | 2.59 | 89.71 | 20.56 | 4.43 | 3.51 | 0.62 | 1.12 | −1.89 |
| Adam19 | 19.27 | 3.42 | 5.54 | 3.4 | 5.29 | 10.18 | 6.75 | 14.03 | 11.07 | 4.65 | 4.31 | −1.89 |
| Glb1l2 | 6.46 | 0.9 | 1.22 | 0.81 | 1.36 | 9.54 | 9.46 | 0.04 | 0.05 | 0.17 | 0.16 | −1.89 |
| Cd82 | 13.28 | 2.38 | 3.7 | 2.02 | 3.55 | 26.66 | 47.5 | 32.29 | 43.54 | 75.52 | 61.84 | −1.89 |
| Lrp1 | 12.79 | 3.1 | 3.47 | 1.99 | 4.05 | 9.85 | 8.23 | 11.51 | 10.81 | 9.11 | 8.48 | −1.89 |
| Emilin1 | 128.43 | 50.45 | 40.89 | 27.13 | 51.65 | 100.44 | 48.94 | 68.85 | 75.79 | 19.55 | 31.58 | −1.89 |
| Runx1t1 | 4.6 | 0.13 | 0.61 | 0.42 | 0.24 | 0.11 | 0.35 | 0 | 0 | 0 | 0 | −1.89 |
| Epha1 | 5.7 | 0.59 | 1.04 | 0.59 | 0.71 | 3.8 | 1.58 | 0.67 | 0.96 | 0.43 | 0.62 | −1.88 |
| Ngp | 2.69 | 0 | 0 | 0 | 0 | 0.08 | 1.34 | 0 | 0 | 0 | 0 | −1.88 |
| Ace2 | 6.14 | 0.33 | 1.27 | 0.6 | 0.48 | 12.44 | 16.41 | 0 | 0 | 0 | 0 | −1.88 |
| Aplnr | 20.39 | 0.09 | 9.21 | 0.39 | 0.86 | 39.81 | 5.58 | 0.03 | 0.02 | 0.05 | 0.04 | −1.88 |
| Gata5 | 4.96 | 0.2 | 0.81 | 0.43 | 0.4 | 1.25 | 0.17 | 0.07 | 0.2 | 0.05 | 0.09 | −1.88 |
| Cldn5 | 26.47 | 1.2 | 12.57 | 0.37 | 2.95 | 65.05 | 8.53 | 0.04 | 0 | 0 | 0 | −1.88 |
| Rxrg | 3.72 | 0.31 | 0.42 | 0.15 | 0.37 | 0.45 | 0.51 | 0.03 | 0.16 | 0 | 0 | −1.88 |
| Frk | 8.53 | 0.43 | 1.61 | 1.58 | 0.98 | 1.57 | 4.08 | 2.43 | 1.35 | 1.05 | 1.38 | −1.88 |
| Ap1s3 | 6.96 | 0.17 | 1.29 | 1.05 | 0.55 | 0.92 | 1.92 | 2.08 | 1.03 | 2.62 | 1.27 | −1.88 |
| Dact2 | 14.12 | 2.39 | 4.44 | 1.82 | 2.47 | 13.69 | 4.27 | 1.15 | 2.96 | 10.38 | 11.5 | −1.87 |
| Rapgef3 | 6.18 | 0.37 | 1.68 | 0.25 | 0.94 | 9.11 | 3.85 | 4.38 | 1.72 | 4.68 | 3.5 | −1.87 |
| Mgp | 74.47 | 20 | 21.43 | 17.88 | 20.24 | 97.21 | 619.58 | 190.96 | 174.21 | 97.1 | 105.42 | −1.87 |
| Emp1 | 11.45 | 0.64 | 3.97 | 0.85 | 2.04 | 38.97 | 45.05 | 5.59 | 2.44 | 1.86 | 1.19 | −1.87 |
| Sec24d | 9.93 | 1.11 | 2.6 | 1.39 | 3.04 | 10.36 | 9.68 | 18.7 | 20.23 | 11.44 | 9.8 | −1.87 |
| Ramp3 | 10.72 | 2.64 | 3.34 | 1.09 | 3.74 | 10.52 | 5.82 | 2.23 | 2.01 | 0.66 | 1.2 | −1.87 |
| Npr3 | 4.02 | 0.15 | 0.55 | 0.21 | 0.79 | 1.88 | 0.85 | 0.01 | 0.22 | 0.16 | 0.58 | −1.86 |
| Infsf13b | 5.5 | 0.41 | 0.82 | 0.76 | 0.78 | 2.88 | 5.35 | 0.03 | 0.21 | 0.11 | 0.46 | −1.86 |
| AW549542 | 3.43 | 0.1 | 0.3 | 0.14 | 0.5 | 3.89 | 3.62 | 0 | 0 | 0 | 0 | −1.86 |
| Cacna1g | 8.61 | 1.95 | 2.24 | 1.06 | 2.74 | 6.21 | 3.16 | 6.6 | 3.35 | 2.5 | 1.82 | −1.86 |
| Gimap4 | 13.28 | 0.61 | 5.61 | 0.28 | 0.78 | 47.45 | 11.58 | 0 | 0 | 0 | 0 | −1.86 |
| Col25a1 | 4.49 | 0.15 | 0.54 | 0.5 | 0.09 | 0.5 | 0.37 | 0.5 | 0.17 | 0.04 | 0.12 | −1.85 |
| Tfcp2l1 | 3.35 | 0.13 | 0.35 | 0.06 | 0.51 | 6.5 | 10.99 | 0 | 0.03 | 0.01 | 0.02 | −1.85 |
| Ephb3 | 8.38 | 2.24 | 1.87 | 1.34 | 2.52 | 5.47 | 3.95 | 10.63 | 10.06 | 6.01 | 6.53 | −1.85 |
| Ebf2 | 3.69 | 0.04 | 0.35 | 0.27 | 0.25 | 0.03 | 0.15 | 0.01 | 0 | 0 | 0.05 | −1.84 |
| Sulf1 | 13.66 | 2.41 | 3.4 | 2.79 | 2.5 | 2.92 | 7.35 | 2.75 | 6.49 | 8.34 | 10.17 | −1.84 |
| Lingo1 | 9.71 | 1.54 | 2.43 | 1.57 | 2.64 | 4.86 | 2.36 | 7.62 | 10.01 | 11.9 | 8.55 | −1.84 |
| Lama1 | 7.91 | 2.47 | 1.83 | 1.17 | 3.78 | 4.48 | 5.72 | 0.95 | 2.64 | 3.41 | 3.77 | −1.83 |
| Slc7a8 | 6.42 | 0.38 | 1.69 | 0.48 | 1.01 | 7.94 | 10.6 | 0.14 | 0.18 | 0.07 | 2.03 | −1.83 |
| Dmtn | 4.6 | 0.23 | 0.96 | 0.19 | 0.45 | 4.17 | 8.94 | 0.13 | 0.26 | 0.16 | 0.37 | −1.83 |
| Mamdc2 | 5.41 | 0.37 | 0.73 | 0.88 | 0.2 | 0.17 | 1.31 | 1.26 | 1.73 | 0.21 | 0.13 | −1.83 |
| Lypd6b | 3.56 | 0.26 | 0.42 | 0.15 | 0.6 | 5.04 | 2.25 | 0.02 | 0.08 | 0.04 | 0.2 | −1.83 |
| Ntrk2 | 4.81 | 0.29 | 0.69 | 0.59 | 0.26 | 0.83 | 1.41 | 3.89 | 4.13 | 0.38 | 1.35 | −1.82 |
| Srxn1 | 6.28 | 0.62 | 1.38 | 0.73 | 0.92 | 5.58 | 7.97 | 2.54 | 4.41 | 8.35 | 5.01 | −1.82 |
| Lima1 | 25.95 | 6.26 | 6.89 | 6.34 | 8.81 | 21.81 | 19.17 | 28.12 | 22.79 | 19.11 | 17.02 | −1.82 |
| Gli2 | 10.16 | 1.78 | 2.64 | 1.67 | 2.54 | 3.42 | 1.62 | 1.94 | 4.28 | 1.89 | 3.92 | −1.82 |
| Itga3 | 10.47 | 2.76 | 2.91 | 1.59 | 5.65 | 16.69 | 9.13 | 3.87 | 12.66 | 69.9 | 50.76 | −1.82 |
| Spink8 | 3.4 | 0 | 0.5 | 0 | 0.33 | 11.42 | 61.7 | 0 | 0 | 0 | 0 | −1.82 |
| Mfsd2a | 10.17 | 2.13 | 2.64 | 1.72 | 2.27 | 2.14 | 0.35 | 4.71 | 2.59 | 3.6 | 4.69 | −1.81 |
| Emx2os | 3.26 | 0 | 0.39 | 0.04 | 0.21 | 0.93 | 0.98 | 0 | 0.11 | 0.68 | 0.68 | −1.81 |
| Cys1 | 2.98 | 0.29 | 0.26 | 0.02 | 0.81 | 6.06 | 9.67 | 0.08 | 0.24 | 0.16 | 0.09 | −1.80 |
| Mertk | 6.47 | 0.53 | 1.34 | 0.94 | 0.97 | 2.84 | 4.4 | 1.12 | 1.06 | 0.35 | 0.33 | −1.80 |
| Ltbp3 | 14.16 | 4.3 | 4.4 | 2.31 | 6.87 | 16.48 | 14.59 | 7.37 | 7.87 | 19.37 | 12.49 | −1.80 |
| Fam84a | 4.96 | 0.14 | 0.71 | 0.73 | 0.59 | 1.22 | 1.89 | 0.03 | 0.02 | 0.03 | 0 | −1.79 |
| Mrvi1 | 4.3 | 0.12 | 0.7 | 0.36 | 0.34 | 3.19 | 3 | 0.03 | 0.08 | 0 | 0 | −1.79 |
| Mir130c | 966.64 | 121.86 | 418.21 | 138.49 | 102.72 | 1232.44 | 375.59 | 377.08 | 1404.67 | 484.51 | 1200.48 | −1.79 |
| Maged2 | 307.69 | 101.02 | 99.09 | 77.26 | 131.55 | 221.71 | 120.33 | 71.11 | 81.75 | 73.89 | 63.23 | −1.79 |
| Fez1 | 6.06 | 2.27 | 1.46 | 0.62 | 3.45 | 3.64 | 1.99 | 0.49 | 0.36 | 0.31 | 0.25 | −1.79 |
| Lad1 | 9 | 2.28 | 2.47 | 1.31 | 4.68 | 13.65 | 6.61 | 2.84 | 5.73 | 8.11 | 7.57 | −1.79 |
| Erbb3 | 2.84 | 0.1 | 0.18 | 0.04 | 0.16 | 0.84 | 0.67 | 0.01 | 0.06 | 0.22 | 0.07 | −1.79 |
| Fxyd7 | 2.89 | 0 | 0.25 | 0 | 1.47 | 8.62 | 1.51 | 1.69 | 1.33 | 0 | 0.57 | −1.79 |
| Dbh | 5.12 | 0.07 | 1.29 | 0.25 | 0 | 0.07 | 0 | 0 | 0 | 0 | 0.07 | −1.79 |
| Camp | 2.57 | 0.19 | 0 | 0.07 | 0 | 0 | 1.15 | 0 | 0.2 | 0 | 0 | −1.79 |
| Grasp | 14.84 | 3.43 | 4.86 | 2.34 | 3.82 | 11.17 | 4.46 | 8.83 | 4 | 4.29 | 4.06 | −1.78 |
| Mterfd2 | 57.59 | 14.6 | 17.57 | 14.46 | 15.1 | 20.35 | 13.55 | 15.44 | 15.86 | 9.41 | 14.18 | −1.78 |
| Spon2 | 4.73 | 0.57 | 0.88 | 0.45 | 0.95 | 3.07 | 1.69 | 0.44 | 1.16 | 0.82 | 0.6 | −1.78 |
| Dkk3 | 4.88 | 0.63 | 0.89 | 0.53 | 0.86 | 5.52 | 7.18 | 3.76 | 6.34 | 5.42 | 5.14 | −1.78 |
| Capg | 22.41 | 5.89 | 6.98 | 4.67 | 8.04 | 47.16 | 30.53 | 32.35 | 26.25 | 51.03 | 27.54 | −1.78 |
| Tmtc4 | 33.6 | 4.43 | 8.92 | 9.3 | 5.97 | 6.83 | 8.61 | 4.84 | 3.93 | 3.93 | 5.04 | −1.77 |
| Crispld2 | 10.63 | 1.81 | 2.37 | 2.43 | 0.87 | 1.15 | 4.53 | 5.91 | 8.83 | 2.3 | 1.97 | −1.77 |
| Gsdmd | 6.07 | 0.83 | 1.38 | 0.76 | 1.78 | 8.41 | 9.79 | 3.88 | 3.25 | 1.75 | 1.99 | −1.77 |
| Slc24a3 | 5.4 | 0.74 | 1.11 | 0.64 | 0.75 | 1.82 | 1.43 | 0 | 0 | 0.08 | 0.04 | −1.77 |
| Dkk1 | 4.97 | 0.23 | 0.66 | 0.84 | 0.95 | 4.75 | 7.24 | 0.04 | 0.15 | 0 | 0.6 | −1.77 |
| Atp9a | 13.06 | 4.06 | 4.15 | 2.1 | 5.32 | 10.98 | 6.72 | 1.26 | 1.06 | 1.15 | 1.13 | −1.77 |
| Ptrf | 13.8 | 0.97 | 5.84 | 0.85 | 2.47 | 30.58 | 21.56 | 15.01 | 14.1 | 23.2 | 14.46 | −1.77 |
| Sh2d3c | 14.94 | 2.57 | 5.65 | 1.73 | 2.7 | 16.41 | 4.71 | 3.47 | 2.93 | 1.17 | 1.07 | −1.76 |
| Mapk15 | 6.14 | 1.6 | 1.38 | 0.83 | 2.2 | 4 | 5.12 | 1.68 | 1.23 | 1.02 | 1.1 | −1.76 |
| Rtn4r | 10.76 | 4.09 | 3.09 | 1.86 | 5.63 | 4.41 | 0.95 | 4.96 | 4.9 | 1.8 | 1.79 | −1.76 |
| Epb4.1l3 | 19.55 | 2.54 | 5.89 | 4.26 | 4.02 | 3.99 | 3.53 | 0 | 0.02 | 0.04 | 0.04 | −1.76 |
| Emp3 | 70.97 | 25.11 | 24.97 | 15.91 | 51.69 | 110.73 | 81.45 | 132.14 | 88.73 | 85.09 | 52.33 | −1.75 |
| E330013P04Rik | 3.27 | 0.16 | 0.43 | 0.12 | 0.42 | 1.39 | 0.36 | 0.21 | 0.21 | 1.03 | 1.97 | −1.74 |
| Lix1 | 5.48 | 0.48 | 0.97 | 0.9 | 0.88 | 1.54 | 3.79 | 0.58 | 0.81 | 0.86 | 1.88 | −1.74 |
| Rgl3 | 13.47 | 4.38 | 3.89 | 2.81 | 5.86 | 18.37 | 10.36 | 11.92 | 12.58 | 15.01 | 15.73 | −1.73 |
| Kcnd2 | 3.43 | 0.05 | 0.43 | 0.24 | 0.18 | 0.38 | 0.53 | 0.03 | 0 | 0.04 | 0 | −1.73 |
| Col8a2 | 5.02 | 0.87 | 1.11 | 0.53 | 1.19 | 8.5 | 5.12 | 0.38 | 0.76 | 0.51 | 0.49 | −1.73 |
| Cnksr1 | 4.19 | 0.78 | 0.72 | 0.42 | 0.72 | 4.04 | 3.85 | 0.2 | 0.21 | 1.55 | 0.72 | −1.72 |
| Gm11627 | 8.98 | 2.43 | 2.4 | 1.64 | 1.64 | 7.22 | 5.18 | 24.56 | 13.8 | 6.52 | 3.74 | −1.72 |
| Marveld1 | 23.85 | 6.65 | 7.65 | 5.41 | 10 | 24.55 | 21.17 | 21.42 | 25.83 | 22.53 | 13.89 | −1.72 |
| Sardh | 9.18 | 3.35 | 2.69 | 1.48 | 6.06 | 13.26 | 10.12 | 9.57 | 9.18 | 18.22 | 12.51 | −1.72 |
| Cystm1 | 6.44 | 1.77 | 1.52 | 0.99 | 1.79 | 31.15 | 30.29 | 0.46 | 4.02 | 20.9 | 10.44 | −1.72 |
| Syndig1 | 3.76 | 0.33 | 0.37 | 0.52 | 0.63 | 0.8 | 0.55 | 0 | 0 | 0 | 0 | −1.72 |
| Igdcc4 | 12.45 | 1.34 | 3.15 | 3.02 | 1.79 | 3.28 | 3.72 | 11.41 | 9.04 | 6.19 | 6.6 | −1.72 |
| Gdf5 | 4.36 | 0.18 | 0.64 | 0.63 | 0.18 | 0.04 | 0.09 | 0.8 | 0.3 | 0.36 | 0.26 | −1.71 |
| Nav1 | 7.45 | 1.03 | 2.01 | 1.15 | 1.5 | 3.6 | 3.09 | 3.94 | 3.62 | 3.51 | 3.82 | −1.71 |
| Atf3 | 100.8 | 13.81 | 32.87 | 27.36 | 11.79 | 62.27 | 80.52 | 11.43 | 6.84 | 17.04 | 20.28 | −1.71 |
| Naalad2 | 3.51 | 0.15 | 0.54 | 0.22 | 0.31 | 2.27 | 3.66 | 0.02 | 0.06 | 0 | 0 | −1.71 |
| Adamtsl4 | 3.77 | 0.36 | 0.6 | 0.32 | 0.55 | 2.08 | 1.14 | 0.27 | 0.32 | 0.46 | 0.56 | −1.71 |
| Serinc2 | 46.34 | 21.97 | 16.37 | 10.62 | 18.48 | 28.75 | 12.58 | 9.16 | 10.92 | 8.18 | 8.39 | −1.71 |
| Acp5 | 4.74 | 0.67 | 1.01 | 0.51 | 0.74 | 4.01 | 6.27 | 1.55 | 1.44 | 1.67 | 0.95 | −1.71 |
| Sorcs1 | 2.97 | 0.17 | 0.3 | 0.14 | 0.18 | 0.67 | 0.87 | 0.02 | 0.25 | 0.07 | 0.1 | −1.70 |
| Tm4sf1 | 4.24 | 0.18 | 0.77 | 0.46 | 0.96 | 19.47 | 16 | 3.25 | 1.95 | 1.42 | 2 | −1.70 |
| Sox8 | 9.09 | 1.84 | 2.32 | 1.9 | 1.47 | 0.8 | 0.68 | 0.78 | 0.59 | 0.48 | 0.58 | −1.70 |
| Dock10 | 3.26 | 0.01 | 0.47 | 0.16 | 0.1 | 0.53 | 1.15 | 0 | 0 | 0.01 | 0 | −1.70 |
| Tshz3 | 5.44 | 0.24 | 0.96 | 1.02 | 0.36 | 2.37 | 1.21 | 0.59 | 0.46 | 0.83 | 0.88 | −1.69 |
| Prrx2 | 22.19 | 5.47 | 7.35 | 5 | 6.38 | 10.52 | 6.85 | 128.65 | 144.51 | 249.16 | 199.42 | −1.69 |
| Frem2 | 15.68 | 4.09 | 4.03 | 4.3 | 4.26 | 5.73 | 4.24 | 1.76 | 3.53 | 4.49 | 4.33 | −1.69 |
| Gucy1b3 | 4.02 | 0.2 | 0.65 | 0.46 | 0.54 | 4.59 | 7.96 | 0.96 | 0.13 | 0.03 | 0.04 | −1.69 |
| Pcdh18 | 18.32 | 2.52 | 4.77 | 5.21 | 3.32 | 6.26 | 8.07 | 13 | 11.07 | 5.01 | 5.46 | −1.69 |
| Sparcl1 | 12.09 | 0.98 | 4.95 | 1.17 | 1.2 | 15.95 | 27.95 | 0.12 | 0.27 | 0.09 | 0.14 | −1.69 |
| Gpr56 | 6.51 | 0.87 | 1.68 | 0.98 | 1.72 | 18.94 | 31.42 | 0.11 | 0.12 | 0.35 | 0.61 | −1.69 |
| Gabra3 | 4.01 | 0.24 | 0.62 | 0.49 | 0.15 | 0.72 | 0.71 | 2.72 | 1.01 | 0.19 | 0.27 | −1.69 |
| Pcdh1 | 4.39 | 0.06 | 1.27 | 0.08 | 0.42 | 3.76 | 0.84 | 6.55 | 5.83 | 8.28 | 5.08 | −1.69 |
| Ros1 | 2.49 | 0 | 0.17 | 0 | 0.03 | 0.28 | 0.33 | 0.01 | 0 | 0 | 0 | −1.69 |
| Il2rg | 7.32 | 0.87 | 2.31 | 0.87 | 0.94 | 14.2 | 5.57 | 0.03 | 0.34 | 0.06 | 0.1 | −1.69 |
| Cnn2 | 216.18 | 53.88 | 69.99 | 63.42 | 64.81 | 193.4 | 98.45 | 66.81 | 95.64 | 58.05 | 63.93 | −1.69 |
| S100a10 | 262.72 | 92.01 | 87.16 | 75.32 | 107.21 | 471.8 | 219.88 | 352.34 | 351.45 | 760.58 | 453.43 | −1.68 |
| Cyp26b1 | 7.93 | 0.53 | 2.86 | 0.71 | 0.47 | 3.6 | 1.15 | 0.01 | 0.05 | 0.02 | 0.12 | −1.68 |
| Gfra3 | 8.52 | 1.37 | 2.05 | 1.89 | 1.61 | 1.04 | 0.36 | 2.17 | 3.65 | 0 | 0.99 | −1.68 |
| Sostdc1 | 2.83 | 0 | 0.25 | 0.14 | 0.15 | 16.33 | 178.45 | 0 | 0 | 0 | 0 | −1.68 |
| Rtn4rl1 | 3.06 | 0.05 | 0.41 | 0.13 | 0.23 | 1.87 | 1.22 | 0.14 | 0.13 | 0.08 | 0.04 | −1.68 |
| Snai1 | 37.77 | 8.99 | 13.75 | 8.54 | 10.45 | 14.19 | 5.11 | 12.05 | 14.8 | 5.37 | 7.09 | −1.67 |
| Sall4 | 2.78 | 0.03 | 0.26 | 0.11 | 0.03 | 0 | 0 | 0.32 | 0.16 | 0 | 0 | −1.67 |
| Kcnk1 | 13.81 | 3.66 | 3.92 | 3.38 | 3.7 | 12 | 12.85 | 2.86 | 9.93 | 17.13 | 12.78 | −1.67 |
| Tinagl1 | 6.19 | 0.39 | 2.3 | 0.22 | 0.98 | 31.46 | 19.86 | 15.98 | 32.73 | 25.12 | 30.03 | −1.67 |
| Cxcl12 | 218.7 | 53.44 | 64.1 | 72.98 | 30.21 | 33.94 | 86.39 | 29.2 | 66.26 | 27.24 | 22.69 | −1.66 |
| Itpr3 | 4.18 | 0.55 | 1.02 | 0.26 | 0.98 | 4.47 | 1.73 | 8.24 | 11.08 | 8.77 | 7.28 | −1.66 |
| Smtn | 26.43 | 7.96 | 10.59 | 4.78 | 9.47 | 23.28 | 11.38 | 12.75 | 12.16 | 13.96 | 10.83 | −1.66 |
| Ldoc1 | 3.56 | 0.14 | 0.74 | 0.15 | 0.48 | 1.31 | 0.07 | 0 | 0 | 0 | 0 | −1.66 |
| 4931403E22Rik | 5.67 | 0.99 | 1.22 | 1.01 | 1.34 | 3.84 | 1.95 | 1.06 | 0 | 0.54 | 0.59 | −1.66 |
| Penk | 4.81 | 0.83 | 0.64 | 1.05 | 3.34 | 242.72 | 175.73 | 0.07 | 0 | 0.14 | 0 | −1.65 |
| Kifc3 | 8.54 | 1.89 | 2.98 | 1.08 | 3.61 | 12.75 | 15.36 | 3.91 | 3.62 | 5.93 | 4.35 | −1.65 |
| Hgf | 3.99 | 0.05 | 0.62 | 0.55 | 0.65 | 0.31 | 1 | 1.56 | 0.88 | 0.31 | 0.06 | −1.65 |
| Arap3 | 9.96 | 1.09 | 4.41 | 0.56 | 1.28 | 15.59 | 3.45 | 1.83 | 1.65 | 1.03 | 0.71 | −1.65 |
| Capn5 | 5.28 | 0.58 | 1.31 | 0.69 | 1.51 | 7.74 | 5.51 | 0.38 | 0.86 | 0.75 | 0.71 | −1.65 |
| Clca1 | 4.41 | 0.05 | 0.61 | 0.84 | 0.19 | 3.51 | 13.42 | 0.06 | 0.03 | 0 | 0.05 | −1.65 |
| Vldlr | 5.86 | 0.52 | 1.19 | 1.2 | 0.93 | 3.7 | 12.03 | 19.81 | 17.2 | 15.94 | 12.91 | −1.64 |
| Sema3c | 14.33 | 0.97 | 3.28 | 4.54 | 1.29 | 6.23 | 13.58 | 13.49 | 8.61 | 13.31 | 13.41 | −1.64 |
| Edil3 | 3.82 | 0.04 | 0.44 | 0.65 | 0.15 | 0.35 | 0.11 | 0.22 | 0.02 | 0 | 0 | −1.64 |
| Ccnd1 | 93.42 | 30.19 | 40.02 | 18.55 | 62.49 | 73.78 | 55.26 | 1.92 | 4.53 | 3.23 | 8.23 | −1.64 |
| Tubb3 | 6.6 | 2.02 | 2.06 | 0.82 | 1.48 | 0.44 | 0.02 | 0.85 | 2.18 | 0.22 | 2.69 | −1.64 |
| Wnt5a | 5.96 | 0.48 | 1.49 | 0.98 | 0.55 | 4.45 | 6.27 | 21.91 | 16.74 | 7.46 | 7.95 | −1.64 |
| Olfml1 | 11.97 | 4.3 | 3.19 | 3.14 | 4.62 | 11.83 | 15.59 | 3.28 | 8.83 | 0.95 | 2.62 | −1.64 |
| Lphn3 | 4.43 | 0.57 | 0.86 | 0.63 | 0.66 | 0.87 | 1.23 | 0.53 | 0.44 | 0 | 0.07 | −1.64 |
| Iglon5 | 15.41 | 5.42 | 5.38 | 3.17 | 5.98 | 6.23 | 1.87 | 0.3 | 0.3 | 1.2 | 1.27 | −1.64 |
| Mdfic | 8.25 | 0.67 | 1.98 | 1.97 | 0.62 | 2.58 | 6.65 | 1.98 | 1.41 | 1.19 | 0.55 | −1.64 |
| Hs3st3a1 | 2.96 | 0 | 0.27 | 0.28 | 0.03 | 0.42 | 0.36 | 0.71 | 0.45 | 0.84 | 0.72 | −1.64 |
| Anxa5 | 87.76 | 22.03 | 32.51 | 22.85 | 24.28 | 101.16 | 103.96 | 239.62 | 225.95 | 281.9 | 221.3 | −1.63 |
| Stfa2l1 | 2.09 | 0 | 0 | 0 | 0 | 0.71 | 12.38 | 0 | 0 | 0 | 0 | −1.63 |
| Gm20755 | 2.5 | 0.14 | 0.2 | 0.07 | 0 | 0.18 | 0.23 | 0.03 | 0 | 0 | 0.17 | −1.62 |
| Pde9a | 35.34 | 12.83 | 11.74 | 9.83 | 11.99 | 13.63 | 9.72 | 3.57 | 5.25 | 2.47 | 2.42 | −1.62 |
| Emilin2 | 4.7 | 0.91 | 1.13 | 0.57 | 1.13 | 1.99 | 3.04 | 0.78 | 1.08 | 0.47 | 0.92 | −1.62 |
| Thra | 27.15 | 11.05 | 9.92 | 6.36 | 12.7 | 30.2 | 21.75 | 12.34 | 11.06 | 10.01 | 9.67 | −1.62 |
| Galnt14 | 5.05 | 0.95 | 1.01 | 0.92 | 1.51 | 4.33 | 4.59 | 1.4 | 2.02 | 1.77 | 1.44 | −1.62 |
| Pear1 | 12.43 | 3.76 | 4.63 | 2.1 | 5.73 | 19.76 | 15.11 | 7.82 | 4.3 | 6.53 | 5.73 | −1.62 |
| Scnn1g | 2.26 | 0 | 0.11 | 0.01 | 0.25 | 15.88 | 16.24 | 0 | 0.03 | 0 | 0 | −1.62 |
| Adamts1 | 47.79 | 6.49 | 14.73 | 15 | 6.4 | 13.96 | 17.52 | 23.03 | 30.36 | 11.39 | 13.73 | −1.62 |
| Ehd2 | 13.39 | 1.77 | 6.08 | 1.28 | 2.88 | 21.98 | 7.27 | 2.65 | 3.67 | 2.46 | 3.1 | −1.62 |
| Anxa11 | 6.9 | 1.96 | 2.14 | 1 | 3.69 | 14.21 | 12.4 | 5.73 | 6.93 | 7.96 | 7.33 | −1.62 |
| Flnb | 18.28 | 5.82 | 6.12 | 4.47 | 7.41 | 11.84 | 8.24 | 5.2 | 5.69 | 6.3 | 4.99 | −1.61 |
| Fam198b | 5.35 | 0.45 | 1.5 | 0.65 | 0.5 | 1.68 | 1.8 | 0.62 | 0.36 | 0.4 | 0.42 | −1.61 |
| Aldh1a2 | 420.19 | 104.79 | 147.51 | 126.72 | 137.45 | 175.71 | 174.37 | 559.18 | 415.9 | 343.11 | 284.41 | −1.61 |
| Galnt16 | 4.04 | 0.52 | 0.83 | 0.48 | 0.88 | 3.2 | 1.37 | 0.43 | 0.66 | 1.8 | 1.72 | −1.61 |
| Ccl11 | 2.36 | 0.18 | 0.21 | 0 | 0.18 | 4.1 | 1.22 | 0.05 | 0 | 0 | 0 | −1.60 |
| Rab32 | 6.99 | 0.54 | 2.03 | 1.23 | 0.88 | 5.9 | 8.69 | 0 | 0 | 0 | 0 | −1.60 |
| Syt5 | 6.26 | 1.59 | 1.7 | 1.08 | 1.21 | 2.26 | 1.32 | 0.75 | 1.07 | 0.43 | 0.7 | −1.60 |
| Pcdh7 | 4.1 | 0.11 | 0.68 | 0.68 | 0.23 | 0.65 | 1.27 | 2.65 | 0.66 | 1.65 | 0.24 | −1.60 |
| Bcas3os2 | 2.71 | 0.51 | 0.39 | 0.06 | 0.34 | 0.65 | 0.25 | 1.08 | 2.77 | 2.94 | 4.2 | −1.60 |
| Cblc | 2.24 | 0.12 | 0.14 | 0 | 0.27 | 3.31 | 3.86 | 0.18 | 0.18 | 0.46 | 0.47 | −1.60 |
| 6720468P15Rik | 3.25 | 0.34 | 0.42 | 0.39 | 0.62 | 1.7 | 3.53 | 0 | 0.07 | 0 | 0.12 | −1.60 |
| Batf3 | 13.64 | 6.04 | 4.75 | 2.94 | 8.56 | 14.62 | 10.69 | 5.61 | 4.03 | 8.7 | 7.77 | −1.60 |
| Cgn | 4.01 | 0.62 | 0.68 | 0.64 | 0.63 | 3.77 | 1.86 | 0.37 | 0.29 | 0.43 | 0.56 | −1.59 |
| Tpm1 | 248.3 | 69.68 | 86.18 | 77.42 | 84.49 | 220.69 | 169.74 | 85.76 | 131.27 | 134.91 | 121.93 | −1.59 |
| Mboat1 | 7.72 | 1.72 | 2.28 | 1.53 | 2.09 | 6.72 | 5.4 | 2.99 | 3.15 | 3.86 | 2.85 | −1.59 |
| Lrfn2 | 3.81 | 0.52 | 0.68 | 0.53 | 0.36 | 0.82 | 0.58 | 0.33 | 0.37 | 0.15 | 0.19 | −1.58 |
| Prdm6 | 3.48 | 0.16 | 0.6 | 0.39 | 0.13 | 0.03 | 0.17 | 0.13 | 0.46 | 1.15 | 1.59 | −1.58 |
| Mir682 | 137.62 | 144.52 | 56.17 | 34.79 | 146.25 | 279.9 | 118.45 | 55.9 | 395.17 | 314.23 | 0 | −1.58 |
| Angptl4 | 19.29 | 6.55 | 6.93 | 4.7 | 7.36 | 7.61 | 20.32 | 5.27 | 3.84 | 2.7 | 3.13 | −1.57 |
| Zeb2 | 8.97 | 0.8 | 2.26 | 2.44 | 0.86 | 2.93 | 7.41 | 3.03 | 1.42 | 0.66 | 0.53 | −1.57 |
| Nab2 | 42.37 | 17.13 | 16.05 | 11.27 | 24.64 | 43.17 | 26.76 | 65.88 | 42.47 | 75.3 | 52.91 | −1.56 |
| Lasp1 | 54.56 | 20.49 | 20.86 | 14.72 | 23.91 | 53.21 | 34.7 | 35.23 | 43.7 | 33.74 | 31.71 | −1.56 |
| Dcbld1 | 13.65 | 2.5 | 4.21 | 3.74 | 3.36 | 6.24 | 5.71 | 6.41 | 5.63 | 6.6 | 6.52 | −1.56 |
| Cdc42ep2 | 6.45 | 1.13 | 2.48 | 0.58 | 1.51 | 14.95 | 7.24 | 12.04 | 4.52 | 6.29 | 5.92 | −1.56 |
| Cdh9 | 2.88 | 0.03 | 0.3 | 0.34 | 0.06 | 0 | 0 | 0 | 0 | 0 | 0 | −1.56 |
| Tns3 | 15.07 | 4.04 | 4.88 | 4.07 | 3.62 | 8.25 | 10 | 4.17 | 5.36 | 9.88 | 8.85 | −1.55 |
| Cald1 | 48.19 | 6.53 | 14.72 | 16.84 | 8.17 | 17.34 | 37.23 | 20.5 | 11.36 | 10.82 | 11.23 | −1.55 |
| S100a16 | 47.38 | 12.53 | 23.1 | 8.02 | 15.95 | 129.61 | 95.8 | 14.65 | 18.82 | 42.83 | 18.43 | −1.55 |
| Fgd5 | 5.65 | 0.52 | 2.15 | 0.41 | 0.7 | 5.94 | 3.13 | 2.21 | 1.5 | 1.34 | 2.41 | −1.54 |
| Rcn1 | 108.86 | 20.29 | 36.68 | 36.67 | 24.88 | 49.15 | 55.85 | 111.4 | 96.47 | 95.19 | 79.68 | −1.54 |
| Emilin3 | 6.27 | 1.79 | 1.79 | 1.2 | 2.15 | 2.75 | 1.16 | 0.19 | 0.51 | 0.1 | 0.3 | −1.54 |
| 2610027K06Rik | 2.27 | 0 | 0.2 | 0.05 | 0.11 | 1.28 | 0.46 | 0.86 | 0.77 | 0.24 | 1.22 | −1.54 |
| Lrrc4b | 2.8 | 0.21 | 0.33 | 0.29 | 0.52 | 1.8 | 1.23 | 1.97 | 2 | 0.9 | 0.6 | −1.54 |
| BC021891 | 2.64 | 0.15 | 0.32 | 0.19 | 0.35 | 0.96 | 1.58 | 3.9 | 2.94 | 4.86 | 5.14 | −1.54 |
| Eps8l2 | 4.22 | 0.91 | 1.02 | 0.58 | 0.98 | 9.85 | 18.03 | 0.68 | 1.38 | 2.42 | 0.95 | −1.54 |
| Cck | 3.6 | 0 | 0.73 | 0.45 | 0 | 1.98 | 2.87 | 0.27 | 0.16 | 0.34 | 0.28 | −1.53 |
| Fam129b | 22.93 | 7.39 | 8.94 | 5.61 | 9.88 | 29.61 | 13.35 | 23.3 | 23.98 | 20.33 | 16.78 | −1.53 |
| Nek6 | 37.12 | 10.23 | 13.87 | 10.5 | 13.49 | 18.83 | 10.91 | 25.15 | 24.9 | 15.59 | 17.32 | −1.53 |
| Crh | 2.31 | 0 | 0.27 | 0.02 | 0 | 0.07 | 0.1 | 0 | 0 | 0 | 0 | −1.53 |
| Epb4.1l1 | 8.33 | 2.15 | 2.55 | 1.91 | 3.51 | 6.41 | 6.55 | 6.56 | 9.63 | 5.96 | 6.31 | −1.53 |
| Ccdc42 | 4.94 | 0.53 | 1.01 | 1.11 | 0.47 | 2.59 | 1.28 | 0.43 | 0.75 | 0.36 | 0 | −1.53 |
| Slc38a4 | 57.97 | 8.83 | 19.81 | 19.1 | 13.44 | 18.02 | 25.72 | 51.49 | 34.05 | 18.91 | 24.25 | −1.53 |
| Sfta2 | 2.99 | 0.76 | 0.69 | 0.09 | 0 | 2.7 | 3 | 0 | 0 | 0 | 0 | −1.52 |
| Cyp46a1 | 5.78 | 1.88 | 1.79 | 0.94 | 1.91 | 5.12 | 3.92 | 6.79 | 8.88 | 13.69 | 8.54 | −1.52 |
| Wscd2 | 3.24 | 0.67 | 0.66 | 0.3 | 0.59 | 1 | 0.35 | 1.02 | 1.42 | 0.14 | 0.37 | −1.52 |
| Leprel2 | 40.94 | 17.67 | 16.53 | 10.77 | 23.68 | 39.48 | 31.99 | 19.82 | 19.25 | 20.74 | 15.7 | −1.52 |
| Klhl4 | 6.03 | 0.13 | 2.4 | 0.53 | 0.35 | 3.19 | 0.82 | 0 | 0 | 0 | 0 | −1.51 |
| Hmha1 | 13.97 | 4.8 | 5.96 | 2.54 | 5.5 | 9.75 | 8.12 | 0.49 | 0.22 | 0.25 | 0.12 | −1.51 |
| 4930523C07Rik | 2.92 | 0.21 | 0.49 | 0.26 | 0.74 | 2.13 | 3.59 | 0.01 | 0.16 | 0.3 | 0.28 | −1.51 |
| Grm3 | 2.15 | 0.05 | 0.19 | 0.02 | 0.3 | 0.13 | 0.02 | 0 | 0 | 0 | 0 | −1.51 |
| Tspan11 | 4.77 | 1.28 | 1.21 | 0.84 | 1.51 | 2.56 | 1.87 | 3.5 | 2.89 | 2.67 | 1.77 | −1.51 |
| Fgf12 | 2.12 | 0 | 0.12 | 0.07 | 0.05 | 0.27 | 0.74 | 0 | 0 | 0 | 0 | −1.51 |
| Gpr39 | 2.09 | 0 | 0.15 | 0.02 | 0.25 | 3.55 | 1.44 | 0 | 0.07 | 0.16 | 0 | −1.51 |
| L1td1 | 2.33 | 0.14 | 0.28 | 0.06 | 0.11 | 0.19 | 0 | 0 | 0 | 0 | 0 | −1.51 |
| Gprc5b | 9.1 | 2.23 | 3.12 | 1.98 | 2.9 | 5.09 | 4.4 | 0.24 | 0.41 | 0.91 | 0.52 | −1.51 |
| Kdelr3 | 17.31 | 8.81 | 6.32 | 4.58 | 12.13 | 27.27 | 24.77 | 84.59 | 96.52 | 40.93 | 36.68 | −1.51 |
| Fbn2 | 41.58 | 13.95 | 14.22 | 13.82 | 21.28 | 16.35 | 15.06 | 13.59 | 14.14 | 12.11 | 12.09 | −1.50 |
| Lrrc4 | 4.19 | 0.49 | 0.81 | 0.86 | 0.68 | 0.93 | 0.65 | 0.1 | 0.07 | 0.12 | 0.04 | −1.50 |
| Myh10 | 83.29 | 28.64 | 29.52 | 28.15 | 31.69 | 40.96 | 29.9 | 32.73 | 32.54 | 29.12 | 27 | −1.50 |
| Thbs3 | 19.25 | 7.47 | 7.55 | 4.79 | 10.64 | 10.53 | 6.17 | 2.81 | 3.43 | 4.12 | 3.66 | −1.50 |
| Slc1a3 | 9.57 | 2.63 | 2.85 | 2.64 | 6.48 | 11.92 | 15.39 | 4.78 | 2.69 | 2.33 | 2.42 | −1.50 |
| Cmpk2 | 5.05 | 0.9 | 1.27 | 1.02 | 0.86 | 1.77 | 2.77 | 0.3 | 0.32 | 0.11 | 0.46 | −1.50 |
| Akr1b7 | 3.23 | 0.07 | 0.63 | 0.37 | 0.82 | 5.2 | 3.07 | 2.39 | 3.62 | 0.96 | 1.56 | −1.50 |
| Myof | 3.9 | 0.68 | 0.82 | 0.66 | 0.88 | 5.32 | 8.04 | 5.14 | 6.16 | 9.69 | 6.98 | −1.49 |
| Ednrb | 8.49 | 0.3 | 2.99 | 1.75 | 0.53 | 5.68 | 9.06 | 3.65 | 1.53 | 0.78 | 0.68 | −1.49 |
| Ano1 | 4.05 | 0.96 | 1.03 | 0.56 | 2.19 | 7.34 | 4.82 | 2.02 | 2.21 | 0.47 | 1.12 | −1.49 |
| Slc18b1 | 7.1 | 1.34 | 1.86 | 1.9 | 2.15 | 5.93 | 7.3 | 3.09 | 2.48 | 2.69 | 2.74 | −1.49 |
| Lgi2 | 3.94 | 0.85 | 0.81 | 0.71 | 1.71 | 3.02 | 1.8 | 5.63 | 5.53 | 1.36 | 2.13 | −1.49 |
| Kctd12b | 3.98 | 0.3 | 1.03 | 0.52 | 0.15 | 2.17 | 4.97 | 0.19 | 0.14 | 0.11 | 0.06 | −1.49 |
| Tbkbp1 | 15.16 | 5.37 | 6.58 | 2.96 | 6.45 | 11.99 | 3.93 | 9.39 | 7.59 | 5.12 | 5.57 | −1.49 |
| Prrx1 | 15.06 | 1.74 | 3.92 | 5.57 | 1.45 | 1.71 | 0.83 | 8.28 | 2.29 | 0.47 | 1.7 | −1.48 |
| Chl1 | 3.58 | 0.31 | 0.7 | 0.58 | 0.21 | 0.02 | 0.03 | 0 | 0 | 0 | 0 | −1.48 |
| Fhl2 | 23.28 | 2.72 | 5.16 | 10.23 | 5.89 | 37.05 | 79.29 | 61.71 | 31.83 | 63.42 | 41.96 | −1.48 |
| D630003M21Rik | 2.75 | 0.41 | 0.51 | 0.18 | 0.48 | 2.28 | 0.71 | 0.21 | 0.19 | 0.52 | 0.15 | −1.48 |
| Sult5a1 | 8.17 | 1.77 | 2.71 | 1.88 | 1.54 | 8.39 | 9.55 | 0.88 | 1.32 | 0.17 | 0 | −1.48 |
| Limch1 | 10.02 | 2.17 | 3.45 | 2.47 | 2.7 | 7.43 | 11.9 | 7.34 | 6.83 | 6.85 | 6.76 | −1.48 |
| Mir6395 | 65.84 | 0 | 23.89 | 22.19 | 124.39 | 0 | 0 | 0 | 0 | 0 | 0 | −1.48 |
| Slc10a3-ubl4 | 3.94 | 0.56 | 1.42 | 0.14 | 2.81 | 3.3 | 0 | 0.69 | 0 | 0.51 | 0 | −1.47 |
| Tbx1 | 3.27 | 0 | 0.92 | 0.16 | 0 | 0.33 | 0.14 | 1.33 | 1.44 | 0.42 | 1.55 | −1.47 |
| March11 | 2.41 | 0.06 | 0.38 | 0.08 | 0.06 | 0.11 | 0.14 | 0 | 0 | 0 | 0 | −1.47 |
| Fbn1 | 9.49 | 3.24 | 3.01 | 2.57 | 4.15 | 8.58 | 10.97 | 4.05 | 3.64 | 4.41 | 3.2 | −1.47 |
| Atoh8 | 10.02 | 2.28 | 4.09 | 1.88 | 3.76 | 9.95 | 5.27 | 3.48 | 7.14 | 7.95 | 7.41 | −1.47 |
| Laptm4b | 71.83 | 17.63 | 26.25 | 24.47 | 21.19 | 47.49 | 50.73 | 99.96 | 65.9 | 63.42 | 51.71 | −1.47 |
| St8sia1 | 2.92 | 0.22 | 0.49 | 0.35 | 0.34 | 1.18 | 1.42 | 0.6 | 0.38 | 0.25 | 0.39 | −1.46 |
| Trim10 | 1.76 | 0 | 0 | 0 | 0.08 | 0.15 | 0 | 0 | 0 | 0 | 0 | −1.46 |
| Tmsb4x | 1184.92 | 196.53 | 465.81 | 391.94 | 174.91 | 1341.94 | 2168.94 | 1433.53 | 1181.3 | 1301.31 | 1381.69 | −1.46 |
| St8sia2 | 18.52 | 5.83 | 7.29 | 4.89 | 6.73 | 6.81 | 3.8 | 1.18 | 3.42 | 5.31 | 4.13 | −1.46 |
| Hpn | 9.53 | 3.09 | 3.47 | 2.18 | 2.22 | 11.35 | 25.24 | 5.61 | 10.19 | 2.33 | 2.65 | −1.46 |
| Bhlhe22 | 1.93 | 0 | 0.08 | 0.05 | 0 | 0.3 | 0.73 | 0 | 0 | 0 | 0 | −1.46 |
| Eno2 | 5.28 | 1.61 | 1.65 | 0.92 | 2.11 | 6.95 | 5.19 | 56.07 | 33.43 | 17.02 | 17.95 | −1.46 |
| Phactr2 | 11.04 | 2 | 3.6 | 3.18 | 2.7 | 4.59 | 8.16 | 4.48 | 3.23 | 2.68 | 2.68 | −1.46 |
| Akr1c19 | 2.82 | 0.52 | 0.34 | 0.45 | 0.9 | 5.15 | 15.16 | 0.09 | 0 | 0.4 | 0.26 | −1.45 |
| Gulp1 | 11.61 | 0.66 | 3.34 | 3.88 | 0.67 | 0.91 | 9.28 | 2.51 | 0.31 | 0.48 | 0.44 | −1.45 |
| Plau | 6.53 | 0.47 | 2.58 | 0.93 | 0.69 | 13.24 | 112.12 | 2.37 | 0.99 | 2.3 | 2.48 | −1.45 |
| Boc | 41.21 | 14.26 | 16.24 | 12.69 | 15.92 | 18.21 | 17.94 | 9.74 | 9.39 | 19.04 | 15.73 | −1.45 |
| Ripk4 | 3.24 | 0.05 | 0.66 | 0.46 | 0.26 | 2.93 | 2.83 | 0.19 | 0.07 | 0.27 | 0.12 | −1.44 |
| Bmp1 | 72.61 | 36.29 | 32.71 | 19.53 | 47.61 | 53.51 | 37.2 | 20.38 | 27.13 | 35.01 | 38.17 | −1.44 |
| Nid2 | 19.07 | 5.41 | 8.3 | 4.49 | 7.5 | 27.44 | 17 | 120.54 | 175.24 | 155.63 | 109.69 | −1.44 |
| Serpina3g | 2.27 | 0.04 | 0.27 | 0.14 | 0.26 | 1.24 | 14.51 | 20.85 | 16.96 | 15.34 | 12.48 | −1.44 |
| Srgap3 | 5.13 | 1.3 | 1.51 | 1.02 | 1.9 | 3 | 2.39 | 1.44 | 1.55 | 0.74 | 1.05 | −1.44 |
| Spint2 | 88.16 | 48.2 | 36.35 | 27.54 | 42.15 | 120.13 | 148.36 | 34.84 | 78.5 | 119.06 | 96.64 | −1.44 |
| Ptgr1 | 22.34 | 5.69 | 7.97 | 7.29 | 8.67 | 32.34 | 15.33 | 18.61 | 17.6 | 22.88 | 15.62 | −1.44 |
| Grik2 | 2.15 | 0.1 | 0.18 | 0.15 | 0.05 | 0.39 | 0.11 | 0 | 0 | 0 | 0 | −1.44 |
| Wisp1 | 2.36 | 0.08 | 0.3 | 0.19 | 0.08 | 1.94 | 3.19 | 4.27 | 2.45 | 2.82 | 1.55 | −1.43 |
| Csf1 | 5.03 | 0.6 | 1.44 | 1.03 | 1.48 | 5.15 | 3.68 | 5.95 | 7.75 | 14.05 | 11.99 | −1.43 |
| Adamts9 | 27.15 | 6.79 | 9.9 | 8.99 | 8.04 | 9.39 | 9.5 | 11.72 | 13.39 | 5.69 | 6.84 | −1.43 |
| Dkk2 | 3.32 | 0.04 | 0.72 | 0.49 | 0.38 | 1.21 | 1.92 | 12.6 | 4.11 | 1.62 | 1.48 | −1.43 |
| Tmeff2 | 3.24 | 0.44 | 0.69 | 0.47 | 0.5 | 1.11 | 0.41 | 0 | 0 | 0 | 0.04 | −1.42 |
| Apbb2 | 8.39 | 1.83 | 2.74 | 2.26 | 2.33 | 4.77 | 6.48 | 7.99 | 7.64 | 7.44 | 7.31 | −1.42 |
| Sema3a | 2.97 | 0.14 | 0.67 | 0.29 | 0.32 | 2.44 | 5.24 | 2.76 | 0.3 | 0.07 | 0.04 | −1.42 |
| Cygb | 3.57 | 0.61 | 0.84 | 0.57 | 1.42 | 6.67 | 20.12 | 2.07 | 2.33 | 6.28 | 4.74 | −1.42 |
| Fam19a1 | 2.27 | 0.08 | 0.27 | 0.17 | 0.33 | 0.61 | 1.94 | 0.54 | 0.1 | 0.14 | 0.04 | −1.42 |
| Tec | 7.33 | 1.31 | 2.64 | 1.58 | 1.31 | 3 | 4.58 | 1.42 | 1.54 | 4.39 | 4.23 | −1.42 |
| Ppp1r13b | 9.84 | 3.23 | 4.16 | 1.94 | 4.27 | 13.1 | 6.75 | 2.76 | 2.99 | 4.2 | 3.59 | −1.42 |
| Tcf7 | 27.61 | 7.71 | 11.09 | 8.3 | 11.23 | 13.1 | 14.33 | 20.98 | 27.38 | 27.42 | 27.5 | −1.42 |
| Plcl1 | 2.33 | 0.17 | 0.36 | 0.13 | 0.21 | 1.17 | 1.7 | 0.54 | 0.17 | 0.07 | 0.24 | −1.42 |
| Scn8a | 3.09 | 0.28 | 0.72 | 0.34 | 0.58 | 0.96 | 0.63 | 0.25 | 0.41 | 0.52 | 0.86 | −1.42 |
| Tns1 | 9.14 | 2.62 | 3.31 | 2.28 | 2.23 | 5.68 | 10.79 | 4.26 | 6.55 | 4.1 | 4.24 | −1.42 |
| Krt19 | 2.86 | 0.53 | 0.46 | 0.43 | 1.53 | 167.04 | 35.96 | 83.6 | 87.09 | 120.95 | 59.63 | −1.42 |
| Epb4.1l2 | 37.03 | 9.02 | 14.02 | 12.47 | 10.9 | 31.93 | 27.72 | 25.89 | 19.33 | 17.46 | 16.07 | −1.42 |
| Hoxd13 | 2.79 | 0.13 | 0.31 | 0.53 | 0 | 0.1 | 0.12 | 15.2 | 1.17 | 0.07 | 0.48 | −1.42 |
| Col9a3 | 4.9 | 0.95 | 1.5 | 0.93 | 1.38 | 1.14 | 0.95 | 0.91 | 0.77 | 0.63 | 0.41 | −1.41 |
| Kank1 | 8.04 | 2.14 | 2.39 | 2.41 | 2.85 | 7.74 | 6.99 | 4.09 | 5.46 | 7.53 | 8 | −1.41 |
| Anpep | 9.82 | 3.41 | 3.64 | 2.5 | 5.16 | 17.94 | 29.34 | 8.04 | 11.78 | 6.95 | 7.66 | −1.41 |
| Ly6h | 12.77 | 4.3 | 5.79 | 2.57 | 3.02 | 8.33 | 3.73 | 0.13 | 0.23 | 0.12 | 0.39 | −1.41 |
| Lama2 | 2.4 | 0.13 | 0.32 | 0.24 | 0.42 | 2.56 | 3.29 | 0.64 | 0.44 | 0.24 | 0.11 | −1.41 |
| Actg2 | 3.26 | 0.07 | 0.59 | 0.62 | 0.14 | 1.67 | 0.58 | 0 | 0 | 0 | 0 | −1.41 |
| Abcg5 | 6.95 | 1.64 | 2.26 | 1.74 | 1.4 | 2.13 | 1.01 | 0.13 | 0.23 | 0.04 | 0.2 | −1.41 |
| Ntn3 | 5.05 | 1.78 | 1.61 | 0.96 | 3.41 | 3.99 | 1.58 | 1 | 1.44 | 1.32 | 1.1 | −1.40 |
| Atp1b1 | 8.24 | 1.38 | 2.13 | 2.85 | 3.04 | 60.36 | 572.05 | 36.67 | 31.4 | 28.69 | 21.45 | −1.40 |
| Lypd6 | 3.44 | 0.3 | 0.74 | 0.62 | 0.64 | 3.55 | 1.4 | 2.39 | 2.34 | 1.67 | 2.28 | −1.40 |
| Map7 | 4.72 | 0.8 | 1.33 | 1 | 0.95 | 3.16 | 8.15 | 0.33 | 0.49 | 0.66 | 0.85 | −1.40 |
| Ncald | 9.49 | 3.13 | 2.87 | 3.08 | 2.12 | 4.76 | 5.14 | 3.67 | 3.18 | 1.32 | 1.35 | −1.40 |
| Rassf10 | 5.28 | 0.86 | 1.44 | 1.33 | 1.04 | 1.35 | 2.52 | 0.61 | 0.84 | 0.48 | 0.25 | −1.40 |
| Arhgef9 | 7.16 | 1.95 | 2.24 | 1.96 | 1.76 | 3.24 | 3.96 | 0.6 | 0.25 | 0.85 | 0.49 | −1.40 |
| Arsb | 11.08 | 2.55 | 3.74 | 3.44 | 4.45 | 7.15 | 11.31 | 1.88 | 2.04 | 3.9 | 2.66 | −1.40 |
| Pou6f1 | 5.52 | 1.37 | 1.85 | 1.12 | 2.83 | 4.32 | 5.66 | 2.38 | 3.28 | 3.07 | 3.21 | −1.39 |
| Pdzrn4 | 2.45 | 0.1 | 0.36 | 0.27 | 0.24 | 0.23 | 0.72 | 0.91 | 0.17 | 0 | 0.04 | −1.39 |
| Tmem45a | 7.19 | 0.92 | 1.67 | 2.58 | 0.97 | 3.72 | 6.39 | 3.48 | 0.57 | 0.3 | 0 | −1.39 |
| Sdc2 | 21.71 | 5.41 | 7.17 | 8.18 | 5.85 | 17.28 | 34.02 | 0.11 | 1.06 | 5.13 | 3.24 | −1.39 |
| Apcdd1 | 7.6 | 2.04 | 2.62 | 1.96 | 3.95 | 18.73 | 9.67 | 1.1 | 1.73 | 1.99 | 1.68 | −1.39 |
| 1010001N08Rik | 2.63 | 0 | 0.31 | 0.47 | 0 | 0.9 | 1.23 | 0.1 | 0 | 1.13 | 0 | −1.38 |
| Tgfb3 | 23.23 | 10.4 | 9.34 | 7.22 | 12.06 | 17.03 | 14.06 | 13.24 | 8.83 | 8.34 | 4.97 | −1.38 |
| Ccdc102a | 21.87 | 9.76 | 9.57 | 6.02 | 11.39 | 18.79 | 7.12 | 5.81 | 8.21 | 8.34 | 6.87 | −1.38 |
| LOC102636514 | 2.04 | 0 | 0.34 | 0 | 0 | 0 | 0 | 0 | 0 | 0 | 0 | −1.38 |
| Adcy2 | 16.29 | 6.05 | 6.56 | 4.75 | 6.8 | 6.73 | 3.46 | 6.33 | 5.62 | 3.31 | 3.74 | −1.38 |
| Pbx1 | 118.13 | 33.51 | 41.61 | 48.18 | 33.49 | 45.09 | 46.47 | 10.28 | 9.87 | 10.9 | 10.76 | −1.38 |
| Clip2 | 20.85 | 9.43 | 8.7 | 6.19 | 8.55 | 10.03 | 6.18 | 13.17 | 12.34 | 11.57 | 10.22 | −1.37 |
| Fkbp10 | 86.72 | 41.47 | 41.68 | 24.19 | 58.75 | 98.13 | 74.24 | 69.9 | 86.5 | 125.99 | 86.82 | −1.37 |
| Selm | 56.19 | 28.14 | 25.35 | 16.96 | 33.47 | 87.97 | 46.03 | 109.75 | 103.51 | 120.61 | 84.36 | −1.37 |
| Enpep | 2.83 | 0.31 | 0.53 | 0.44 | 1.13 | 6.74 | 18.36 | 2.95 | 1.61 | 5.56 | 5.82 | −1.37 |
| Hebp2 | 5.64 | 1.17 | 1.49 | 1.66 | 1.64 | 5.53 | 2.79 | 3.24 | 3.23 | 1.52 | 1.6 | −1.37 |
| Efna3 | 12.51 | 5.94 | 5.32 | 3.17 | 5.32 | 6.91 | 1.37 | 10.77 | 5.93 | 3.35 | 3.87 | −1.37 |
| Tgfbr3 | 4.46 | 1.04 | 1.12 | 1.12 | 1.02 | 2.35 | 3.57 | 4.65 | 3.4 | 1.44 | 1.93 | −1.36 |
| Adamts7 | 9.89 | 5.58 | 4.16 | 2.3 | 5.14 | 6.2 | 3.85 | 1.27 | 1.62 | 1.2 | 1.83 | −1.36 |
| Rbms3 | 13.39 | 1.67 | 3.95 | 5.23 | 1.49 | 4.66 | 13.34 | 10.92 | 4.84 | 4.71 | 4.88 | −1.36 |
| Cdh10 | 2.1 | 0 | 0.28 | 0.13 | 0.07 | 0.26 | 0.12 | 0 | 0 | 0.03 | 0 | −1.36 |
| Tnnt1 | 10.76 | 6.41 | 4.42 | 2.73 | 4.32 | 10.44 | 9.17 | 2.68 | 2.39 | 0.63 | 1.55 | −1.36 |
| Pkp2 | 5 | 1.31 | 1.23 | 1.44 | 1.3 | 2.96 | 3.72 | 1.59 | 3.1 | 2.64 | 4.41 | −1.36 |
| Pwwp2b | 3.92 | 0.83 | 1.21 | 0.62 | 1.24 | 6.5 | 3.3 | 6.76 | 5.8 | 14.31 | 10.42 | −1.36 |
| Fgf11 | 5.73 | 1.32 | 1.88 | 1.36 | 2.1 | 4.47 | 7.49 | 14.37 | 9.66 | 14.8 | 8.59 | −1.36 |
| Syt13 | 3.9 | 1.08 | 1.03 | 0.79 | 0.9 | 2.2 | 1.17 | 9.68 | 9.87 | 2.28 | 1.38 | −1.36 |
| lqgap2 | 5.72 | 1.08 | 1.61 | 1.63 | 1.16 | 2.38 | 3.84 | 4.04 | 3.24 | 4.03 | 3.86 | −1.36 |
| Lrrtm1 | 1.88 | 0.03 | 0.17 | 0.08 | 0.4 | 1.09 | 0.65 | 0 | 0 | 0.04 | 0 | −1.36 |
| Nlgn3 | 2.76 | 0.46 | 0.62 | 0.32 | 0.87 | 1.64 | 0.5 | 0.15 | 0.24 | 0.29 | 0.04 | −1.35 |
| Arhgap31 | 4.29 | 0.77 | 1.5 | 0.64 | 0.48 | 3.67 | 2.61 | 0.59 | 0.87 | 0.82 | 0.33 | −1.35 |
| Chrnb1 | 3.65 | 1.14 | 0.81 | 0.83 | 1.28 | 4.39 | 3.86 | 6.3 | 4.49 | 1.59 | 3.01 | −1.35 |
| Bmp5 | 2.41 | 0.07 | 0.4 | 0.27 | 0.03 | 0.19 | 0.1 | 0.06 | 0 | 0.04 | 0 | −1.35 |
| Gm6213 | 3.85 | 0.71 | 1.24 | 0.57 | 0.72 | 4.45 | 0.26 | 0 | 0 | 0 | 0 | −1.35 |
| Tyro3 | 13.21 | 6.29 | 5.21 | 3.96 | 7.31 | 10.25 | 6.19 | 9.73 | 9.4 | 6.77 | 8.13 | −1.35 |
| Nr2f1 | 108.25 | 26.57 | 42.68 | 41.29 | 23.05 | 23.91 | 15.35 | 0.04 | 0 | 0.86 | 0 | −1.35 |
| Arhgap29 | 8.07 | 0.69 | 3.24 | 1.91 | 1.24 | 6.78 | 11.05 | 6.49 | 3.56 | 5.55 | 5.96 | −1.34 |
| Tmem238 | 5.24 | 2.36 | 1.87 | 1.05 | 3.39 | 5.96 | 4.23 | 2.26 | 1.67 | 3.94 | 2.89 | −1.34 |
| C430049B03Rik | 17.16 | 2.56 | 6.55 | 5.78 | 1.64 | 4.34 | 6.31 | 34.75 | 13.99 | 7.32 | 6.55 | −1.34 |
| Clstn2 | 8.21 | 2.04 | 2.9 | 2.37 | 2.19 | 1.56 | 1.67 | 0.12 | 0.68 | 0.99 | 1.05 | −1.34 |
| Procr | 3.33 | 0.35 | 1.06 | 0.36 | 0.18 | 11.09 | 4.01 | 4.38 | 3.87 | 2.05 | 2.09 | −1.34 |
| Socs2 | 3.38 | 0.04 | 0.97 | 0.49 | 0.29 | 1.84 | 42.71 | 8.28 | 3.87 | 2.13 | 5.45 | −1.34 |
| Megf6 | 5.38 | 2.48 | 1.91 | 1.13 | 3.62 | 2.64 | 0.99 | 1.13 | 0.66 | 0.14 | 0.53 | −1.34 |
| Leprel1 | 2 | 0.04 | 0.29 | 0.08 | 0.49 | 2.54 | 5.48 | 1.57 | 2.31 | 1.84 | 1.6 | −1.34 |
| Msrb3 | 6.54 | 1.2 | 2.26 | 1.7 | 1.26 | 6.66 | 6.73 | 15.34 | 9.03 | 5.33 | 3.65 | −1.34 |
| Tbc1d9 | 6.59 | 1.77 | 2.3 | 1.71 | 2.06 | 2.45 | 3.14 | 3.78 | 4.71 | 6.55 | 4.79 | −1.34 |
| Nrcam | 2.28 | 0.14 | 0.33 | 0.27 | 0.07 | 0.54 | 0.92 | 0.49 | 0.56 | 0.09 | 0.25 | −1.34 |
| Irf2bpl | 16.75 | 4.54 | 6.04 | 6.04 | 5.19 | 8.16 | 14.71 | 15.13 | 8.32 | 13.58 | 9.65 | −1.33 |
| Al661453 | 2 | 0.35 | 0.27 | 0.11 | 0.19 | 2.74 | 3.52 | 0.5 | 0.34 | 0.47 | 0.23 | −1.33 |
| Irx3 | 3.26 | 0.43 | 1.07 | 0.31 | 0.62 | 7.14 | 3.82 | 0.91 | 1.58 | 2.99 | 3.05 | −1.33 |
| Cldn11 | 7.54 | 0.78 | 2.22 | 2.56 | 2 | 24.37 | 8.08 | 0.27 | 0.05 | 0.5 | 0 | −1.33 |
| Prickle2 | 5.32 | 0.91 | 1.53 | 1.49 | 1.03 | 1.8 | 2.61 | 1.28 | 1.17 | 0.59 | 0.69 | −1.33 |
| Wls | 38.57 | 9.95 | 14.64 | 14.96 | 12.79 | 29.29 | 62.76 | 79.59 | 82.25 | 86.36 | 95.98 | −1.32 |
| Tubb2b | 69.1 | 36.49 | 30.19 | 23.8 | 36.07 | 31.83 | 23.83 | 49.24 | 51.57 | 51.76 | 67.28 | −1.32 |
| Vim | 461.94 | 157.61 | 205.52 | 162.31 | 153.45 | 393.47 | 238.19 | 651.55 | 677.41 | 517.38 | 614.35 | −1.32 |
| Nedd4l | 6.28 | 1.87 | 2.13 | 1.69 | 1.69 | 5.83 | 5.03 | 8.02 | 8.34 | 7.86 | 8.47 | −1.32 |
| Lhfp | 41.77 | 10.34 | 15.57 | 16.64 | 12.15 | 35.09 | 92.23 | 2.48 | 3.63 | 10.05 | 10.26 | −1.32 |
| Faah | 3.05 | 0.91 | 0.73 | 0.51 | 1.03 | 3.58 | 1.84 | 0.12 | 0.07 | 0.12 | 0 | −1.32 |
| Stfa2 | 1.5 | 0 | 0 | 0 | 0 | 1.36 | 11.19 | 0 | 0 | 0 | 0 | −1.32 |
| Smarca1 | 8.66 | 0.64 | 2.63 | 3.1 | 1.16 | 2.56 | 4.92 | 2.96 | 1.4 | 0.15 | 0.29 | −1.32 |
| Jph2 | 3.23 | 0.51 | 0.79 | 0.61 | 0.81 | 2.13 | 1.22 | 0.06 | 0.04 | 0.06 | 0 | −1.32 |
| Sertad4 | 14.51 | 3.72 | 5.08 | 5.39 | 4.87 | 11.25 | 20.3 | 2.62 | 3.11 | 1.5 | 2.69 | −1.31 |
| Dpp4 | 1.76 | 0.06 | 0.16 | 0.06 | 0.51 | 3.1 | 6.39 | 2.02 | 0.87 | 0.02 | 0.13 | −1.31 |
| Gng8 | 8.07 | 2.89 | 3.33 | 1.97 | 3.04 | 5.29 | 4.32 | 7.15 | 5.6 | 1.4 | 3.23 | −1.31 |
| 1810011O10Rik | 70.34 | 10.35 | 28.1 | 27.38 | 8.75 | 35.34 | 65.82 | 38.08 | 17.31 | 32.79 | 33.1 | −1.31 |
| Irs1 | 3.85 | 0.77 | 1.03 | 0.88 | 1.2 | 2.61 | 5.43 | 2.26 | 1.61 | 2.19 | 2.2 | −1.31 |
| Calcr | 1.74 | 0.02 | 0.15 | 0.06 | 0 | 0 | 0.2 | 0.04 | 0.02 | 0 | 0 | −1.31 |
| Rarb | 29.11 | 6.62 | 10.57 | 11.73 | 7.61 | 16.14 | 13.49 | 1.24 | 0.38 | 0.27 | 0.38 | −1.31 |
| Mid1ip1 | 12.03 | 3.27 | 5.26 | 3.26 | 5.24 | 15.41 | 12.85 | 7.02 | 8.03 | 10.63 | 7.42 | −1.31 |
| Snx7 | 7.54 | 1.56 | 2.61 | 2.29 | 1.93 | 6.46 | 8.82 | 1.94 | 1.96 | 4.07 | 2.49 | −1.31 |
| Kank4 | 2.87 | 0.53 | 0.69 | 0.44 | 1.47 | 6 | 2.38 | 0.22 | 0.34 | 0.13 | 0.27 | −1.31 |
| Csrnp3 | 2.05 | 0.04 | 0.29 | 0.18 | 0.08 | 0.11 | 0.26 | 0.01 | 0.02 | 0.01 | 0 | −1.30 |
| Gnb4 | 14.04 | 4.04 | 5.59 | 4.59 | 4.67 | 9.39 | 9.62 | 7.5 | 7.6 | 3.31 | 3.31 | −1.30 |
| Anxa7 | 17.47 | 5.42 | 6.86 | 6.1 | 6.99 | 22.72 | 20.91 | 22.65 | 22.78 | 25.13 | 17.44 | −1.30 |
| Parm1 | 2.75 | 0.16 | 0.75 | 0.29 | 0.7 | 2.61 | 0.62 | 0.85 | 4.69 | 10.6 | 5.65 | −1.30 |
| Mfap5 | 2.39 | 0.25 | 0.27 | 0.48 | 0.45 | 12.01 | 6.73 | 0 | 0 | 0.07 | 0.11 | −1.30 |
| Rhd | 1.65 | 0.06 | 0.07 | 0.08 | 0.18 | 0.06 | 0.14 | 0.34 | 0.12 | 0.13 | 0.1 | −1.30 |
| Col9a1 | 16.92 | 3.45 | 5.69 | 6.85 | 4.16 | 3.93 | 9.17 | 0.51 | 1.01 | 0.17 | 0.09 | −1.30 |
| Pla2g4a | 12.15 | 2.9 | 4.18 | 4.49 | 2.65 | 11 | 19.14 | 6.93 | 7 | 7.74 | 6.85 | −1.30 |
| Ctnnd2 | 3.88 | 0.79 | 1.15 | 0.81 | 1.28 | 0.52 | 0.83 | 0.05 | 0.08 | 0.38 | 0.21 | −1.30 |
| Fbln1 | 68.5 | 36.92 | 31.7 | 22.71 | 39.39 | 50.88 | 26.77 | 46.57 | 75.05 | 66.53 | 72.5 | −1.30 |
| Gypa | 1.77 | 0 | 0.06 | 0.19 | 0 | 0.32 | 0.3 | 0 | 0 | 0 | 0 | −1.30 |
| Fzd6 | 3.97 | 0.71 | 1.35 | 0.69 | 0.88 | 2.24 | 3.76 | 0.01 | 0.28 | 1.18 | 1.51 | −1.30 |
| Sema5a | 5.32 | 1.07 | 1.65 | 1.49 | 1.01 | 3.98 | 6.26 | 11.97 | 7.95 | 4.88 | 3.69 | −1.30 |
| Notch3 | 16.12 | 8.5 | 6.59 | 5.34 | 9.19 | 8.62 | 3.41 | 9.76 | 10.01 | 6.78 | 9.5 | −1.30 |
| Fat4 | 10.51 | 3.95 | 4 | 3.38 | 3.75 | 3.43 | 2.99 | 3.04 | 3.93 | 2.01 | 1.46 | −1.30 |
| Ube2ql1 | 2.63 | 0.65 | 0.47 | 0.49 | 0.53 | 0.87 | 0.3 | 2.61 | 2.38 | 4.58 | 2.91 | −1.29 |
| Col11a1 | 6.9 | 1.5 | 2.16 | 2.31 | 1.6 | 1.58 | 1.41 | 0.03 | 0.02 | 0.05 | 0 | −1.29 |
| Tubb2a | 20.94 | 8.31 | 9.8 | 6.18 | 8.25 | 25.74 | 25.5 | 6.82 | 5.83 | 8.74 | 10 | −1.29 |
| Myo1d | 3.71 | 0.97 | 1.15 | 0.71 | 1.13 | 3.51 | 3.47 | 1.81 | 3.5 | 5.06 | 4.79 | −1.29 |
| Lcn2 | 1.44 | 0 | 0 | 0 | 0 | 4.01 | 10.23 | 0.62 | 1.7 | 5.14 | 5.03 | −1.29 |
| Cdh8 | 2.06 | 0.04 | 0.37 | 0.14 | 0.05 | 0.03 | 0 | 0 | 0 | 0 | 0 | −1.29 |
| Sgce | 24.66 | 8.63 | 10.04 | 9.04 | 6.14 | 19.09 | 14.92 | 33.69 | 32.67 | 30.64 | 31.55 | −1.28 |
| Dsg2 | 2.29 | 0.33 | 0.42 | 0.29 | 0.34 | 1.41 | 2.28 | 0.15 | 0.51 | 0.47 | 0.76 | −1.28 |
| Wnt7b | 1.62 | 0.02 | 0.13 | 0.03 | 0.61 | 10.74 | 4.2 | 0.35 | 0.7 | 1.03 | 0.83 | −1.28 |
| Wfdc2 | 196.85 | 95.28 | 87.08 | 74.07 | 98.11 | 310.39 | 422.49 | 17.04 | 90.05 | 777.18 | 683.21 | −1.28 |
| Veph1 | 1.54 | 0 | 0.09 | 0.01 | 0.07 | 1.31 | 1.87 | 0 | 0 | 0 | 0 | −1.27 |
| Ccdc3 | 2.02 | 0 | 0.44 | 0.06 | 0.51 | 8.57 | 5.87 | 0 | 0.06 | 0.03 | 0.06 | −1.27 |
| Loxl3 | 12.28 | 5.94 | 5.27 | 3.74 | 6.45 | 10.82 | 9.76 | 7.81 | 7.71 | 8.7 | 7.63 | −1.27 |
| Plod1 | 24.66 | 12.01 | 11.61 | 7.67 | 12.54 | 28.72 | 22.4 | 37.79 | 54.53 | 63.8 | 47 | −1.27 |
| Ear7 | 1.41 | 0 | 0 | 0 | 0 | 0 | 0 | 0 | 0 | 0 | 0 | −1.27 |
| Isl1 | 2.66 | 0 | 0.4 | 0.64 | 0 | 0.03 | 0 | 0.21 | 2.03 | 0.07 | 1.29 | −1.27 |
| Mcu | 30.01 | 10.64 | 12.53 | 11.23 | 9.05 | 12.23 | 8.59 | 11.33 | 10.29 | 6.66 | 8.68 | −1.27 |
| Kcnt2 | 2.02 | 0.16 | 0.29 | 0.22 | 0.17 | 0.73 | 0.95 | 0 | 0 | 0 | 0 | −1.27 |
| Frmd6 | 4.81 | 1.07 | 1.43 | 1.4 | 1.32 | 2.32 | 2.39 | 2.32 | 2.14 | 2.28 | 2.75 | −1.27 |
| Ppp1r36 | 4.17 | 0.86 | 1.24 | 1.06 | 1.12 | 2.02 | 2.05 | 0.75 | 1.07 | 1.14 | 0.87 | −1.27 |
| Sirt5 | 10.2 | 4.24 | 4.33 | 2.99 | 3.96 | 5.58 | 6.91 | 1.73 | 1.77 | 1.94 | 1.41 | −1.27 |
| Lix1l | 25.11 | 10.39 | 10.9 | 8.85 | 13.65 | 23.76 | 20.43 | 32.99 | 23.01 | 18.89 | 12.88 | −1.26 |
| Dpysl5 | 5.71 | 2.25 | 2.01 | 1.58 | 1.92 | 1.68 | 0.49 | 0.18 | 0.38 | 0.45 | 0.65 | −1.26 |
| Fam114a1 | 11.74 | 3.08 | 4.43 | 4.19 | 5.04 | 13.75 | 10.48 | 24.92 | 24.57 | 17.08 | 12.15 | −1.26 |
| Lsamp | 3.41 | 0.51 | 1.03 | 0.65 | 0.62 | 1.21 | 1.02 | 0.04 | 0.83 | 1.95 | 0.9 | −1.26 |
| Garnl3 | 7.71 | 2.92 | 2.96 | 2.31 | 3.73 | 5.07 | 3.41 | 2.48 | 2.72 | 3.41 | 3.25 | −1.26 |
| Chga | 2.06 | 0.27 | 0.43 | 0.13 | 0.28 | 0.27 | 0 | 0 | 0 | 0 | 0.08 | −1.26 |
| Slc27a6 | 4.93 | 1.54 | 1.56 | 1.41 | 1.23 | 1.74 | 2.41 | 0.78 | 0.69 | 0.49 | 0.57 | −1.25 |
| Lox | 3.46 | 0.09 | 0.94 | 0.8 | 0.49 | 5.63 | 20.49 | 29.223 | 8.05 | 3.77 | 2.39 | −1.25 |
| Gsn | 16.42 | 6.77 | 7.51 | 5.1 | 10.27 | 48.18 | 34.27 | 66.42 | 53.03 | 72.39 | 42.66 | −1.25 |
| Khdrbs2 | 1.99 | 0.2 | 0.15 | 0.36 | 0.07 | 0.32 | 0.52 | 0 | 0 | 0 | 0 | −1.25 |
| Tmem184a | 2.87 | 1.07 | 0.76 | 0.49 | 0.47 | 3.37 | 3.39 | 0.05 | 0.14 | 0.1 | 0.08 | −1.25 |
| Tmem30b | 1.82 | 0.02 | 0.2 | 0.17 | 0.03 | 2.08 | 8.01 | 0.23 | 0.28 | 0 | 0.09 | −1.25 |
| Arhgef5 | 7.78 | 2.58 | 3.17 | 2.23 | 3.24 | 8.6 | 6.59 | 1.12 | 4.11 | 8.21 | 5.23 | −1.25 |
| Dse | 10.6 | 1.26 | 3.81 | 3.98 | 1.24 | 5.08 | 9.29 | 13.79 | 10.24 | 6.25 | 5.21 | −1.24 |
| Susd1 | 7.27 | 2.56 | 2.99 | 1.99 | 2.21 | 4.4 | 3.92 | 1.61 | 2.6 | 1.24 | 2.25 | −1.24 |
| Akr1c14 | 1.63 | 0.03 | 0.2 | 0.02 | 0.14 | 0.17 | 0.15 | 0 | 0.04 | 0 | 0 | −1.24 |
| Flrt2 | 13.06 | 2.42 | 4.6 | 5.27 | 2.87 | 7.61 | 4.34 | 6.12 | 6.13 | 2.05 | 3 | −1.24 |
| Fzd8 | 7.52 | 2.34 | 2.9 | 2.3 | 2.3 | 2.81 | 3.64 | 1.76 | 2.73 | 1.7 | 1.21 | −1.24 |
| Astn2 | 1.84 | 0.24 | 0.31 | 0.09 | 0.28 | 0.75 | 0.39 | 0.01 | 0 | 0 | 0 | −1.24 |
| Dok5 | 2.57 | 0.2 | 0.68 | 0.34 | 0.15 | 0.47 | 0.19 | 1.74 | 3.01 | 3.45 | 1.73 | −1.24 |
| Itpripl2 | 5.19 | 1.29 | 1.76 | 1.48 | 1.5 | 3.53 | 6.46 | 7.48 | 5.91 | 5.12 | 4.38 | −1.24 |
| Kcne4 | 2 | 0.2 | 0.25 | 0.29 | 0.34 | 1.05 | 0.57 | 1.24 | 0.56 | 0.15 | 0.3 | −1.24 |
| Cnrip1 | 18.06 | 4.77 | 7.64 | 6.5 | 7.85 | 16.03 | 16.22 | 0.4 | 0.24 | 0 | 0 | −1.24 |
| Mycl | 10.97 | 3.92 | 4.1 | 4.04 | 3.82 | 6.39 | 3.05 | 0.54 | 0.6 | 0.51 | 1.28 | −1.24 |
| Egln3 | 3.78 | 0.47 | 1.06 | 0.99 | 0.38 | 11.96 | 10.47 | 87.79 | 29.6 | 66.86 | 52.82 | −1.24 |
| Vwa2 | 4.78 | 1.88 | 1.69 | 1.21 | 2.7 | 4.87 | 2.39 | 0.68 | 1.88 | 1.88 | 3.44 | −1.24 |
| Kcnj16 | 1.59 | 0 | 0.16 | 0.04 | 0.18 | 3 | 25.11 | 0.01 | 0 | 0 | 0.04 | −1.24 |
| Pdlim5 | 12.03 | 2.11 | 4.73 | 4.34 | 2.72 | 7.64 | 13.91 | 8.12 | 7.92 | 10.47 | 9.73 | −1.24 |
| Prickle1 | 12.71 | 3.4 | 4.95 | 4.7 | 4.18 | 8.5 | 7.83 | 5.34 | 5.96 | 6.13 | 5.23 | −1.23 |
| Fras1 | 5.86 | 2.19 | 1.92 | 1.91 | 2.26 | 3.64 | 3.19 | 0.95 | 1.36 | 1.54 | 1.54 | −1.23 |
| Arl4c | 25.52 | 8.79 | 11.92 | 8.63 | 10.03 | 14.8 | 16.52 | 4.33 | 4.83 | 13.2 | 11.37 | −1.23 |
| Prom2 | 1.48 | 0 | 0.1 | 0.01 | 0.02 | 3.29 | 2.01 | 0 | 0 | 0 | 0 | −1.23 |
| Slc6a9 | 8.7 | 4.08 | 3.8 | 2.47 | 6.58 | 7.22 | 6.33 | 11.84 | 18.11 | 14 | 13.23 | −1.23 |
| Atp1b2 | 3.48 | 0.95 | 0.91 | 0.91 | 1.18 | 4.26 | 5.79 | 16.29 | 12.97 | 10.84 | 6.87 | −1.23 |
| Arhgap28 | 18.28 | 6.25 | 6.89 | 7.55 | 5.49 | 7.27 | 4.74 | 9.63 | 11.82 | 6.48 | 9.38 | −1.23 |
| 4933412E12Rik | 2.56 | 0.65 | 0.66 | 0.38 | 0.71 | 1.54 | 3.06 | 0.31 | 0.96 | 2.17 | 1.15 | −1.23 |
| Tmem200a | 2.22 | 0.1 | 0.38 | 0.37 | 0.08 | 0.82 | 0.41 | 0.42 | 0.16 | 0 | 0.07 | −1.23 |
| Ednra | 14.42 | 3.53 | 5.3 | 5.87 | 4.43 | 7.09 | 14.27 | 11.54 | 9.2 | 5.17 | 5.9 | −1.23 |
| Snai2 | 43.33 | 6.12 | 15.48 | 20.45 | 5.11 | 14.06 | 23.14 | 82.05 | 43.76 | 20.31 | 18.76 | −1.22 |
| Chrd | 3.4 | 0.92 | 1.07 | 0.7 | 2.2 | 5.01 | 2.14 | 6.02 | 9.33 | 7.74 | 9.8 | −1.22 |
| Gstm2 | 22.88 | 11.92 | 8.95 | 9.56 | 13.63 | 43.27 | 43.91 | 53.97 | 61.41 | 131 | 109.49 | −1.22 |
| Pparg | 2.12 | 0.2 | 0.4 | 0.28 | 0.05 | 6.41 | 3.21 | 0.43 | 0.1 | 0 | 0.49 | −1.22 |
| Lgi3 | 2.03 | 0.36 | 0.41 | 0.2 | 0.31 | 0.95 | 0.58 | 0.03 | 0.11 | 0.06 | 0.05 | −1.22 |
| Tnk1 | 1.67 | 0.15 | 0.19 | 0.11 | 0.25 | 1.39 | 0.92 | 0.13 | 0.22 | 0.35 | 0.13 | −1.22 |
| Scnn1b | 1.6 | 0.03 | 0.22 | 0.02 | 0.69 | 33.79 | 23.07 | 0 | 0 | 0 | 0.05 | −1.22 |
| Sytl4 | 3.13 | 0.59 | 0.94 | 0.62 | 0.53 | 1.96 | 1.76 | 0 | 0 | 0 | 0 | −1.21 |
| Pkp3 | 2.27 | 0.52 | 0.57 | 0.25 | 0.29 | 2.54 | 1.55 | 0.34 | 0.45 | 0.41 | 0.1 | −1.21 |
| Ahnak | 1.99 | 0.09 | 0.3 | 0.28 | 0.29 | 3.02 | 3.66 | 5.59 | 4.34 | 5.16 | 2.46 | −1.21 |
| BC048679 | 1.41 | 0 | 0.08 | 0 | 0 | 0 | 0.1 | 0 | 0 | 0 | 0 | −1.21 |
| Arsj | 1.67 | 0.04 | 0.21 | 0.1 | 0.11 | 0.23 | 0.12 | 0 | 0 | 0 | 0 | −1.21 |
| Lmo4 | 69.21 | 23.52 | 27.17 | 31.61 | 25.02 | 43.97 | 46.15 | 30.71 | 33.23 | 36.02 | 34.78 | −1.21 |
| Cpne5 | 2.59 | 0.55 | 0.77 | 0.34 | 0.75 | 1.39 | 0.41 | 0.89 | 1.32 | 0.36 | 0.49 | −1.21 |
| Soat1 | 13.59 | 2.93 | 4.68 | 5.97 | 2.76 | 6.64 | 8.93 | 15.46 | 10.14 | 10.64 | 12.9 | −1.21 |
| Dusp1 | 260.44 | 61.15 | 125.21 | 99.47 | 65.15 | 107.53 | 204.79 | 24.21 | 15.57 | 62.3 | 64.24 | −1.21 |
| Anxa4 | 9.7 | 3.33 | 4.01 | 3.28 | 3.67 | 15.2 | 27.12 | 40.19 | 39.89 | 50.45 | 41.85 | −1.20 |
| Hif3a | 7.87 | 2.37 | 3.1 | 2.63 | 2.75 | 6.19 | 3.4 | 9.23 | 4.03 | 2.29 | 4.08 | −1.20 |
| Sipa1l3 | 5.85 | 1.89 | 2.18 | 1.8 | 2.53 | 3.5 | 2.68 | 2.73 | 2.68 | 3.14 | 3.23 | −1.20 |
| Clcf1 | 4.24 | 1.87 | 1.51 | 1.08 | 3.67 | 6.29 | 6.45 | 6.67 | 5.89 | 10.19 | 5.54 | −1.19 |
| Gpc4 | 4.25 | 1.74 | 1.28 | 1.32 | 3.08 | 11.46 | 24.34 | 11.29 | 11.91 | 8.65 | 9.05 | −1.19 |
| Dclk1 | 6.8 | 0.91 | 2.16 | 2.68 | 1.18 | 2.56 | 6.47 | 3.73 | 1.98 | 1.91 | 2.4 | −1.19 |
| Fblim1 | 65.93 | 34.63 | 31.78 | 24.97 | 29.78 | 35.4 | 33.67 | 47.89 | 54.43 | 45.72 | 46.25 | −1.19 |
| Ror1 | 1.89 | 0.16 | 0.33 | 0.21 | 0.07 | 0.56 | 0.27 | 0.17 | 0.38 | 0.02 | 0.24 | −1.19 |
| Mn1 | 5.62 | 1.08 | 2.02 | 1.8 | 1.1 | 1.12 | 0.37 | 0.04 | 0.11 | 0.06 | 0.09 | −1.19 |
| Chst3 | 3.04 | 0.91 | 0.89 | 0.68 | 0.89 | 0.92 | 0.66 | 1.26 | 0.92 | 0.17 | 0.28 | −1.18 |
| Fuca2 | 11.11 | 3.15 | 4.73 | 3.98 | 4.29 | 10.98 | 20.58 | 8.94 | 7.68 | 6.9 | 6.97 | −1.18 |
| Tmem229a | 1.6 | 0.11 | 0.18 | 0.12 | 0.11 | 0.61 | 2.07 | 0.05 | 0 | 0.02 | 0 | −1.18 |
| Edn3 | 7.78 | 3.07 | 3.18 | 2.59 | 2.69 | 3.67 | 2.37 | 1.16 | 1.67 | 0.27 | 0.15 | −1.18 |
| S100a11 | 252.1 | 137.26 | 123.09 | 98.93 | 181.07 | 519.53 | 378.58 | 268.13 | 328.66 | 644.95 | 406.79 | −1.18 |
| Olfm1 | 20.95 | 9.37 | 9.7 | 7.74 | 12.89 | 21.15 | 14.8 | 36.02 | 20.98 | 19.9 | 18.4 | −1.18 |
| Trpm5 | 1.65 | 0.22 | 0.26 | 0.09 | 0.82 | 3.5 | 0.93 | 0.01 | 0 | 0.02 | 0 | −1.17 |
| Fhod3 | 3.25 | 0.88 | 1.07 | 0.7 | 1.66 | 2.9 | 6.97 | 0.81 | 1.2 | 1.3 | 1.78 | −1.17 |
| Sh3d21 | 12.03 | 4.85 | 5.21 | 4.35 | 5 | 9.46 | 6.96 | 4.73 | 3.73 | 4.68 | 2.41 | −1.17 |
| Muc1 | 2.28 | 0.26 | 0.5 | 0.41 | 0.95 | 5.98 | 16.45 | 0.44 | 0.39 | 1.6 | 1.27 | −1.17 |
| Bnc2 | 3.7 | 0.77 | 1.16 | 1.01 | 0.96 | 1.05 | 1.25 | 1.1 | 0.44 | 0.21 | 0.2 | −1.17 |
| Ube2l6 | 9.15 | 4.22 | 3.33 | 3.68 | 3.81 | 8.42 | 6.95 | 5.98 | 4.26 | 5.36 | 5.98 | −1.17 |
| Sox9 | 26.98 | 6.56 | 10.65 | 12.2 | 3.49 | 1.7 | 2.29 | 7.57 | 9.12 | 6.86 | 8.74 | −1.17 |
| Macc1 | 1.33 | 0.04 | 0.06 | 0.01 | 0 | 0.31 | 0.31 | 0 | 0 | 0 | 0 | −1.17 |
| Galnt3 | 1.63 | 0 | 0.21 | 0.13 | 0.07 | 2.9 | 3.71 | 0.04 | 0 | 0.02 | 0 | −1.17 |
| Fam159a | 2.6 | 2.21 | 0.73 | 0.48 | 1.79 | 3.31 | 2.33 | 0.77 | 2.04 | 0.84 | 0.2 | −1.17 |
| Serping1 | 35.69 | 22.65 | 16.7 | 14.11 | 20.9 | 56.47 | 64.03 | 12.21 | 20.35 | 15.69 | 17.52 | −1.16 |
| Gjb3 | 1.47 | 0.22 | 0.15 | 0.06 | 0.17 | 1.67 | 0.08 | 0 | 0.06 | 0.18 | 0.49 | −1.16 |
| Sdpr | 2.07 | 0.03 | 0.43 | 0.32 | 0.08 | 4.95 | 6.23 | 0.02 | 0.03 | 0.03 | 0.05 | −1.16 |
| Cldn1 | 1.82 | 0 | 0.21 | 0.32 | 0.2 | 5.46 | 11.97 | 0.69 | 1.45 | 0.33 | 0.31 | −1.16 |
| Slc2a13 | 1.95 | 0.17 | 0.4 | 0.25 | 0.17 | 0.66 | 1.73 | 0.02 | 0.04 | 0.05 | 0.04 | −1.15 |
| Gfra1 | 48.95 | 28.81 | 20.02 | 22.94 | 36.27 | 32.05 | 27.79 | 2.29 | 2.39 | 5.5 | 5.74 | −1.15 |
| Plet1 | 12.05 | 4.35 | 5.47 | 4.29 | 2.88 | 36.85 | 22.85 | 1.19 | 1.73 | 5.94 | 9.62 | −1.15 |
| Pde8b | 2.34 | 0.52 | 0.59 | 0.42 | 0.34 | 2.01 | 2.45 | 0.2 | 0.27 | 0.16 | 0.23 | −1.15 |
| Nfix | 1.55 | 0.08 | 0.24 | 0.06 | 0.48 | 3.51 | 4.67 | 0.09 | 0.13 | 0.88 | 0.57 | −1.15 |
| Hkdc1 | 1.37 | 0.14 | 0.13 | 0.01 | 0.24 | 0.75 | 0.44 | 0.01 | 0 | 0 | 0 | −1.15 |
| Pitpnc1 | 7.58 | 1.6 | 3.21 | 2.55 | 1.84 | 4.17 | 5.51 | 3.74 | 3.73 | 3.6 | 3.86 | −1.14 |
| Pappa | 1.53 | 0.13 | 0.18 | 0.11 | 0.12 | 0.46 | 0.59 | 0 | 0 | 0.01 | 0 | −1.14 |
| Kirrel | 16.39 | 6.09 | 7.25 | 6.5 | 6.7 | 9.01 | 8.91 | 8.22 | 8.89 | 7.58 | 7.9 | −1.14 |
| Clcnka | 1.44 | 0 | 0.16 | 0.05 | 0.14 | 3.44 | 46.37 | 0.08 | 0 | 0.11 | 0 | −1.14 |
| Ap3b2 | 1.35 | 0.02 | 0.1 | 0.03 | 0.09 | 0.02 | 0 | 0 | 0.11 | 0 | 0.08 | −1.14 |
| Wwtr1 | 12.87 | 3.25 | 5.27 | 5.32 | 3.36 | 9.14 | 22.77 | 22.89 | 15.57 | 15.09 | 14.06 | −1.14 |
| Fjx1 | 15.26 | 7.32 | 6.84 | 5.95 | 6.36 | 6.15 | 4.4 | 13.12 | 13.45 | 14.19 | 11.21 | −1.14 |
| Serpine2 | 32.32 | 14.34 | 14.82 | 13.49 | 14.01 | 38.22 | 68.44 | 48.01 | 34.94 | 45.57 | 53.33 | −1.14 |
| Hdac9 | 1.88 | 0.07 | 0.35 | 0.27 | 0.04 | 0.58 | 1.35 | 0.01 | 0 | 0 | 0 | −1.14 |
| Sfn | 2 | 0.22 | 0.42 | 0.31 | 0.5 | 24 | 4.15 | 0.76 | 2.29 | 3.32 | 1.93 | −1.14 |
| Ndufa4l2 | 2.24 | 0.4 | 0.55 | 0.4 | 0.41 | 8.23 | 16.03 | 82.1 | 25.12 | 45.78 | 14.73 | −1.14 |
| Psph | 23.21 | 11.11 | 10.22 | 9.83 | 14.1 | 15.16 | 13.62 | 18.64 | 20.42 | 19.15 | 17.06 | −1.13 |
| Negr1 | 5.34 | 0.56 | 1.75 | 2.03 | 1.23 | 2.49 | 6.85 | 9.63 | 3.9 | 3.9 | 2.62 | −1.13 |
| 5730409E04Rik | 5.15 | 1.71 | 2.02 | 1.59 | 2.41 | 3.65 | 3.67 | 1.67 | 1.55 | 1.64 | 0.92 | −1.13 |
| Maml2 | 5.73 | 0.98 | 2.26 | 1.88 | 1.13 | 2.17 | 4.33 | 1.65 | 1.56 | 0.86 | 0.8 | −1.13 |
| Phactr1 | 7.4 | 4.08 | 2.91 | 2.76 | 3.59 | 7.55 | 8.86 | 23.26 | 24.14 | 31.6 | 22.76 | −1.13 |
| Gpr126 | 1.33 | 0.01 | 0.11 | 0.02 | 0.1 | 1.83 | 0.95 | 1.16 | 0.44 | 0.01 | 0.13 | −1.13 |
| 2510009E07Rik | 4.01 | 0.53 | 1.43 | 1.15 | 0.88 | 3.25 | 5.06 | 1.22 | 0.98 | 1.65 | 1.56 | −1.13 |
| Fam101a | 2.39 | 1.11 | 0.68 | 0.42 | 1.4 | 2.93 | 1.64 | 0.25 | 0 | 0.35 | 0.38 | −1.13 |
| Bcat1 | 3.97 | 1.4 | 1.33 | 1.22 | 1.36 | 0.55 | 0.6 | 15.91 | 12.59 | 1.67 | 3.77 | −1.13 |
| Adrbk2 | 4.33 | 1.06 | 1.56 | 1.32 | 1.08 | 2.14 | 3.67 | 0.02 | 0.4 | 0.21 | 0.24 | −1.13 |
| Paqr6 | 1.29 | 0.05 | 0.1 | 0 | 0.21 | 4.61 | 0.72 | 0.61 | 0.32 | 0.06 | 0.37 | −1.12 |
| Snca | 2.5 | 0.93 | 0.51 | 0.7 | 0.95 | 1.96 | 0.84 | 0 | 0 | 0 | 0 | −1.12 |
| A330048O09Rik | 1.66 | 0 | 0.32 | 0.12 | 0.5 | 0.8 | 0.65 | 0 | 0 | 0 | 0 | −1.12 |
| Slc8a1 | 3.16 | 0.46 | 0.89 | 0.93 | 0.72 | 2.11 | 7.12 | 0.77 | 0.67 | 0.73 | 0.82 | −1.12 |
| Efna5 | 21.09 | 7.04 | 8.61 | 9.73 | 7.81 | 8.35 | 9.37 | 6.5 | 3.46 | 6.61 | 5.12 | −1.12 |
| Ddx58 | 2.42 | 0.32 | 0.62 | 0.53 | 0.54 | 2.56 | 3.84 | 0.25 | 0.27 | 0.69 | 0.4 | −1.12 |
| Peg10 | 38.67 | 8.62 | 17.4 | 17.14 | 3.45 | 4.21 | 3.4 | 89.28 | 81.15 | 25.15 | 49.6 | −1.12 |
| B130024G19Rik | 14.72 | 4.63 | 6.6 | 5.88 | 5.41 | 7.62 | 15.84 | 4.79 | 9.59 | 5.76 | 5.08 | −1.12 |
| Rras2 | 33.27 | 8.45 | 13.61 | 15.97 | 9.79 | 17.65 | 23.23 | 18.02 | 16.19 | 20.1 | 21.63 | −1.12 |
| Pxylp1 | 5.42 | 1.93 | 2.15 | 1.77 | 2.4 | 6.43 | 4.49 | 1.44 | 1.66 | 3.88 | 2.92 | −1.12 |
| Pls3 | 24.82 | 6.39 | 11.19 | 10.65 | 9.76 | 19.64 | 45.56 | 14.17 | 10.4 | 11.8 | 8.56 | −1.12 |
| Il1rap | 2.94 | 0.14 | 0.75 | 0.89 | 0.23 | 0.65 | 2.16 | 2.63 | 1.2 | 0.83 | 0.82 | −1.11 |
| Golt1a | 1.29 | 0 | 0.09 | 0.03 | 0.19 | 1.72 | 4.01 | 0 | 0 | 0 | 0 | −1.11 |
| Ear6 | 1.16 | 0 | 0 | 0 | 0 | 0 | 0 | 0 | 0 | 0 | 0 | −1.11 |
| Tmprss2 | 1.61 | 0.1 | 0.22 | 0.2 | 0.55 | 21.71 | 43.07 | 0.03 | 0 | 0.11 | 0.09 | −1.11 |
| Snhg18 | 21.25 | 9.48 | 9.19 | 9.55 | 8.26 | 28.18 | 33.48 | 38.08 | 38.69 | 34.18 | 24.59 | −1.10 |
| Pknox2 | 9.86 | 3.1 | 4.24 | 3.9 | 3.02 | 3.94 | 5.48 | 5.43 | 7.6 | 4.99 | 6.81 | −1.10 |
| Itk | 1.27 | 0.09 | 0.09 | 0.03 | 0.02 | 0.11 | 0 | 0 | 0 | 0 | 0 | −1.10 |
| Igf2r | 19.1 | 9.73 | 8.63 | 8.15 | 11.16 | 13.77 | 6.96 | 30.37 | 29.14 | 19.81 | 23.74 | −1.10 |
| 2510049J12Rik | 1.63 | 0.3 | 0.31 | 0.15 | 0.53 | 0.73 | 0.71 | 0.17 | 0.3 | 0.29 | 0.35 | −1.10 |
| Pak7 | 1.63 | 0.05 | 0.26 | 0.2 | 0.14 | 0.11 | 0.1 | 0.01 | 0 | 0 | 0 | −1.10 |
| Arhgap20 | 2.57 | 0.29 | 0.65 | 0.69 | 0.37 | 0.29 | 0.28 | 0.01 | 0.06 | 0.03 | 0.11 | −1.10 |
| Cast | 6.96 | 1.54 | 2.74 | 2.71 | 2.6 | 6.49 | 8.11 | 4.44 | 4.45 | 4.96 | 3.9 | −1.10 |
| Thbs2 | 1.67 | 0.08 | 0.26 | 0.24 | 0.3 | 5.5 | 5.8 | 0.01 | 0.04 | 0.1 | 0.14 | −1.09 |
| Lonrf1 | 10.98 | 3.05 | 4.26 | 4.96 | 2.48 | 2.09 | 4 | 21.54 | 9.31 | 6.83 | 9.5 | −1.09 |
| Bhlhe40 | 3.89 | 0.79 | 1.29 | 1.29 | 1.28 | 6.9 | 28.32 | 109.92 | 37.38 | 52.8 | 49.01 | −1.09 |
| Gjb1 | 1.23 | 0 | 0.09 | 0 | 0.24 | 7.59 | 3.97 | 0 | 0 | 0 | 0 | −1.09 |
| Adamts6 | 2.03 | 0 | 0.38 | 0.46 | 0.3 | 0.34 | 0.33 | 0 | 0 | 0 | 0 | −1.09 |
| Lrfn5 | 1.42 | 0.04 | 0.14 | 0.13 | 0.02 | 0.49 | 0.02 | 0 | 0 | 0 | 0 | −1.09 |
| Ell3 | 7.4 | 2.61 | 2.92 | 2.96 | 2.54 | 3.34 | 3.41 | 0.91 | 1.61 | 2.21 | 2.96 | −1.09 |
| Msrb2 | 26.76 | 15.02 | 12.64 | 11.41 | 13.78 | 23.59 | 21.24 | 13.27 | 15.16 | 18.36 | 15.74 | −1.09 |
| Mfap3l | 1.94 | 0.15 | 0.41 | 0.35 | 0.24 | 0.7 | 3.16 | 0.47 | 0.46 | 0.32 | 0.43 | −1.09 |
| Ntng1 | 1.47 | 0.09 | 0.14 | 0.18 | 0.11 | 0.54 | 1.34 | 0 | 0 | 0 | 0.07 | −1.09 |
| Serpinb9 | 1.85 | 0.07 | 0.42 | 0.26 | 0.15 | 2.12 | 4.79 | 0.78 | 0.54 | 0.86 | 0.81 | −1.09 |
| Mbnl3 | 5.27 | 0.73 | 1.79 | 2.11 | 1.13 | 1.26 | 2.33 | 3.26 | 1.29 | 0.55 | 0.43 | −1.09 |
| Rab3ip | 22.42 | 10.77 | 10.15 | 9.92 | 12.18 | 19.55 | 29.51 | 4.07 | 4.81 | 11.65 | 13.31 | −1.09 |
| Sdk1 | 1.65 | 0.45 | 0.31 | 0.19 | 0.64 | 1.62 | 0.66 | 0 | 0.06 | 0.2 | 0.21 | −1.08 |
| Ermap | 1.16 | 0 | 0.02 | 0.02 | 0.02 | 0.09 | 0.2 | 0.04 | 0.07 | 0.06 | 0.03 | −1.08 |
| Lbh | 123.64 | 72.94 | 60.22 | 55.5 | 52.36 | 55.81 | 47.37 | 14.97 | 20.41 | 13.84 | 13.26 | −1.08 |
| Zcchc12 | 6.46 | 1.74 | 2.63 | 2.44 | 2.03 | 1.95 | 2.79 | 1.68 | 1.94 | 1.22 | 0.62 | −1.08 |
| B3gnt9 | 3.09 | 0.62 | 0.89 | 0.99 | 1.37 | 4.49 | 1.91 | 4.51 | 4.11 | 2.37 | 1.48 | −1.08 |
| Fhl3 | 13.9 | 5.34 | 6.02 | 6.14 | 5.3 | 8.03 | 4.15 | 23.66 | 30.76 | 28.5 | 22.25 | −1.07 |
| Snora24 | 1.1 | 0 | 0 | 0 | 0 | 0 | 0 | 0 | 0 | 0 | 0 | −1.07 |
| Tmem102 | 1.1 | 0 | 0 | 0 | 0.17 | 1.45 | 1.63 | 0.13 | 0.01 | 1.27 | 0 | −1.07 |
| Mr1 | 2.33 | 0.3 | 0.66 | 0.52 | 0.4 | 3.64 | 5.44 | 1.85 | 2.15 | 3.11 | 2.36 | −1.07 |
| Col24a1 | 2.17 | 0.19 | 0.52 | 0.51 | 0.11 | 0.38 | 0.83 | 0.15 | 0.09 | 0.14 | 0.16 | −1.07 |
| Ctps2 | 12.13 | 4.73 | 5.31 | 5.26 | 6.16 | 9.65 | 9.65 | 3.05 | 3.19 | 4.94 | 2.94 | −1.06 |
| Ccdc80 | 32.45 | 17.09 | 15.32 | 14.73 | 12.43 | 23.99 | 22.62 | 6.75 | 9.35 | 3.27 | 7.27 | −1.06 |
| Fxyd3 | 1.25 | 1.17 | 0.09 | 0.07 | 0.47 | 63.89 | 6.05 | 0 | 0.37 | 0 | 0.59 | −1.06 |
| 4930562F07Rik | 1.08 | 0.07 | 0 | 0 | 0.02 | 0.01 | 0.98 | 0 | 0 | 0 | 0 | −1.06 |
| Tmem171 | 1.67 | 0.24 | 0.28 | 0.29 | 0.57 | 6.64 | 9.84 | 0.05 | 0.17 | 1.32 | 1.72 | −1.06 |
| Dip2a | 5.27 | 1.78 | 2.02 | 2.02 | 2.27 | 4.35 | 5.28 | 3.78 | 3.7 | 3.38 | 2.88 | −1.05 |
| C1s1 | 1.79 | 0.23 | 0.35 | 0.34 | 0.8 | 5.44 | 9.02 | 1.02 | 0.76 | 0.68 | 0.91 | −1.05 |
| Adamts5 | 1.53 | 0.07 | 0.22 | 0.22 | 0.18 | 0.72 | 1.44 | 0.14 | 0.19 | 0.12 | 0.19 | −1.05 |
| Slc2a3 | 4.43 | 1.31 | 1.57 | 1.68 | 1.34 | 3.64 | 5.05 | 266.28 | 193.14 | 81.9 | 137.43 | −1.05 |
| Lrrk1 | 3.01 | 0.83 | 1 | 0.89 | 1.83 | 3.76 | 1.64 | 2.52 | 3.02 | 3.28 | 3.21 | −1.04 |
| Casp4 | 1.63 | 0 | 0.29 | 0.27 | 0.06 | 3.27 | 5.9 | 5.33 | 1.54 | 1.9 | 0.89 | −1.04 |
| Al593442 | 1.29 | 0.01 | 0.1 | 0.13 | 0 | 0 | 0.02 | 0.02 | 0.03 | 0 | 0 | −1.04 |
| Tcp11 | 2.08 | 0.75 | 0.47 | 0.54 | 0.91 | 2.79 | 2.15 | 0.17 | 0.51 | 0.22 | 0.53 | −1.03 |
| Cntn1 | 1.27 | 0.06 | 0.12 | 0.13 | 0.04 | 0.12 | 0.23 | 0.04 | 0 | 0 | 0 | −1.01 |
| Fam162b | 1.01 | 0 | 0 | 0 | 0 | 0.63 | 0.43 | 0 | 0 | 0 | 0 | −1.01 |
| Mir7036 | 139.12 | 0 | 0 | 281.33 | 0 | 377.28 | 0 | 678.16 | 0 | 0 | 1382.52 | 0.02 |
| DQ267102 | 79.3 | 0 | 172.62 | 0 | 0 | 0 | 0 | 0 | 0 | 0 | 0 | 0.12 |
| Mir6945 | 206.64 | 867.99 | 0 | 522.31 | 292.8 | 1400.89 | 142.28 | 167.87 | 0 | 0 | 0 | 0.34 |
| Snora75 | 114.93 | 85.83 | 50.04 | 271.15 | 43.43 | 0 | 137.17 | 136.95 | 0 | 116.64 | 114.21 | 0.48 |
| Moap1 | 5.33 | 1.09 | 1.17 | 17.06 | 0.95 | 0 | 8.36 | 4.34 | 1.92 | 2.71 | 8.19 | 0.68 |
| Mir1839 | 67.46 | 188.91 | 220.27 | 0 | 0 | 182.93 | 464.48 | 0 | 0 | 0 | 0 | 0.70 |
| Zfp516 | 11.34 | 24.82 | 24.28 | 24.03 | 22.84 | 17.9 | 16.85 | 18.91 | 21.14 | 18.4 | 20.03 | 1.03 |
| Crhr1 | 0.05 | 2.21 | 1.18 | 1.11 | 0.94 | 0.57 | 0 | 0 | 0 | 0.3 | 0.43 | 1.03 |
| Cap2 | 3.75 | 12.15 | 8.53 | 8.92 | 13.18 | 10.59 | 4.93 | 3.58 | 2.62 | 4.13 | 3.75 | 1.03 |
| Ubxn10 | 0.56 | 2.33 | 2.17 | 2.26 | 1.88 | 0.95 | 1.05 | 0.67 | 1.1 | 1.24 | 1.66 | 1.04 |
| Dll1 | 0.71 | 2.38 | 2.56 | 2.52 | 2.94 | 2.54 | 2.36 | 0.18 | 0.2 | 0.34 | 1.18 | 1.05 |
| Phlda3 | 22.26 | 66.95 | 49.23 | 45.6 | 68.12 | 75.66 | 36.84 | 167.62 | 160.98 | 267.9 | 234.02 | 1.06 |
| Mir7052 | 139.12 | 0 | 302.85 | 281.33 | 0 | 0 | 0 | 226.05 | 0 | 0 | 0 | 1.06 |
| Arrdc4 | 6.55 | 17.96 | 15.1 | 15.04 | 14.16 | 20.9 | 27.31 | 19.71 | 37.65 | 30.35 | 25.54 | 1.09 |
| Npm3 | 55.92 | 150.98 | 117.54 | 123.46 | 137.4 | 107.02 | 80.17 | 70.43 | 69.79 | 76.16 | 89.85 | 1.09 |
| Gstk1 | 19.21 | 56.64 | 41.16 | 43.19 | 57.32 | 58.74 | 44.12 | 45.56 | 29.91 | 44.92 | 26.81 | 1.10 |
| Nell2 | 3.27 | 11.5 | 7.81 | 8.45 | 9.56 | 5.42 | 4.42 | 20.76 | 21.77 | 4.81 | 10.61 | 1.10 |
| Lrp8 | 1.67 | 4.52 | 4.37 | 5.15 | 4.08 | 2.55 | 1.38 | 12.47 | 14.9 | 14.91 | 14.3 | 1.11 |
| Sipa1l2 | 4.66 | 11.95 | 11 | 11.46 | 9.9 | 7.76 | 7.67 | 9.76 | 11.92 | 16.54 | 15.47 | 1.11 |
| Wt1 | 61.74 | 93.51 | 127.33 | 142.12 | 90.09 | 72.64 | 160.65 | 122.06 | 76.13 | 99.32 | 80.43 | 1.11 |
| Dio3os | 0.1 | 1.93 | 1.36 | 1.42 | 1.08 | 0.69 | 0.53 | 0.58 | 2.34 | 1.63 | 0.51 | 1.12 |
| Barx2 | 0.09 | 1.98 | 1.18 | 1.57 | 1.03 | 0.28 | 0.48 | 0.67 | 0.3 | 0.32 | 0.09 | 1.12 |
| Ctdspl | 11.09 | 36.11 | 26.45 | 24.43 | 39.56 | 38.02 | 31.86 | 14.37 | 16.51 | 29.24 | 26.54 | 1.13 |
| Ramp2 | 67.96 | 237.04 | 162.26 | 137.54 | 287.42 | 381.02 | 206.81 | 95.02 | 157.1 | 241.1 | 206.42 | 1.13 |
| Zfp503 | 35.69 | 55.34 | 75.79 | 83.05 | 50.16 | 39.39 | 44.34 | 36.39 | 29.42 | 33.82 | 32.71 | 1.13 |
| Cecr2 | 2.51 | 5.13 | 6.33 | 7.16 | 3.68 | 2.24 | 4.7 | 1.03 | 1.45 | 0.89 | 2 | 1.14 |
| Hoxd12 | 9.49 | 28.23 | 20.23 | 24.15 | 20.17 | 11.67 | 5.62 | 32.28 | 16.35 | 3.19 | 2.57 | 1.14 |
| Pcp2 | 0.28 | 1.81 | 1.96 | 1.71 | 2.16 | 1.15 | 1.14 | 0.15 | 0.3 | 0 | 0.52 | 1.15 |
| Arid3b | 6.81 | 20.82 | 15.48 | 17.15 | 12.77 | 3.65 | 2.81 | 6.81 | 8.26 | 1.82 | 8.93 | 1.15 |
| Ocm | 0 | 3.61 | 1.29 | 1.15 | 5.56 | 11.89 | 6.04 | 0.08 | 0 | 0 | 0 | 1.15 |
| Gm11240 | 0.11 | 0.95 | 1.57 | 1.37 | 0.96 | 1.22 | 0.98 | 0.37 | 0.49 | 0 | 0 | 1.15 |
| Nfe2 | 2.86 | 10.01 | 8.15 | 7.04 | 7.21 | 3.6 | 1.73 | 1.46 | 3.06 | 4.33 | 2.32 | 1.15 |
| Hapln3 | 3.05 | 15.11 | 8.71 | 7.35 | 17.82 | 10.28 | 6.45 | 1.23 | 4.03 | 4.62 | 5.76 | 1.16 |
| Olfr1372-ps1 | 0.58 | 1.96 | 2.8 | 2.27 | 1.54 | 4.09 | 14.31 | 14.66 | 23.6 | 61.13 | 43.87 | 1.16 |
| 1110046J04Rik | 2.39 | 5.89 | 6.61 | 6.62 | 6.64 | 12.45 | 7.31 | 3.96 | 3.29 | 7.71 | 7.36 | 1.17 |
| Adhfe1 | 0.58 | 1.88 | 2.33 | 2.8 | 2.17 | 2.7 | 10.41 | 0.95 | 1.63 | 1.56 | 1.76 | 1.17 |
| Cpa5 | 0.16 | 2.44 | 1.53 | 1.73 | 2.34 | 1.31 | 0.77 | 1.57 | 1.92 | 0.33 | 0.85 | 1.18 |
| Nrsn1 | 0.74 | 2.74 | 2.65 | 3.27 | 2.17 | 0.69 | 0 | 0.07 | 0.08 | 0 | 0.14 | 1.19 |
| Ube2cbp | 5.23 | 14.74 | 12.16 | 14.21 | 13.2 | 10.39 | 7.74 | 5.21 | 6.4 | 7.04 | 7.13 | 1.19 |
| Stk32a | 0.1 | 1.55 | 1.42 | 1.59 | 1.68 | 0.86 | 0.82 | 1.83 | 1.44 | 0.59 | 0.88 | 1.19 |
| Fam107a | 1.45 | 3.47 | 4.98 | 4.18 | 3.04 | 10.36 | 16.67 | 1.41 | 1.42 | 1.48 | 1.57 | 1.19 |
| Klhdc8a | 0.04 | 2.08 | 1.28 | 1.49 | 3.52 | 9.34 | 14.96 | 9.96 | 16.07 | 43.88 | 32.19 | 1.20 |
| Crb2 | 0.43 | 2.8 | 2.23 | 2.33 | 2.9 | 1.51 | 1.07 | 1.28 | 2.15 | 2.14 | 2.79 | 1.20 |
| Ust | 3.03 | 7.84 | 7.17 | 9.32 | 6.32 | 5.48 | 5.49 | 3.34 | 3.59 | 3.37 | 3.07 | 1.20 |
| Ppef2 | 0.08 | 1.67 | 1.38 | 1.58 | 1.31 | 0.86 | 0.82 | 2.52 | 3.65 | 1.81 | 3.88 | 1.20 |
| Cd74 | 0.62 | 5.67 | 3.21 | 2.24 | 2.29 | 8.95 | 23.84 | 0.35 | 0.93 | 17.9 | 9.68 | 1.20 |
| Thsd4 | 6.44 | 21.41 | 15.68 | 16.56 | 22.13 | 15.51 | 12.6 | 13.19 | 13.35 | 26.37 | 22.61 | 1.20 |
| Dtnb | 20.17 | 63.03 | 47.83 | 47.69 | 57.96 | 44.22 | 33.9 | 25.58 | 31.02 | 59.32 | 57.46 | 1.20 |
| Hoxa11 | 31.81 | 75.25 | 73.39 | 76.31 | 74.94 | 57.47 | 48.27 | 116.33 | 112.56 | 89.99 | 99.69 | 1.21 |
| Nuak1 | 4.29 | 10.98 | 11 | 11.65 | 11.41 | 8.92 | 11.39 | 5.14 | 6.44 | 25.22 | 24.5 | 1.22 |
| Sncaip | 18.37 | 50.39 | 44.47 | 43.97 | 51.27 | 46.25 | 44.65 | 14.71 | 23.61 | 25.37 | 20.87 | 1.22 |
| Aldh1b1 | 1.24 | 5.89 | 4.72 | 3.74 | 4.44 | 3.44 | 2.55 | 19.1 | 20.55 | 14.87 | 10.19 | 1.22 |
| Glt28d2 | 0.32 | 1.91 | 1.73 | 2.46 | 1.23 | 1.14 | 3.46 | 0.69 | 0.78 | 1.25 | 1.02 | 1.23 |
| Gatsl3 | 1.1 | 4.74 | 4.04 | 3.83 | 4.1 | 5.03 | 2.7 | 1.82 | 2.52 | 4.47 | 6.69 | 1.23 |
| Slit1 | 1.19 | 5.87 | 4.69 | 3.65 | 5.37 | 3.45 | 2 | 0.17 | 0.53 | 0.53 | 1.03 | 1.24 |
| Mafb | 6.38 | 14.89 | 15.68 | 17.2 | 8.02 | 4.78 | 11.08 | 3.41 | 5.41 | 7.86 | 10.69 | 1.24 |
| Rai2 | 1.77 | 3.96 | 5.63 | 5.51 | 3.64 | 3.2 | 3.14 | 1.58 | 2.54 | 3.17 | 2.12 | 1.25 |
| Cdh22 | 0.21 | 3.09 | 2.28 | 1.47 | 2.82 | 2.79 | 4.25 | 0.03 | 0.19 | 1.6 | 1.01 | 1.25 |
| Svopl | 0.86 | 4.05 | 3.74 | 3.1 | 3.99 | 2.41 | 5.99 | 0.16 | 0.29 | 0.15 | 1.01 | 1.25 |
| Gfra2 | 1.49 | 4.4 | 4.36 | 5.5 | 5.32 | 7.6 | 9.46 | 2.16 | 7.99 | 38.51 | 32.56 | 1.25 |
| Pgbd5 | 0.83 | 4.8 | 3.35 | 3.38 | 3.5 | 2.24 | 1.08 | 6.76 | 6.25 | 11.56 | 13.72 | 1.25 |
| Scara3 | 14.57 | 58.23 | 37.16 | 35.22 | 62.6 | 35.27 | 22.21 | 47.91 | 71.61 | 78.77 | 87.54 | 1.26 |
| Gas1 | 72.26 | 141.74 | 161.56 | 188.31 | 120.54 | 91.93 | 106.69 | 130.62 | 101.68 | 117.55 | 82.71 | 1.26 |
| Gm19705 | 0.13 | 0.74 | 1.29 | 2.14 | 0.75 | 1.79 | 8.37 | 5.8 | 7.21 | 3.22 | 4.6 | 1.26 |
| Camk2a | 1.26 | 6.32 | 4.51 | 4.36 | 4.93 | 2.43 | 2.9 | 2.01 | 1.87 | 0.39 | 0.84 | 1.27 |
| Acy3 | 6.94 | 30.36 | 19.31 | 16.93 | 28.69 | 31.69 | 21.84 | 11.17 | 15.57 | 17.6 | 18.97 | 1.27 |
| Tsc22d1 | 378.4 | 871.54 | 829.74 | 1001.01 | 898.48 | 855.17 | 840.98 | 185.25 | 250.24 | 570.55 | 549.87 | 1.27 |
| Ntsr1 | 8.62 | 34.03 | 21.87 | 22.71 | 33.85 | 21.64 | 11.17 | 7.28 | 14.42 | 38.77 | 31.8 | 1.28 |
| Fam160a1 | 0.64 | 3.39 | 2.6 | 3.36 | 3.1 | 2.56 | 4.12 | 1.69 | 2.2 | 2.43 | 2.41 | 1.28 |
| Sct | 8.3 | 33.44 | 20.8 | 22.41 | 26.51 | 30.02 | 14.3 | 142.65 | 313.11 | 433.23 | 519.22 | 1.28 |
| Slco3a1 | 4.85 | 19.2 | 13.35 | 13.11 | 13.1 | 9.77 | 9.34 | 0.97 | 2.02 | 12.09 | 14.36 | 1.28 |
| Casz1 | 0.62 | 3.15 | 2.99 | 2.91 | 2.95 | 1.97 | 1.56 | 0.12 | 0.4 | 0.78 | 0.86 | 1.29 |
| Col13a1 | 2.42 | 9.36 | 8.21 | 6.51 | 7.4 | 7.55 | 10.88 | 10.09 | 11 | 16.03 | 13.48 | 1.29 |
| 2210011C24Rik | 0.35 | 2.18 | 2.04 | 2.56 | 1.1 | 0.95 | 1.72 | 1.71 | 3.47 | 0.95 | 1.94 | 1.29 |
| Acsbg1 | 0.86 | 6.12 | 3.91 | 3.22 | 5.68 | 3.79 | 0.88 | 0.48 | 0.64 | 0.26 | 0.26 | 1.30 |
| Asic2 | 2.09 | 5.7 | 7.18 | 6.02 | 6.33 | 7.56 | 7.71 | 6.86 | 12.58 | 7.6 | 11.22 | 1.30 |
| Zhx2 | 5.1 | 16.58 | 15.19 | 12.84 | 16.89 | 12.37 | 8.48 | 4.42 | 6.51 | 5.94 | 6.82 | 1.30 |
| Sema3f | 24.22 | 94.48 | 67.89 | 54.72 | 107.64 | 76.13 | 47.05 | 49.19 | 68.3 | 194.62 | 178.13 | 1.30 |
| Cacna2d1 | 6.19 | 6.74 | 14.12 | 19.42 | 6.69 | 4.04 | 11.63 | 4.56 | 4.1 | 3.34 | 3.2 | 1.31 |
| Il17rd | 3.74 | 10.73 | 10.23 | 11.24 | 9.86 | 8.97 | 12.59 | 7.12 | 8.1 | 14.56 | 16.27 | 1.31 |
| Al464131 | 1.43 | 7.02 | 4.87 | 5.27 | 7.61 | 7.21 | 5.12 | 5.18 | 6.04 | 11.62 | 9.91 | 1.32 |
| Fbxo6 | 4.52 | 17.66 | 13.6 | 11.98 | 19.31 | 22.77 | 26.07 | 43.62 | 53.1 | 104.2 | 89.86 | 1.32 |
| Rem2 | 4.56 | 12.13 | 14.85 | 10.95 | 10.34 | 6.53 | 3.72 | 9.63 | 9.91 | 16.75 | 14.8 | 1.32 |
| Gbx2 | 0.11 | 1.4 | 1.69 | 1.86 | 0.53 | 0 | 0 | 1.97 | 5.45 | 1.39 | 4.32 | 1.32 |
| Cep152 | 4.06 | 12.47 | 10.93 | 12.47 | 10.62 | 7.67 | 5.77 | 14.4 | 8.54 | 9.13 | 10.3 | 1.33 |
| Fam189b | 14.08 | 57.58 | 37.44 | 36.55 | 58.29 | 48.31 | 31.09 | 36.87 | 39.8 | 60.61 | 57.28 | 1.33 |
| Snord72 | 253.52 | 0 | 0 | 1281.64 | 0 | 0 | 0 | 0 | 0 | 0 | 0 | 1.33 |
| Myc | 32.86 | 97.14 | 90.13 | 78.91 | 87.6 | 64.97 | 37.72 | 22.12 | 31.48 | 16.65 | 24.64 | 1.34 |
| Hes6 | 30.91 | 124.56 | 91.16 | 68.49 | 119.26 | 73.32 | 40.94 | 51.05 | 56.42 | 72.28 | 73.31 | 1.34 |
| Dusp2 | 6.38 | 13.19 | 18.1 | 17.44 | 13.62 | 15.71 | 18.17 | 12.47 | 5.73 | 41.18 | 34.57 | 1.35 |
| Pdgfa | 8.75 | 32.64 | 26.73 | 21.11 | 29.77 | 31.79 | 36.15 | 2.42 | 4.07 | 6.89 | 7.72 | 1.35 |
| Chst2 | 5.17 | 16.33 | 14.9 | 14.64 | 13.09 | 8.3 | 3.48 | 8.86 | 7.06 | 11.39 | 6.88 | 1.35 |
| Mir1894 | 38.57 | 0 | 83.95 | 116.98 | 109.29 | 0 | 159.32 | 62.66 | 0 | 117.41 | 0 | 1.36 |
| Cxcr4 | 19.98 | 43.85 | 48.59 | 57.79 | 44.04 | 33.42 | 49.78 | 21.29 | 20.53 | 101.04 | 79.15 | 1.37 |
| Bmp7 | 21.13 | 78.16 | 57.93 | 54.57 | 92.74 | 64.43 | 46.73 | 3.13 | 10.81 | 12.4 | 13.95 | 1.37 |
| Psd2 | 2.89 | 6.05 | 11.44 | 6.82 | 1.78 | 1.5 | 1.02 | 0.38 | 0.7 | 0.93 | 2.06 | 1.38 |
| Ffar4 | 0.81 | 4.19 | 3.49 | 3.94 | 2.55 | 0.69 | 2.57 | 6.55 | 7.41 | 21.68 | 23.24 | 1.38 |
| Gldc | 4.52 | 21.11 | 12.73 | 14.18 | 20.97 | 17.83 | 11.16 | 11.02 | 9.67 | 18.73 | 17.03 | 1.39 |
| Ctxn3 | 0.82 | 2.18 | 3.53 | 4.04 | 3.55 | 6.91 | 12.5 | 0.04 | 0.06 | 1.38 | 1.4 | 1.39 |
| Pgf | 14.85 | 72.91 | 42.38 | 39.7 | 66.63 | 37.53 | 2.8 | 39.44 | 62.52 | 36.7 | 25.46 | 1.41 |
| Adh1 | 12.05 | 43.8 | 31.99 | 35.6 | 13.88 | 63.65 | 31.02 | 2.8 | 11.35 | 7.38 | 5.06 | 1.41 |
| Ccl4 | 0.72 | 0.5 | 4.51 | 2.67 | 0.85 | 15.31 | 48.47 | 0 | 0 | 0 | 0 | 1.42 |
| Hey1 | 9.53 | 19.33 | 24 | 30.39 | 17.54 | 14.56 | 24.87 | 20.94 | 10.97 | 32.44 | 39.74 | 1.42 |
| Gdnf | 19.59 | 54.61 | 56.16 | 52.28 | 67.14 | 37.55 | 52.74 | 42.44 | 67.69 | 85.22 | 72.39 | 1.42 |
| Fam124a | 1.05 | 6.2 | 4.19 | 4.81 | 5.21 | 3.72 | 4.29 | 0.5 | 0.73 | 1.22 | 1.79 | 1.42 |
| Dst | 2.2 | 10.36 | 7.35 | 7.88 | 11.34 | 8.07 | 8.31 | 2.48 | 2.77 | 3.86 | 2.91 | 1.43 |
| Fgf9 | 1.46 | 2.4 | 4.22 | 7.03 | 2.49 | 2.76 | 7.32 | 2.23 | 2.28 | 21.92 | 18.44 | 1.43 |
| Has2os | 1.26 | 3.76 | 5.54 | 4.64 | 5.4 | 9 | 6.25 | 1.4 | 2.82 | 3.41 | 0.99 | 1.43 |
| Spag11b | 0.44 | 2.63 | 2.31 | 3.47 | 7.24 | 7.95 | 10.4 | 8.52 | 28.28 | 42.12 | 37.24 | 1.43 |
| 2010111I01Rik | 15.73 | 49.09 | 40.82 | 48.14 | 51.49 | 48.22 | 45.44 | 3.72 | 8.59 | 7.59 | 7.59 | 1.44 |
| Tcf24 | 0.23 | 1.69 | 1.87 | 2.82 | 1.4 | 1.15 | 4.72 | 19.45 | 18.22 | 12.13 | 9.8 | 1.44 |
| Slc9a3r1 | 14.04 | 69.99 | 40.87 | 38.95 | 78.24 | 66.44 | 36.51 | 15.14 | 19.83 | 48.8 | 41.21 | 1.44 |
| Ppp1r14a | 30.48 | 158.3 | 87.32 | 82.47 | 110.12 | 63.13 | 34.02 | 147.91 | 170.73 | 134.1 | 117.34 | 1.45 |
| Hoxa11os | 26.37 | 104.07 | 73.61 | 74.09 | 101.55 | 73.37 | 39.7 | 155.83 | 112.51 | 124.65 | 123.1 | 1.45 |
| Msc | 0.18 | 3.2 | 2.17 | 2.35 | 3.14 | 3.44 | 4.32 | 0.03 | 0.21 | 0.17 | 0.54 | 1.47 |
| Peli2 | 4.03 | 12.05 | 12.03 | 13.85 | 10.88 | 6.65 | 5.87 | 5.89 | 5.81 | 5.93 | 5.16 | 1.47 |
| Ankrd6 | 3.41 | 9.03 | 9.93 | 12.53 | 7.67 | 8.66 | 14.3 | 12.8 | 9.95 | 8.74 | 9.86 | 1.47 |
| Rcsd1 | 25.1 | 74.44 | 71.06 | 71.71 | 88.97 | 67.14 | 72.07 | 23.04 | 29.02 | 62.5 | 66.79 | 1.47 |
| St6galnac5 | 0.18 | 2.31 | 1.96 | 2.59 | 1.69 | 1.03 | 1.01 | 0.05 | 0.26 | 0 | 0.46 | 1.47 |
| 9030612E09Rik | 0.35 | 3.23 | 2.44 | 3.12 | 2.99 | 1.47 | 1.04 | 1.93 | 1.94 | 2.86 | 2.78 | 1.49 |
| Tnni2 | 0.71 | 7.95 | 4.31 | 3.29 | 10.23 | 13.64 | 9.12 | 1.06 | 1.53 | 0.9 | 1.47 | 1.49 |
| Rtn4rl2 | 0.28 | 3.62 | 2.68 | 2.51 | 5.65 | 3.86 | 1.28 | 29.93 | 27.41 | 34.11 | 41.39 | 1.49 |
| Mpeg1 | 0.27 | 1.54 | 3.42 | 1.74 | 1.17 | 10.43 | 32.74 | 0.48 | 1.96 | 4.77 | 2.41 | 1.50 |
| Snora43 | 22.35 | 73.01 | 77.02 | 52.72 | 137.21 | 30.3 | 51.29 | 145.23 | 149.72 | 45.35 | 92.52 | 1.50 |
| St3gal6 | 9.02 | 16.14 | 24.77 | 29.88 | 16.06 | 19.59 | 54.45 | 34.8 | 21.54 | 30.83 | 31.98 | 1.50 |
| Enc1 | 18.7 | 44.5 | 50.4 | 59.02 | 52.75 | 34.65 | 49.94 | 60.18 | 62.52 | 120.8 | 110.64 | 1.50 |
| Ptger4 | 1.55 | 4.94 | 4.93 | 7.53 | 3.69 | 0.97 | 2.85 | 2.68 | 2.21 | 1.42 | 1.29 | 1.50 |
| 5730408K05Rik | 6.53 | 38.28 | 24.3 | 16.43 | 31.85 | 18.53 | 15.69 | 27.14 | 37.94 | 12.95 | 18.11 | 1.50 |
| Proser2 | 1.26 | 5.07 | 4.89 | 6.02 | 3.42 | 3.7 | 5.53 | 5.22 | 3.29 | 6.38 | 6.61 | 1.51 |
| March3 | 0.69 | 5.05 | 4.21 | 3.47 | 4.76 | 4.55 | 2.99 | 0.29 | 0.81 | 0.91 | 2.11 | 1.52 |
| Fam132a | 28.21 | 97.54 | 93.69 | 72.05 | 89.05 | 114.48 | 100.63 | 99.32 | 178.91 | 306.79 | 270.7 | 1.52 |
| Bcl2l11 | 18.01 | 55.46 | 48.69 | 58.66 | 45.52 | 27.72 | 27.62 | 17.15 | 25.63 | 20.24 | 21.95 | 1.52 |
| Stc1 | 3.71 | 13.05 | 11.37 | 13.79 | 10.56 | 6.97 | 3.14 | 33.85 | 15.08 | 25.79 | 22.16 | 1.53 |
| Enox1 | 1.8 | 7.92 | 6.94 | 7.27 | 7.27 | 4.11 | 8.13 | 12.75 | 12.47 | 18.2 | 20.54 | 1.53 |
| Hoxb2 | 30.48 | 89.07 | 95.71 | 84.71 | 78.53 | 68.41 | 51.19 | 28.56 | 24.74 | 41.51 | 38.31 | 1.53 |
| Kcnmb2 | 0.33 | 1.84 | 2.41 | 3.3 | 1.43 | 1.92 | 3.6 | 0.07 | 0 | 0.28 | 0.1 | 1.54 |
| Eya4 | 3.9 | 7.43 | 11.57 | 14.93 | 6.96 | 4.9 | 10.17 | 3.12 | 3.17 | 2.96 | 4.06 | 1.54 |
| Spock1 | 0.18 | 2.82 | 2.22 | 2.68 | 2.07 | 0.5 | 0.55 | 0.03 | 0.51 | 0.5 | 1.25 | 1.55 |
| Anks1b | 0.47 | 2.29 | 2.96 | 3.65 | 1.74 | 0.13 | 0.17 | 1.14 | 0.16 | 0.06 | 0.09 | 1.55 |
| Cenpw | 17.51 | 44.94 | 40.29 | 66.17 | 39.3 | 28.69 | 22.07 | 41.87 | 36.45 | 20.98 | 24.98 | 1.55 |
| Tbx10 | 0.15 | 5.04 | 2.53 | 2.21 | 4.55 | 1.57 | 0.69 | 0.25 | 0.06 | 0.41 | 0.1 | 1.55 |
| Siglecg | 0.22 | 5.12 | 2.67 | 2.49 | 3.61 | 4.89 | 9.57 | 0.21 | 0.21 | 0.11 | 0.12 | 1.55 |
| Lst1 | 1.68 | 3.98 | 8.15 | 5.61 | 2.93 | 52.16 | 101.69 | 0.63 | 0.74 | 2.36 | 0 | 1.56 |
| Camk2n1 | 8.26 | 22.33 | 24.12 | 28.6 | 18.39 | 16.48 | 46 | 30.17 | 17.5 | 39.08 | 27.96 | 1.56 |
| Arg1 | 16.55 | 45.99 | 43.53 | 58.3 | 11.35 | 0.76 | 0.47 | 0.03 | 0.12 | 0.07 | 0.32 | 1.56 |
| Ppp1r1a | 8.74 | 34.74 | 28.59 | 27.07 | 30.32 | 27.72 | 87.95 | 0.08 | 1.01 | 3.44 | 2.25 | 1.57 |
| Mir466i | 6.45 | 0 | 42.13 | 0 | 0 | 0 | 0 | 0 | 0 | 0 | 0 | 1.57 |
| Snord69 | 876.29 | 817.97 | 2543.37 | 2657.96 | 0 | 1584.19 | 4424.62 | 2373.02 | 3354.96 | 2667.79 | 5805.25 | 1.57 |
| Cited1 | 9.17 | 68.46 | 31.23 | 27.41 | 111.27 | 319.97 | 271.32 | 69.84 | 80.49 | 350.26 | 161.27 | 1.58 |
| Syt7 | 3.91 | 18.4 | 13.95 | 13.38 | 16.09 | 5.69 | 3.34 | 1.34 | 1.85 | 0.24 | 0.76 | 1.58 |
| Ppm1e | 0.41 | 2.11 | 2.71 | 3.82 | 2.21 | 2.54 | 5.6 | 0.5 | 0.33 | 1.16 | 0.78 | 1.60 |
| Trim63 | 0.94 | 4.93 | 4.56 | 5.19 | 5.86 | 4.33 | 3.21 | 8.79 | 6.41 | 7.64 | 7.37 | 1.60 |
| Clrn1 | 0.03 | 3.22 | 2.26 | 2.03 | 4.46 | 3.15 | 1.08 | 0.12 | 0.06 | 0.13 | 0.14 | 1.61 |
| Meox1 | 30.78 | 111.58 | 95.92 | 97.19 | 67.82 | 38.64 | 32.63 | 0.49 | 1.22 | 0.5 | 0.27 | 1.62 |
| Mir6958 | 67.46 | 188.91 | 146.85 | 272.82 | 1147.05 | 0 | 0 | 219.22 | 0 | 0 | 0 | 1.62 |
| Slc16a6 | 5.94 | 14.14 | 19.07 | 21.76 | 12.26 | 5.34 | 6.44 | 1.39 | 1.49 | 5.44 | 7.3 | 1.63 |
| 9230105E05Rik | 0.36 | 2.85 | 2.84 | 3.59 | 2.92 | 2.4 | 2.1 | 2.2 | 0.92 | 2.81 | 1.6 | 1.63 |
| Fam19a5 | 3.71 | 15.34 | 14 | 13.27 | 16.25 | 15.56 | 16.99 | 4.75 | 4.81 | 6.59 | 7.11 | 1.64 |
| Nbl1 | 13.19 | 80.77 | 43.82 | 42.84 | 77.51 | 58.86 | 46.11 | 52.35 | 57.07 | 68.22 | 53.31 | 1.64 |
| Tnni1 | 11.47 | 75.62 | 38.9 | 37.36 | 60.85 | 31.09 | 14.22 | 40.96 | 36.56 | 22.01 | 22.2 | 1.65 |
| Snord49a | 195.65 | 3287.29 | 638.84 | 593.44 | 3881.2 | 1061.11 | 1077.69 | 953.68 | 1685.39 | 1786.91 | 1944.2 | 1.65 |
| Emp2 | 3.57 | 19.45 | 12.08 | 14.63 | 17.62 | 21.31 | 16.21 | 9.67 | 11.75 | 9.85 | 7.11 | 1.65 |
| Ripply2 | 0.74 | 5.15 | 4.1 | 5.02 | 1.56 | 1.74 | 20.5 | 13.14 | 13.46 | 31.33 | 16.89 | 1.68 |
| Fam213a | 35.34 | 126.07 | 104.01 | 126.61 | 130.26 | 126.87 | 147.91 | 27.22 | 30.28 | 34.36 | 33.91 | 1.68 |
| Atp10a | 1.18 | 7.7 | 5.7 | 6.27 | 7.08 | 4.47 | 2.27 | 4.26 | 6.22 | 5.01 | 5.38 | 1.68 |
| Zmat4 | 0.83 | 3.41 | 4.34 | 5.53 | 1.56 | 0.5 | 0.24 | 1.44 | 1.34 | 4.1 | 5.33 | 1.70 |
| Atp6v0e2 | 6.44 | 34.27 | 21.99 | 24.37 | 39.59 | 31.64 | 24.19 | 1.83 | 1.88 | 5.12 | 7.35 | 1.70 |
| Cldn9 | 7.54 | 46.14 | 26.29 | 27.5 | 39.67 | 44.2 | 18.91 | 0.93 | 2.6 | 43.63 | 36.7 | 1.71 |
| Lamp5 | 0.27 | 2.45 | 4.21 | 2.14 | 4.57 | 6.28 | 4.44 | 0.53 | 1.39 | 6.19 | 8.01 | 1.72 |
| Rpl5 | 7.94 | 18.76 | 23.99 | 33.26 | 22 | 17.24 | 50.94 | 63.76 | 39.11 | 30.6 | 33.24 | 1.73 |
| Ifitm1 | 8.01 | 53.99 | 27.85 | 30.46 | 57.55 | 83.88 | 83.72 | 120.69 | 92.17 | 65.77 | 56.65 | 1.74 |
| 1700108F19Rik | 3.84 | 19.53 | 12.62 | 17.9 | 17.3 | 11.19 | 10.57 | 0.13 | 0.24 | 0.38 | 0.2 | 1.75 |
| Doc2b | 2.07 | 12.48 | 9.87 | 8.77 | 13.01 | 5.79 | 2.55 | 1.1 | 1.73 | 2.06 | 1.25 | 1.75 |
| Lmx1b | 0.46 | 6.73 | 4.5 | 3.33 | 5.81 | 2.56 | 2.12 | 0.79 | 1.24 | 2.38 | 0.8 | 1.75 |
| Pdgfc | 14.48 | 39.23 | 38.43 | 64.02 | 42.28 | 34.78 | 75.92 | 32.4 | 39.3 | 32.08 | 42.48 | 1.75 |
| H2-Aa | 0.29 | 5.77 | 3.17 | 3.55 | 2.96 | 3.26 | 13.43 | 0.05 | 1 | 7.23 | 4.72 | 1.76 |
| Naaa | 3.86 | 20.29 | 14.19 | 16.71 | 18.33 | 19.35 | 23.93 | 32.39 | 58.31 | 55.39 | 72.07 | 1.76 |
| Fcgr3 | 0.57 | 2.2 | 6.28 | 2.46 | 1.42 | 20.31 | 75.4 | 0 | 0.07 | 0 | 0.12 | 1.77 |
| Lrrn1 | 2.55 | 11.15 | 9.36 | 13.34 | 14.28 | 9.75 | 18.24 | 0 | 0.2 | 2.11 | 0.96 | 1.80 |
| Sel1l3 | 9.46 | 49.17 | 34.63 | 36.57 | 53.42 | 34.54 | 19.18 | 4.64 | 7.38 | 34.07 | 34.23 | 1.81 |
| Bmp2 | 1.48 | 4.13 | 6.64 | 8.88 | 3.05 | 3.4 | 5.72 | 0.01 | 0 | 0.45 | 0.49 | 1.82 |
| Bcl2 | 6.87 | 20.79 | 21.36 | 32.72 | 20.88 | 12.68 | 21.15 | 12.91 | 13 | 15.78 | 15.2 | 1.83 |
| Snap91 | 7.42 | 14.47 | 23.4 | 34.71 | 13.32 | 11.52 | 79.93 | 46.08 | 33.29 | 42.15 | 34.95 | 1.84 |
| Tnnt2 | 0.18 | 5.8 | 3.6 | 2.84 | 10.29 | 4.82 | 1.26 | 0.44 | 0.35 | 0.42 | 1.2 | 1.84 |
| Rspo3 | 7.91 | 28.39 | 27.76 | 34.03 | 27.42 | 13.87 | 13.93 | 15.8 | 10.2 | 5.67 | 4.85 | 1.84 |
| Rxfp3 | 0.12 | 4.15 | 2.56 | 3.52 | 1.4 | 0.33 | 0.05 | 0.06 | 0 | 0.16 | 0.1 | 1.85 |
| Aif1 | 0.08 | 1.49 | 4.35 | 1.53 | 0.35 | 22.48 | 56.39 | 0 | 0 | 0 | 0 | 1.87 |
| Plcd3 | 1.69 | 13.25 | 9.53 | 8.22 | 11.73 | 9.59 | 7.25 | 8.95 | 9.19 | 14.22 | 11.8 | 1.88 |
| Rps6ka2 | 3.9 | 25.36 | 17.33 | 16.81 | 26.62 | 12.56 | 4.72 | 4.53 | 8.53 | 6.99 | 8.32 | 1.88 |
| Snord87 | 124.91 | 174.9 | 611.81 | 315.74 | 0 | 0 | 516.05 | 202.96 | 358.68 | 0 | 0 | 1.88 |
| Espn | 15.93 | 98.69 | 66.53 | 57 | 72.46 | 39.04 | 15.54 | 24.87 | 29.38 | 29.26 | 38.47 | 1.89 |
| Cdh4 | 1.66 | 10.39 | 9.42 | 8.34 | 10.58 | 8.62 | 8.07 | 6.11 | 5.94 | 12.73 | 13.51 | 1.89 |
| Sgms2 | 0.43 | 2.17 | 3.41 | 5.27 | 1.85 | 2.16 | 8.15 | 5.71 | 4.71 | 7.8 | 9.58 | 1.90 |
| Wnt4 | 9.74 | 27.04 | 46.96 | 31.74 | 38.82 | 57.48 | 56.27 | 3.05 | 7.59 | 27 | 49.74 | 1.91 |
| Mapk12 | 10.76 | 58.21 | 46.58 | 39.82 | 55.41 | 38.08 | 16.45 | 31.95 | 29.12 | 34.74 | 37.47 | 1.91 |
| Rubie | 1.94 | 6.14 | 10.24 | 9.88 | 7.05 | 3.65 | 3.99 | 4.6 | 5.7 | 0.49 | 4.2 | 1.91 |
| H2-Eb1 | 0.62 | 10.79 | 5.8 | 4.46 | 5.78 | 5.28 | 10.9 | 0.43 | 2.58 | 14.41 | 11.71 | 1.92 |
| Dcx | 0.75 | 5.39 | 5.28 | 6.02 | 7.25 | 3.5 | 4.56 | 0.3 | 0.14 | 0.06 | 0.06 | 1.93 |
| C2cd4b | 0.71 | 6.81 | 5.35 | 5.93 | 6.48 | 2.71 | 8.88 | 16.88 | 16.77 | 19.11 | 16.93 | 1.96 |
| Sypl2 | 0.14 | 5.95 | 3.44 | 3.48 | 5.49 | 2.37 | 0.86 | 2.44 | 2.43 | 5.35 | 5.1 | 1.97 |
| Ppp1r16b | 0.99 | 6.3 | 5.94 | 7.72 | 7.29 | 6.38 | 5.82 | 0.53 | 0.7 | 1.29 | 2.32 | 1.98 |
| Cited2 | 37.18 | 78.26 | 111.54 | 188.89 | 56.85 | 37.34 | 53.66 | 88.27 | 60.32 | 64.42 | 71.59 | 1.99 |
| Aard | 2.2 | 9.52 | 9.44 | 13.95 | 10.93 | 4.94 | 6.04 | 0.23 | 0.14 | 0.73 | 1.19 | 1.99 |
| Slc45a3 | 2.2 | 17.64 | 12.08 | 11.45 | 17.06 | 15.12 | 13.4 | 6.44 | 5.57 | 22.66 | 19.96 | 2.00 |
| Dio3 | 0.33 | 2.49 | 3.17 | 5.46 | 1.63 | 1.25 | 0.18 | 0.64 | 0.9 | 1.8 | 1.55 | 2.00 |
| Sall1 | 19.94 | 62.57 | 80.66 | 85.52 | 64.01 | 46.78 | 50.45 | 40.77 | 45.36 | 82.21 | 80.72 | 2.01 |
| Pax8 | 12.09 | 47.57 | 58.59 | 44.76 | 51.59 | 59.89 | 71.31 | 26.26 | 32.41 | 62.09 | 52.33 | 2.01 |
| Gpsm3 | 5.2 | 43.25 | 27.57 | 20.5 | 38.18 | 28.44 | 21.7 | 2.58 | 6.01 | 8.13 | 8.61 | 2.01 |
| Hspa1a | 19.08 | 18.58 | 57.69 | 102.64 | 26.95 | 49.25 | 109.52 | 1.77 | 0.55 | 0.84 | 0.84 | 2.02 |
| Fcer1g | 3.44 | 7.94 | 24.76 | 9.15 | 4.62 | 97.84 | 243.93 | 1.08 | 1.39 | 1.65 | 1.2 | 2.02 |
| Sobp | 1.35 | 7.89 | 8.03 | 9 | 8.73 | 4.79 | 2.97 | 7.43 | 7.35 | 9.8 | 8.18 | 2.02 |
| Ppbp | 0.47 | 0 | 5.75 | 4.17 | 0.09 | 1.02 | 1.77 | 0.66 | 0.54 | 0.48 | 1.09 | 2.02 |
| Gm5082 | 0.02 | 4.01 | 2.83 | 3.45 | 1.46 | 0.3 | 0.95 | 1.19 | 1.22 | 0 | 0.22 | 2.02 |
| Fcrls | 0.26 | 2.23 | 6.41 | 1.98 | 1.3 | 15.01 | 26.52 | 0 | 0 | 0 | 0 | 2.04 |
| Bbx | 4.76 | 15.56 | 16.49 | 29.36 | 13.36 | 8.05 | 11.29 | 17.49 | 14.54 | 10.32 | 10.03 | 2.05 |
| Pf4 | 2.42 | 2.4 | 14.44 | 12.4 | 2.63 | 55.31 | 153.12 | 1.27 | 1.23 | 0.22 | 0 | 2.08 |
| Eno3 | 24.65 | 181.86 | 107.37 | 107.85 | 132.11 | 48.5 | 21.36 | 31.81 | 43.04 | 12.48 | 32.36 | 2.08 |
| A730017C20Rik | 5.95 | 23.08 | 28.76 | 28.43 | 28.09 | 26.34 | 33.06 | 1.66 | 6.52 | 13.77 | 13.14 | 2.09 |
| Ism1 | 3.36 | 18.48 | 15.17 | 20.08 | 19.38 | 18.71 | 13.75 | 0.34 | 0.97 | 2.51 | 4.49 | 2.09 |
| Shroom3 | 6.93 | 39.96 | 35.04 | 30.74 | 35.22 | 20.23 | 19.44 | 7.29 | 11.39 | 16.84 | 16.83 | 2.10 |
| Pcdh8 | 2.34 | 14.52 | 10.46 | 16.18 | 11.01 | 6.16 | 2.59 | 0.72 | 1.73 | 3.97 | 5.43 | 2.10 |
| Vat1l | 2.55 | 16.41 | 13.41 | 15.08 | 13.5 | 8.31 | 3.9 | 38.08 | 29.76 | 41.22 | 34.8 | 2.10 |
| Cftr | 0.21 | 3.45 | 3.53 | 4.91 | 4.16 | 4.3 | 5.78 | 3.42 | 3.06 | 3.87 | 2.34 | 2.11 |
| Oxnad1 | 13.29 | 66.95 | 55.11 | 66.55 | 58.31 | 40.51 | 29.14 | 22.86 | 20.96 | 18.86 | 18.76 | 2.11 |
| Cdo1 | 11.8 | 62.79 | 49.05 | 60.36 | 51.36 | 37.83 | 41.26 | 14.2 | 20.35 | 4.34 | 9.24 | 2.12 |
| Apom | 1.13 | 19.06 | 8.87 | 7.75 | 21.57 | 21.8 | 20 | 0.7 | 3.54 | 3.92 | 1.86 | 2.13 |
| Capn6 | 43.3 | 283.73 | 190.38 | 205.86 | 295.79 | 212.72 | 198.42 | 66.35 | 62.36 | 161.28 | 138.32 | 2.17 |
| 2610028E06Rik | 0.11 | 4.96 | 3.95 | 4.03 | 5.28 | 6.74 | 5.72 | 1.61 | 1.86 | 7.25 | 6.69 | 2.17 |
| Snora23 | 0 | 0 | 5.68 | 1.32 | 0 | 0 | 0 | 2.12 | 0 | 0 | 0 | 2.17 |
| Pdlim3 | 0.42 | 8.82 | 4.45 | 6.35 | 5.99 | 9.36 | 8.43 | 0.23 | 0.52 | 1.15 | 2.18 | 2.17 |
| C2cd4a | 0.17 | 5.08 | 2.86 | 5.74 | 4.19 | 3.95 | 4.9 | 17.1 | 16.93 | 36.04 | 25.26 | 2.18 |
| E030013I19Rik | 0.35 | 2.54 | 4.63 | 5.65 | 4.75 | 6.02 | 8.62 | 0.61 | 0.64 | 3.57 | 3.13 | 2.19 |
| Foxd2os | 4.82 | 33.66 | 25.59 | 25.36 | 31.04 | 20.08 | 14.89 | 0.13 | 1.59 | 0.87 | 1.48 | 2.19 |
| Gm266 | 28.1 | 132.66 | 133.92 | 132.59 | 125.04 | 79.74 | 89.68 | 44.21 | 42.65 | 85.31 | 81.33 | 2.21 |
| Shisa2 | 4.55 | 21.6 | 21 | 28.5 | 23.41 | 19.93 | 39.55 | 7.11 | 4.6 | 9.24 | 10.37 | 2.21 |
| Lbx2 | 1.7 | 13.52 | 12.5 | 10.66 | 12.16 | 8.12 | 1.97 | 8.5 | 8.21 | 5.99 | 12.43 | 2.22 |
| Rnase12 | 1.28 | 10.57 | 10.14 | 9.18 | 17.07 | 20.91 | 62.7 | 1.17 | 3.46 | 34.96 | 33.52 | 2.23 |
| Des | 27.53 | 232.18 | 157 | 115.38 | 206.98 | 79.77 | 19.92 | 370.77 | 516.15 | 671.9 | 573.06 | 2.27 |
| H2-Ab1 | 1.39 | 21.1 | 11.85 | 9.33 | 13.5 | 15.48 | 23.31 | 21.07 | 78.52 | 134.78 | 87.41 | 2.28 |
| Tmem37 | 4.92 | 43.69 | 26.52 | 28.96 | 20.34 | 17.36 | 28.49 | 12.43 | 12.19 | 41.55 | 40.52 | 2.28 |
| Ntrk3 | 2.35 | 22.55 | 16.49 | 14.05 | 27.02 | 16.48 | 9.83 | 0.05 | 0.35 | 2.48 | 1.6 | 2.28 |
| Hspa1b | 14.14 | 15.61 | 44.91 | 100.36 | 21.16 | 37.24 | 130.47 | 2.32 | 0.85 | 0.99 | 0.78 | 2.28 |
| Kif26b | 2.67 | 24.08 | 16.34 | 17.83 | 22.74 | 16.64 | 17.3 | 11.13 | 10.4 | 18.13 | 16.51 | 2.30 |
| Amigo1 | 1.36 | 14.5 | 10.86 | 11.18 | 13.33 | 8.05 | 5.11 | 7.26 | 8.54 | 10.03 | 7.95 | 2.35 |
| Elavl4 | 1.12 | 3.51 | 7.38 | 12.47 | 3.75 | 4.16 | 14.82 | 8.96 | 2.03 | 6.09 | 3.93 | 2.37 |
| Snord57 | 173.64 | 0 | 944.97 | 877.82 | 1968.38 | 0 | 0 | 846.41 | 997.21 | 0 | 1725.53 | 2.39 |
| C130021I20Rik | 0.31 | 9.86 | 6.56 | 5.21 | 7.79 | 4.96 | 3.48 | 0.36 | 0.85 | 3.33 | 2.19 | 2.39 |
| Robo2 | 7.64 | 28.97 | 35.56 | 53.38 | 30.47 | 17.07 | 22.76 | 50.18 | 33.06 | 19.44 | 15.33 | 2.40 |
| Rem1 | 8.56 | 71.8 | 52.08 | 46.91 | 85.37 | 74.41 | 29.16 | 52.83 | 44.66 | 117.77 | 79.51 | 2.40 |
| Actn3 | 2.8 | 28.49 | 22.06 | 16.83 | 19.63 | 6.81 | 1.45 | 9.29 | 16.39 | 8.27 | 11.01 | 2.43 |
| Btbd11 | 5 | 41.69 | 27.74 | 34.84 | 38.57 | 22.37 | 22.12 | 35.86 | 37.31 | 72.38 | 67.7 | 2.43 |
| Tpm2 | 37.37 | 293.37 | 210.21 | 204.47 | 286.54 | 199.68 | 86.52 | 96.33 | 97.99 | 125.85 | 106.37 | 2.44 |
| Dock5 | 1.27 | 14.42 | 10.14 | 12.87 | 18.31 | 13.36 | 5.16 | 4.58 | 4.55 | 2.92 | 2.84 | 2.46 |
| Moxd1 | 5.46 | 42.06 | 35.82 | 34.78 | 53.95 | 14.97 | 2.65 | 3.17 | 5.69 | 2.41 | 7.43 | 2.49 |
| Rbfox1 | 0.75 | 7.49 | 8.73 | 9.39 | 7.58 | 4.19 | 6.95 | 6.86 | 4.68 | 8.86 | 5.54 | 2.52 |
| Foxd2 | 8.02 | 58.31 | 50.71 | 52.4 | 44.6 | 28 | 18.35 | 0.26 | 1.99 | 1.14 | 1.01 | 2.54 |
| Ngfr | 9.13 | 80.75 | 57 | 60.55 | 58.91 | 23.29 | 14.78 | 55.92 | 71.63 | 82.7 | 76.75 | 2.56 |
| Fxyd6 | 61.67 | 610.02 | 370.18 | 385.52 | 551.52 | 349.64 | 276.41 | 122.95 | 179.37 | 135.59 | 175.5 | 2.60 |
| Rasef | 0.75 | 6.4 | 7.23 | 12 | 5.97 | 4.1 | 4.1 | 2.52 | 1.17 | 1.01 | 1.18 | 2.60 |
| Hs3st6 | 2.59 | 34.51 | 22.87 | 19.11 | 40.81 | 41.9 | 19.91 | 44.43 | 42.13 | 25.62 | 28.94 | 2.61 |
| Fgf1 | 0.44 | 11.06 | 7.71 | 8.52 | 15.78 | 29.1 | 47.53 | 7.7 | 5.16 | 25.36 | 9.92 | 2.66 |
| Car4 | 1.52 | 16.94 | 17.06 | 12.92 | 11.12 | 14.51 | 24.34 | 27.38 | 24.84 | 18.19 | 37.89 | 2.67 |
| Tpbg | 11.19 | 58.1 | 63.96 | 93.63 | 47.43 | 18.98 | 20.61 | 40.19 | 54 | 53.07 | 49.72 | 2.71 |
| Itga8 | 3.09 | 20.9 | 20.92 | 30.83 | 25.49 | 23.1 | 51.16 | 8.89 | 6.72 | 29.56 | 24.88 | 2.72 |
| C1qa | 1.03 | 7.25 | 21.06 | 3.69 | 3.1 | 96.72 | 182.33 | 0 | 0 | 0.57 | 0 | 2.72 |
| 1110032F04Rik | 1.51 | 11.28 | 13.76 | 18.36 | 11.42 | 5.98 | 5.99 | 3.15 | 2.78 | 2.18 | 2.75 | 2.76 |
| Fgf10 | 1.46 | 7.27 | 10.81 | 21.41 | 6.9 | 2.96 | 7.34 | 2.45 | 1.42 | 0.84 | 1.4 | 2.80 |
| Snora34 | 14.29 | 122.39 | 139.97 | 73.03 | 62.14 | 58.12 | 108.22 | 24.8 | 69.84 | 148.06 | 108.97 | 2.81 |
| Ajap1 | 0.99 | 20.91 | 12.79 | 13.24 | 21.35 | 17.68 | 9.73 | 2.61 | 2.67 | 1.81 | 0.9 | 2.82 |
| Amph | 1.74 | 19.71 | 17.83 | 19.08 | 17.78 | 19.94 | 22.49 | 7.81 | 10.76 | 25.37 | 24.23 | 2.83 |
| Colq | 0.12 | 13.01 | 7.03 | 7.05 | 11.78 | 10.14 | 4.27 | 1.35 | 1.73 | 1.81 | 2.79 | 2.84 |
| Has2 | 5.09 | 34.63 | 36.03 | 49.85 | 36.42 | 17.42 | 17.36 | 7.34 | 12.68 | 19.61 | 23.89 | 2.85 |
| Defb36 | 0 | 10.45 | 4.74 | 8.07 | 10.87 | 19.97 | 7 | 4.04 | 4.17 | 21.79 | 12.37 | 2.89 |
| Cpa1 | 1.24 | 33.62 | 14.74 | 16.49 | 15.03 | 4.06 | 0.47 | 17.14 | 49.42 | 3.46 | 43.2 | 2.89 |
| Tekt5 | 0.12 | 14.42 | 7.58 | 7.32 | 9 | 4.26 | 1.87 | 0.43 | 0.5 | 0 | 0.26 | 2.92 |
| 2310015B20Rik | 1.72 | 36.89 | 14.71 | 24.55 | 40.58 | 37.58 | 22.71 | 0.56 | 0 | 3.49 | 3.41 | 2.92 |
| Pla2g7 | 12.01 | 127.35 | 85.15 | 110.88 | 95.26 | 69.82 | 125.95 | 27.84 | 53.39 | 108.28 | 100.42 | 2.93 |
| Eya2 | 1.95 | 38.27 | 24.26 | 20.89 | 37.31 | 22.23 | 20.44 | 0.54 | 3.03 | 3.33 | 6.42 | 3.00 |
| Cutal | 0.36 | 9.68 | 8.8 | 11.47 | 9.39 | 10.43 | 15.22 | 30.2 | 22.77 | 95.1 | 57.42 | 3.03 |
| C1qb | 1.92 | 13.7 | 34.87 | 11.31 | 5.16 | 160.04 | 312.62 | 0.1 | 0.18 | 0.19 | 0.64 | 3.04 |
| C1qc | 1.35 | 10.74 | 30.07 | 8.96 | 5.12 | 130.33 | 252.61 | 0 | 0 | 0 | 0.32 | 3.13 |
| Traf1 | 0.87 | 24.96 | 16.4 | 14.4 | 31.71 | 45.86 | 33.84 | 0.23 | 1.78 | 27.61 | 21.31 | 3.13 |
| 4930500L23Rik | 0.2 | 6.66 | 10.2 | 9.32 | 6.74 | 6.87 | 8.65 | 4.03 | 1.89 | 1.23 | 0.25 | 3.16 |
| Defb19 | 0.14 | 15.72 | 6.42 | 12.08 | 2.45 | 2.73 | 19.43 | 5.85 | 10.75 | 3.94 | 27.18 | 3.17 |
| Fam189a2 | 1.16 | 29.73 | 16.1 | 20.84 | 29.22 | 22.07 | 12.94 | 0.36 | 2.25 | 4.56 | 5.46 | 3.17 |
| Pisd-ps3 | 0.25 | 0.07 | 17.4 | 3.72 | 7.12 | 33.35 | 0.1 | 2.1 | 0.03 | 14.38 | 47.62 | 3.21 |
| Pnmt | 0.2 | 21.98 | 9.77 | 10.61 | 12.26 | 15.31 | 26.97 | 0.07 | 0.12 | 0.73 | 0.2 | 3.22 |
| Cdhr1 | 0.31 | 20.46 | 11.79 | 11.75 | 16.23 | 10.93 | 6.74 | 18.46 | 15.01 | 12.39 | 7.54 | 3.29 |
| Hoxb1 | 0.5 | 9.34 | 12.45 | 16.88 | 1.82 | 0.24 | 0.24 | 0.02 | 0 | 0 | 0 | 3.38 |
| Fgf20 | 0.6 | 6.51 | 10.13 | 21.52 | 5.25 | 3.33 | 6.41 | 0 | 0.06 | 1.84 | 1.93 | 3.39 |
| Nell1 | 2.1 | 51.67 | 27.62 | 35.68 | 44.97 | 30 | 21.55 | 19.91 | 26.67 | 20.7 | 20.31 | 3.40 |
| Osr1 | 7.04 | 114.7 | 92.6 | 81.57 | 109.25 | 71.35 | 14.62 | 39.2 | 55.26 | 38.5 | 34.13 | 3.45 |
| Phf19 | 2.6 | 48.25 | 39.74 | 40.31 | 58.2 | 29.04 | 11.1 | 13.14 | 13.12 | 13.56 | 21.05 | 3.51 |
| Gpx6 | 1.29 | 25.82 | 21.96 | 29.48 | 7.19 | 10.96 | 99.68 | 44.18 | 125.01 | 370.01 | 513.93 | 3.54 |
| Esr1 | 0.22 | 20.31 | 12.65 | 14.29 | 19.35 | 13.51 | 10.19 | 18 | 15.85 | 22.24 | 18.89 | 3.57 |
| Spock2 | 3.76 | 115.89 | 70.1 | 63.55 | 116 | 85.57 | 58.51 | 8.18 | 11.2 | 15.2 | 11.63 | 3.83 |
| Meox2 | 2.59 | 26.16 | 37.38 | 63.14 | 25.46 | 11.58 | 8.55 | 65.58 | 27.01 | 4.16 | 6.35 | 3.84 |
| Pcp4 | 1.13 | 34.23 | 26.4 | 35.23 | 34.64 | 88.38 | 120.83 | 0.41 | 8.46 | 502.99 | 353.15 | 3.90 |
| Snord8 | 0 | 42.02 | 16.33 | 15.17 | 0 | 81.39 | 0 | 24.38 | 0 | 0 | 0 | 4.07 |
| Defb25 | 2.33 | 152.03 | 54.01 | 57.84 | 123.25 | 208.16 | 194.94 | 37.94 | 42.74 | 670.88 | 436.88 | 4.10 |
| Mir1668 | 0 | 90.14 | 11.68 | 21.7 | 0 | 0 | 0 | 17.43 | 92.43 | 32.67 | 0 | 4.14 |
| Rspo1 | 5.25 | 170.42 | 117.47 | 107.64 | 165.32 | 121.67 | 91.05 | 5.95 | 13.52 | 19.09 | 17.88 | 4.18 |
| Fgf8 | 0.92 | 53.19 | 35.91 | 32.87 | 52.58 | 34.86 | 30.65 | 2.27 | 5.19 | 5.61 | 17.63 | 4.20 |
| AU015791 | 0.46 | 39.76 | 33.94 | 21.39 | 38.72 | 18.38 | 33.31 | 17.1 | 8.71 | 17.38 | 18.43 | 4.30 |
| Eya1 | 4.61 | 75.2 | 89.86 | 134.7 | 73.27 | 47.66 | 63.65 | 44.08 | 41.9 | 32.66 | 30.53 | 4.34 |
| Uncx | 7.53 | 201.61 | 203.98 | 145.38 | 183.68 | 117.66 | 78.51 | 32.41 | 36.24 | 35.95 | 28.25 | 4.36 |
| 1600027J07Rik | 2.21 | 69.95 | 49.04 | 86.35 | 62.89 | 26.57 | 22.62 | 0.31 | 2.21 | 2.34 | 0.95 | 4.42 |
| Mir1895 | 0 | 0.05 | 29.86 | 11.39 | 0 | 131.55 | 0 | 21 | 130.93 | 112.36 | 0 | 4.43 |
| Six2 | 7.35 | 244.12 | 235.96 | 225.05 | 252.43 | 159.63 | 116.68 | 302.27 | 233.21 | 284.37 | 250.71 | 4.79 |
| Crym | 2.24 | 215.89 | 101.23 | 123.48 | 259.52 | 234.56 | 75.46 | 203.17 | 155.86 | 407.09 | 266.58 | 5.13 |
| Snord7 | 0 | 12.09 | 13.83 | 66.37 | 32.3 | 29.11 | 42.44 | 0 | 106.07 | 42 | 0 | 5.36 |
| AF357399 | 0 | 0 | 61.76 | 19.12 | 0 | 0 | 0 | 61.47 | 0 | 0 | 0 | 5.37 |
| Mir6990 | 0 | 0 | 71.66 | 44.38 | 248.77 | 0 | 0 | 142.63 | 126.03 | 133.62 | 218.08 | 5.88 |
| Mir1291 | 0 | 59.71 | 32.21 | 88.93 | 103.37 | 0 | 61.12 | 22.4 | 12.66 | 4.34 | 70.94 | 5.94 |
| Mir1949 | 0 | 0 | 93.97 | 87.29 | 0 | 0 | 594.45 | 0 | 0 | 0 | 0 | 6.52 |
| Snord88a | 0 | 409.09 | 79.5 | 147.7 | 0 | 0 | 0 | 0 | 0 | 0 | 0 | 6.84 |
| Snord95 | 0 | 0 | 337.36 | 208.93 | 292.8 | 840.53 | 0 | 0 | 0 | 0 | 0 | 8.10 |
| Snord53 | 0 | 0 | 337.36 | 313.39 | 0 | 0 | 0 | 167.87 | 0 | 0 | 0 | 8.35 |

TABLE 2
Primer Sequences
qRT-PCR primers

| Gene | | |
|---|---|---|
| Name | Forward Primer | Reverse Primer |
| Gapdh | CATGGCCTTCCGTGTTCCTA | CCTGCTTCACCACCTTCTTG |
| | (SEQ ID NO: 2) | AT (SEQ ID NO: 3) |
| Lhx1 | CTTCTTCCGATGTTTCGGTA | TCATGCAGGTGAAGCAGTTG |
| | (SEQ ID NO: 4) | (SEQ ID NO: 5) |
| Pax8 | GGCTCTACCTACTCTATCAA | CTGCTGCTGCTCTGTGAGTC |
| | (SEQ ID NO: 6) | (SEQ ID NO: 7) |
| Six2 | AGGAAAGGGAGAACAGCGAG | GGACTGGACGACGAGTGGT |
| | AA (SEQ ID NO: 8) | (SEQ ID NO: 9) |
| Wt1 | CCACACCCCTACTGACAGTT | TCACTCTCATACCCTGTGCC |
| | (SEQ ID NO: 10) | (SEQ ID NO: 11) |
| Osr1 | CTGCCCAACCTGTATGGTTT | TGGCACTTTAGAAAAAGAGG |
| | (SEQ ID NO: 12) | (SEQ ID NO: 13) |
| Cd2ap | ACGCAGTGCTTTCCAAACC | CCCGCAAGTGGATGTCTGG |
| | (SEQ ID NO: 14) | (SEQ ID NO: 15) |

TABLE 3
Reagents

| Antibodies | Company | Cat. No. | Dilutions |
|---|---|---|---|
| SIX2 | Proteintech | 11562-1-AP | (1:200) |
| SALL1 | R&D | PP-K9814-00 | (1:200) |
| CITED1 | Life Technologies | PA1-24469 | (1:200) |
| WT1 | Abcam | ab89901 | (1:200) |
| PAX2 | Covance | PRB-276P | (1:200) |
| LHX1 | Developmental Studies | 4F2 | (1:100) |
| | Hybridoma Bank | | |
| PAX8 | Proteintech | 10336-1-AP | (1:100) |
| PODXL | R&D | MAB1556 | (1:100) |
| (mouse) | | | |
| PODXL | R&D | AF1658 | (1:100) |
| (human) | | | |
| LTL | Vector laboratories | B-1325 | (1:200) |
| DBA | Vector laboratories | B-1035 | (1:200) |
| CDH1 | Cell Signling | 3195 | (1:200) |
| SYNPO | PROGEN Biotechnik | 65294 | (1:100) |
| NPHS1 | PROGEN Biotechnik | GP-N2 | (1:100) |
| AQP1 | Santa Cruz | sc-25287 | (1:200) |
| CD31 | BD Biosciences | 553370 | (1:100) |
| HuNu | Millipore | MAB1281 | (1:100) |
| EpCAM | R&D | FAB9601G | follow product |
| | | | instructions |
| NGFR | BioLegend | 345106 | follow product |
| | | | instructions |
| CK8 | Covance | MMS-162P-250 | (1:200) |

| Reagents | Company | Cat. No. |
|---|---|---|
| BMP7 | R&D | 354-BP-010 |
| FGF2 | R&D | 233-FB-025 |
| Heparin | Sigma | H3149-100KU |
| Y27632 | TOCRIS | 1254 |
| mLIF | Millipore | ESG1107 |
| hLIF | Millipore | LIF1050 |
| CHIR99021 | REAGENTS DIRECT | 27-H76 |

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

1. A method of producing a self-renewable nephron progenitor cell (srNPC) comprising: a) purifying a population of Six2+ cells from a mammal; b) culturing the population of Six2+ cells in a composition comprising nephron progenitor self-renewal (NPSR) media; and c) isolating the floating aggregated cells.

2. The method of claim 1, wherein the cells are dissociated and passaged every 1 to 14 days.

3. (canceled)

4. The method of claim 1, wherein the population of Six2+ cells is cultured at a density of 100 to 20000 cells per well in a 96-well plate, or equivalent density in a smaller or larger plate.

5. (canceled)

6. The method of claim 4, wherein the 96-well plate is a U bottom low-attachment plate.

7.-13. (canceled)

14. A method of producing a self-renewable nephron progenitor cell (srNPC) comprising: a) purifying a population of EpCAM−/NGFR+ cells; b) culturing the population of EpCAM−/NGFR+ cells in a composition comprising nephron progenitor self-renewal (NPSR) media; and c) isolating the floating aggregated cells.

15. The method of claim 14, wherein the cells are passaged every 1 to 14 days.

16. (canceled)

17. The method of claim 14, wherein the population of EpCAM−/NGFR+ cells are cultured at a density of 100 to 50000 cells per well of a 96-well plate, or an equivalent density in a smaller or larger plate.

18. (canceled)

19. The method of claim 17, wherein the 96-well plate is a U bottom low attachment plate.

20. The method of claim 1, wherein the NPSR media comprises a BMP, a FGF, a heparan sulfate, a kinase inhibitor, a cytokine, or combinations thereof.

21. The method of claim 1, wherein the NPSR media comprises BMP7, FGF2, Heparin, Y27632, LIF, CHIR99021, or combinations thereof.

22. The method of claim 1, wherein the population of cells is derived from an embryonic kidney, a fetal kidney, an adult kidney, or menstrual fluid.

23. (canceled)

24. The method of claim 1, wherein the cells are derived from a mouse, rat, pig, dog, cat, rabbit, cow, horse, camel, goat, or human.

25. The method of claim 14, wherein the population of cells is Six2+.

26. A method of producing a fate-specified nephron progenitor cell comprising contacting a self-renewable nephron progenitor cell (srNPC) made by a method of claim 1 with a spinal cord tissue, a kinase inhibitor, a growth factor, or a combination thereof.

27. The method of claim 26, wherein the srNPC is contacted with a spinal cord tissue, CHIR99021, FGF2, or combinations thereof.

28. (canceled)

29. A method of treating a subject with a kidney disease comprising administering an effective amount of the srNPC or FS-NPC made by the method of claim 1.

30. The method of claim 29, wherein the kidney disease is acute or chronic.

31. The method of any of claim 29, wherein the kidney disease is one or more disease selected from the group consisting of analgesic nephropathy, diabetic nephropathy, IgA nephropathy, contrast induced nephropathy, lithium induced nephropathy, xanthine oxidase deficiency, polycystic kidney disease, onconephrology, renal insufficiency, renal failure, and uremia.

32.-35. (canceled)

36. A method of screening a drug for kidney toxicity comprising a) contacting a nephron organoid with the drug and b) detecting signs of toxicity, wherein the nephron organoid is created by contacting a srNPC made by the method of claim 1 with a composition comprising CHIR99021 and FGF2.

37. (canceled)

38. The method of claim 36, wherein the signs of toxicity comprise changes in glomerulus, proximal tubule, distal tubule, loop of Henle morphology, or cellular apoptosis.

39-51. (canceled)
